SEQUENCE LISTING (65 sequences)

SEQ ID NO: 1 - protein, 128 aa, Nicotiana tabacum
MGLKGKLISQMEMKCAGDLLHEHFKSNPHQTSTMSPDKITNFTLHEGQLGNTGSVVSWKYVLGGKERHAKQALHIDDAKKSITFNFLEGYMNELYKSMTPQYRINNNLECHKSRNHPMQVTSPNHTQI

SEQ ID NO: 2 - protein, 540 aa, Nicotiana tabacum
MKAEGSALSSAGSYHRLAYHEVINDDNQNKIFTSDDSRLRQLGYKQELYRGLSFIANFSFTFAIVSVLTGISTLYNQALTFGGPITLVYGWPIVSLMTLIVGLAMAEICSAYPTSAGLYYWSAKLSGNYFGPFASWITGWFNIVGQWAVTASIDFSLAQLVQVMILLSTGGLNGGGYQASKYVVIALHGGILLLHAILNSLPISWLSFFGQLAAAWNVLGVFLLMILIPMVSTERASAKFVFTNFNTDNGDGINNNLYIFVLGLLMSQYTLTGYDASAHMTEETKNADKNGPKGIVSAIGISVLAGWAYILGITFAVTDIPHLLNKNNDSGGYAIAQIFYDAFKNRYGSGVGGIICLGVIAIAVFFCGMSSLTSNSRMAYAFSRDGAMPYSSFWHKVNKQEVPLNAVWMSAFIAFCMALTSLGSLVAFQAMTSIATIGLYIAYALPILFRVTLARKSFTPGPFNLGSYGLVVGWVAIFWVALISVLFSLPVAYPITDQTLNYTPVAVGGLLILVVSSWIFSAIHWFKGPITNLGNSSEEA

SEQ ID NO: 3 - protein, 145 aa, Nicotiana tabacum
MASTQQAVSSGSDADQRYAKFDERKRKRMESNRESARRSRMRKQQRLGELMSETTQLQNQNSICRERIDSVERNYCAIDAENNVLRAQIAELTERLNSLNSLTQFWADATGFPVDLPEIPDTLLEPWQLPCPIQPIDASSDMLLF

SEQ ID NO: 4 - protein, 654 aa, Nicotiana tabacum
MPGVYLETASLPKGRGLECQESQAVRYFFRGRNKVDDSLTIEIFNLFPWIFFTILAMDKHHHQLPLTKSTSRQRYNEWVFRDVPSDITIEVDGGIFSLHKFPLVSRSGRIRRLVAEHRDSDISRIELVSLPGGTESFELAAKFCYGVNFEITAANVAQLCCVSDYLEMSEDYSKNNLGSRAEEYLDSIVCKNLEMCVEVLRQCENLLPLADELKVVSRCIDAVASKACVEQIASSFSRLEYSISGGRLHMSKQANCELDWWIEDISMLRIDLYQRVITAMKFRGVRPESIAASLVNYAQKELIQKTLSGSNIQEKLVVETIVSLMPVEKFVVPLTFLFGLLRSAVMLDCTVACRLDLERRIGSQLDTATLDDILIPSFRHAGDTLFDVDTVHRILVNFSQQEGDSDDDMEDVSVFESDSPTTTPSQTALFKVSKLVDNYLAEIALDANLKLNKFIAVAETLPAHARTVHDGLYRAIDLYLKAHQTLSDPDKRRLCKLIDFQKLSQEAGAQAAQNERLPLQSIVQVLYFEQLRLRNALFCSYPDDDIKPTHQSWRINSGALSAAMSPKDNYASLRRENRELKLELARMRMRLNDLEKDHVCMKRNMQKSSSRRFMKSFSKRIGKKFNIFGHNFSRDCSSPSSQSERTESKITERT

SEQ ID NO: 5 - protein, 303 aa, Nicotiana tabacum
MEHSAADRDPKAVEFAKDKNGVGQVLLRNPRGASVRVSLHGGQVLSWKNDHGEELLFISSKATFKPPTAVRGGIPICFPQFGNRGSLEQHGFARNRMWIIDDNPPPLHPNDSNGKAFTDLLLKSSDDDLKVWPHGFEFRLRVTLAVDGSLTLISRIRNVNCKPFSFSIAYHTYFALSDISEVRVEGLETLDYLDNLCNRERFTEQGDALTFETEVDRVYLSSSDVIAIFDHEKKRTFVIKREGLPDVVVWNPWEKKSKTIADFGDDEYRHMLCVDGAAIEKPITLKPGEEWTGRLELSVMPSS

SEQ ID NO: 6 - protein, 578 aa, Nicotiana tabacum
MEVMKKKENTSRKMKGGMITMPFIFANEICEKLAVVGFGANMIIYLTNELHLPLTKAANTLTNFGGTASLTPLLGAFIADTFAGRFWTITIASIIYQIGMIILTVSAILPQLRPPSCKGDEFCKEANSGQLAILYISLLLTAFGSGGIRPCVVAFGAEQFDETDPNQKTQTWKFFNWYYFSMGFSMLIAVTVIVYIQDNIGWGIGFGVPTIAMLISIIVFIFGYPLYRNLDPAGSPFTRLLQVCVAAYKKRKLDMVSDPSFLYQNEELDSAISTAGKLVHTKQMKFLDRAAIVTEEDNRKSPNLWRLNTVHRVEELKSIIRMGPIWASGIILITAYAQQHTFSVQQAKTMDRHLINSFEIPAASMTVFTLTAMLCTICFYDRVFVPIARKFTGLERGISFLSRMAIGFSISVLATLVAGFIEVKRKEAALTHGLIDKGKAIVPISVFWLVPQYCLHGVVAFMSIGHLEFFYDQAPESMRSTATALFWTSISAGNYLSTLLVSLVHKFTSGSGGSNWLPDNNLNKGKLEYFYWLITILQVVNLIYYLFCAKFYTFKPIQVHKTEDLDSKKDSIELVNNV

SEQ ID NO: 7 - protein, 517 aa, Nicotiana tabacum
MALSNTLSLSSSKSLVQSHLLHNPSLPQPRIPVFHNPQHGRRHPISAVHAAEPAKTATASQPLKKTQWSLDSWKSKKALQLPEYPDEKELESVLETLESNPPLVFAGEARNLEERLGEAALGKAFLLQGGDCAESFKEFNANNIRDTFRILLQMSVVLMFGGQVPVIKVGRMAGQFAKPRSDPFEEIDGVKLPSYKGDNINGDTFDEKSRIPDPHRLIRAYMQSAATLNLLRAFATGGYAAMQRVTEWNLDFVENSEQGDRYQELAHRVDEALGFMAAAGLTVDHPIMATTDFWTSHECLLLPYEQALTREDSTSGLFYDCSAHMIWVGERTRQLDGAHVEFLRGVANPLGIKVSQKMDPNELVKLIDILNPTNKPGRITVIVRMGAENMRVKLCHLIRAVRGAGQIVTWVCDPMHGNTIKAPCGLKTRAFDSILAEVRAFFDVHEQEGSHPGGIHLEMTGQNVTECIGGSRTVTYDDLGSRYHTHCDPRLNASQSLELSFIVAERLRKRRMASQRL

SEQ ID NO: 8 - protein, 254 aa, Nicotiana tabacum
MAASSTLSSSITTFKLSPCQPRASSTTASVKIPSIPPITLSILLISHFGPTPKNPTVAPLRCSATSTTPETTTTTSTPFHDLCYVVGDNIDNDQIIPAKYLTLVSSNPDEYKKLGSYALCGLPLSYQTRFVDPDEFSSKYSIIIGGENFGCGSSREHAPVALGAAGVAESYARIFFRNSVATGEVYPLESEVRICEECKTGDVVAVELAESRLINHMTGKEYKLKSIGDVGPVIEAGGIFAYARKAGMIPSREA

SEQ ID NO: 9 - DNA, 386 bp, Nicotiana tabacum
  source 1..386: mol_type = genomic DNA
atgggtctcaaaggcaagttgatctctcaaatggagatgaagtgtgctggagatttgcttcatgaacacttcaaatcaaatccacaccaaacctccaccatgtctcctgataagataacaaatttcacgttacatgagggtcagttgggtaatactggttctgttgtcagctggaagtatgttctcggaggaaaagagaggcatgcgaagcaggccctacacatagatgatgcaaaaaaatcaatcaccttcaattttcttgaaggttatatgaatgaattatacaagtccatgacaccacaatatagaatcaataataatctagaatgccataagtctcgaaaccatccaatgcaagttacaagtcctaaccatacgcaaatctg

SEQ ID NO: 10 - DNA, 1622 bp, Nicotiana tabacum
  source 1..1622: mol_type = genomic DNA
atgaaagcagaaggctcagcattatcatcagctggttcttatcatcgacttgcttatcatgaagttattaatgatgataatcaaaacaaaatttttacaagtgatgactctcgtctcagacaattgggttacaaacaagaactctatcgtggcctttcgttcattgcgaacttctcatttacattcgccattgtatcagttcttacgggcatatccacattgtataatcaggccttaacttttggtgggcctataactcttgtttacggttggcccatagttagcttaatgacacttattgtgggcctggccatggctgaaatatgttcagcttatccaacttcagctgggctttactattggagtgctaaattgtctggaaattacttcggcccatttgcttcttggattactggctggtttaacattgttggtcagtgggctgtcacggcaagtatagatttttccttggcgcagttagttcaggtgatgattctccttagcactggtggattaaatggaggtggataccaagcctctaaatacgttgttatcgcactccacggtggaattctgcttttacatgctatattaaacagtcttcctatctcatggttgtccttctttggacaactagccgctgcatggaatgttttaggtgtctttcttcttatgattttgatcccaatggtctcaacagaaagagccagcgctaaatttgtgtttactaatttcaatactgacaatggggatggaattaacaataacctctacatcttcgtcctcggacttcttatgagccagtatacgttgacaggttatgacgcttctgctcatatgacagaagaaacgaaaaatgcagataagaatgggccaaaaggaatagtaagtgctattggcatatcagttcttgctggctgggcttatatacttggtataaccttcgcagttacagatatcccgcatctattgaataaaaacaatgattctgggggttatgctattgctcaaatcttttacgatgcattcaagaatagatacggcagtggtgttggtggaatcatttgcttaggtgtaattgctattgccgtattcttttgtggtatgagctcactaactagcaactcgaggatggcttatgcattctccagagatggagcgatgccatattcgtcgttctggcataaagtaaacaagcaagaggttccactaaatgcagtctggatgtcggcctttatagcattttgcatggcattgacgtctcttggaagcttggtagcatttcaagccatgacatcgatagcaacaattgggctctatattgcttatgccttgccaatcctatttcgagtgactctagctcgaaagtctttcactccaggtccttttaacttgggaagctatgggctcgttgtaggttgggttgcaatattttgggttgcactcatttctgtactcttctctttgcctgttgcataccctattacagatcaaactctcaactatactcctgtcgcggtcggtggccttctcattcttgttgtttcttcttggatcttcagtgctatacattggtttaaaggtcctattaccaatttaggaaactctagtgaggaagcata

SEQ ID NO: 11 - DNA, 437 bp, Nicotiana tabacum
  source 1..437: mol_type = genomic DNA
atggcttctactcagcaagcggtgagttctggttctgatgcagaccagcggtatgcaaagtttgatgaacggaaaaggaagagaatggaatccaaccgtgagtctgctcgtaggtcacggatgaggaagcagcagcgattgggggagttgatgagcgaaacaacacagctacagaaccagaacagtatctgccgcgagaggattgattctgttgaaagaaattattgtgccatcgatgcagagaacaatgtgttgagggctcagattgctgaattgactgaacgtttgaattcactgaactcgctcactcaattttgggctgatgctactggatttcctgttgacctccctgaaattcccgacactttgcttgagccatggcagctgccttgccctattcaacctatcgatgcttcttctgatatgttgctgttttg

SEQ ID NO: 12 - DNA, 1965 bp, Nicotiana tabacum
  source 1..1965: mol_type = genomic DNA
atgccgggggtctatttggaaacagcctctctacccaagggtaggggtctggaatgtcaagaatcacaagctgttaggtatttctttcgagggagaaataaagtcgatgattcattgactattgagattttcaatcttttcccttggattttcttcaccatattggccatggacaagcaccaccatcaactacctctaaccaagtctacttcgcgccagcgttataacgaatgggtatttcgagatgttcctagtgatataacaatagaagtggatggtggcatattttcactccacaagtttccccttgtttcgagaagcggacgaatccggaggctagtagcagagcacagagattctgatatatcaagaattgagcttgttagtttaccaggtggaacagaatcattcgagctagcagccaaattctgttatggtgtcaactttgagatcacagcagcaaatgttgctcagctttgttgcgtatccgattatctcgagatgtcagaggactactcgaaaaacaatctcggttcaagagctgaagaatatcttgacagcattgtttgcaagaatcttgaaatgtgtgttgaagtcttgagacaatgtgaaaacttacttccacttgctgatgagctgaaagttgttagccggtgtatcgatgctgtagcatcgaaagcttgtgtcgagcaaatcgcctcaagtttctcgcgattggagtatagtatctcaggtggaagactacatatgagtaaacaagccaattgcgaattggactggtggattgaggatatttcaatgcttcgtatcgacttgtaccaacgtgtcataaccgcgatgaagtttcgtggggttaggcctgagagtattgctgcatcactagtgaactatgcacagaaggaattgatacaaaagaccctttctggttcaaatatccaagaaaaactagtggttgagacgatcgtgagcctgatgccagttgaaaaattcgtcgtgcccttgacctttctttttggattgttgcgaagtgcagtgatgttagattgcacggttgcttgtaggcttgatctcgagaggcggataggatctcaattggatacggctaccctggacgatatactgattccttcctttcgacatgctggtgatacattgtttgatgttgacacagtgcatcgaatattggttaacttttcacagcaagagggcgatagcgatgatgatatggaagatgtatcggtttttgaatccgatagccctactacgacgccatcacaaactgcattgttcaaagtatcaaagttggttgacaattacctagctgaaattgcactagatgcaaatctaaagctgaacaagttcattgctgttgcagaaacattaccagcacatgcgcgtactgtccacgatggactttatcgagcaatcgacctttacctcaaggctcatcaaactttatcagatccagacaagaggagactatgcaaattgattgatttccaaaagctctcacaggaagctggtgcacaggctgcacaaaatgagcgccttcccctccaatcaatcgttcaagttctttatttcgagcaattgaggctacgaaacgccttgttttgttcgtaccctgatgatgacattaagccaacgcaccagtcttggaggatcaatagtggtgctcttagtgctgcaatgtctcccaaggacaattatgcttcgttgagacgagaaaatagagagctaaaacttgaactagcgcggatgaggatgagattaaatgacctggaaaaagatcatgtttgtatgaagaggaatatgcaaaaatctagctcgagacgattcatgaaatccttctccaaaaggattggcaaaaagttcaatattttcggacataatttttccagggattgtagttctccctcaagtcagtcagaaagaactgaatctaaaataactgaaagaacttga

SEQ ID NO: 13 - DNA, 911 bp, Nicotiana tabacum
  source 1..911: mol_type = genomic DNA
atggagcattctgcagcagatagggatcctaaagctgtagaatttgcaaaggataagaatggagttggtcaagttttgcttcgaaatccacgtggcgcctctgttcgagttagcctgcatggaggacaggttctttcttggaagaatgaccatggtgaagaattactttttataagcagtaaggcaacttttaagccgccaacagctgtgagaggaggaattccaatttgttttccacagtttggaaaccggggctccctcgagcaacatggatttgccagaaataggatgtggatcattgatgataatcctcctcctctacaccctaatgattccaatggcaaagcattcaccgatttactacttaaatcatctgatgatgatcttaaagtctggcctcatggttttgaatttcggctgagagtaactttggctgttgatggatctcttaccctgatatcacgcatcagaaatgtcaactgcaagccgtttagtttctccattgcataccatacatattttgctctctcagatatcagtgaagtgagagtggaaggcttggagactcttgactaccttgacaacttgtgcaacagagaacgtttcactgagcaaggagatgccttaacatttgaaaccgaggtggatcgagtttatcttagttcatcagatgtgatagcaatttttgatcacgagaaaaagcggacttttgtgataaagagggaagggcttcctgatgttgtggtttggaatccatgggagaagaaatctaaaaccatagcagattttggagatgacgagtacagacatatgctttgtgtagacggagcagcaattgagaaaccaatcaccttgaagccaggtgaagaatggactggaaggttggaactgtccgtcatgccttcaagttg

SEQ ID NO: 14 - DNA, 1736 bp, Nicotiana tabacum
  source 1..1736: mol_type = genomic DNA
atggaagtaatgaagaagaaagaaaacacctctagaaaaatgaagggtggaatgattaccatgcccttcatatttgcaaatgagatatgtgagaagttggcagtggtgggatttggtgcaaatatgataatatacttgacaaatgagctccatcttccattgactaaagcagctaatactcttacaaactttggtggcactgcaagtttgactccattacttggagctttcattgctgatacctttgcaggaaggttttggaccataacaattgcttctatcatctaccaaatcggtatgatcattttaacagtatcagcaatacttcctcaactaaggccaccttcttgcaaaggtgatgaattttgcaaagaagcaaattctggccaactagccattctctatatatcattactcctaacagcatttggatcaggaggaattaggccttgtgttgtagcatttggagcagaacaatttgatgaaactgatccaaatcaaaaaacacaaacatggaaattcttcaattggtattatttcagtatgggattttccatgctaatagctgtgacagtaattgtttatatccaagataatattggatggggtataggatttggagtcccaactattgctatgcttatttcaattattgttttcatatttggataccctttatatagaaacttggatcctgctggtagtccttttactaggctattgcaagtttgtgttgctgcttacaagaaaagaaaattggacatggtttctgatcctagtttcttgtaccaaaatgaagagcttgattctgctatttctactgctggcaagcttgttcacactaagcaaatgaagttcttggacagagcagcaatagtgacagaggaagacaatcgaaaatctccgaatctatggaggctaaacacagttcatcgcgtagaagagctaaaatcgatcataagaatgggaccaatatgggcatctggaataattctaatcacagcatatgctcaacaacacacattctcagttcaacaggcaaaaacaatggacagacacttaataaattccttcgaaatcccagctgcatcaatgacagtcttcacattaacagcaatgttatgcaccatttgcttctatgaccgcgtatttgtgcctatagcacgtaaattcactggtctagaacgaggcatatcgtttcttagcagaatggctattgggttctctatttcagttctagccacattagtagctggatttatagaagttaaacgaaaagaagcagccttaactcatggactgatcgataaaggtaaggcgattgttcccatttcagtattttggcttgtgcctcagtattgtttacatggtgtggtggcatttatgtcaattggacatcttgaatttttctatgatcaagcaccagagagtatgagaagtacagctactgcattattttggacatcaatttcagctgggaattatttgagtacacttttggtttcattagtgcataaatttacttcaggatctggaggatcaaattggttacctgataataatttgaataagggaaaattagagtatttttattggttaatcacaattctacaagtggttaacttgatttactatctgttttgtgcaaaattctatacttttaagcctattcaggtacacaagacagaagatttggactctaaaaaagatagtattgaacttgtaaataatgttta

SEQ ID NO: 15 - DNA, 1553 bp, Nicotiana tabacum
  source 1..1553: mol_type = genomic DNA
atggctttatcaaacaccttatcattgtcatcatcaaaatcccttgttcaatctcaccttctccacaatccctccttaccccagcctcgtattcccgtttttcacaacccccaacatgggcggcgccaccccatctccgccgtacacgcggcggagcccgccaaaacagcaactgcttcacagccgttgaaaaaaacccaatggagtcttgattcttggaaaagcaaaaaggctttgcaattacctgaatacccagatgaaaaagaacttgaatctgtgcttgaaactcttgaatctaatcctccacttgtgtttgctggtgaagctaggaatttagaagagagacttggtgaagctgctttaggaaaagcttttttattacaaggtggtgattgtgctgagagttttaaggaatttaatgctaataatattcgtgatacttttaggattcttcttcagatgagtgttgttcttatgtttggtggtcaagttcctgtgattaaggttggaagaatggcgggtcagtttgcgaaaccaagatcagatccgtttgaggagattgatggagtgaagctgccaagttacaagggtgataacattaatggcgatacatttgatgagaagtcaagaattccagaccctcataggcttattagggcttacatgcaatctgctgcgactcttaaccttcttagggcttttgctactggaggttatgctgcaatgcagagggtcaccgaatggaatcttgattttgtggagaacagtgagcaaggagataggtatcaagaactagctcacagagtcgatgaagccttgggattcatggctgctgctggactcacagtagaccaccctatcatggcaacaactgatttttggacatctcacgagtgcttgcttcttccttatgaacaagcacttacaagggaggattcaacttctggtcttttctatgattgttccgctcacatgatttgggttggggaacgaaccaggcaacttgacggtgctcatgttgagttcttgagaggagtagcaaacccacttggcataaaggtgagccaaaagatggatccaaatgagctcgttaaactcattgacatcctgaacccaaccaataagcccggaagaattactgtaattgtgagaatgggtgctgagaatatgagagtgaagctttgccacttgatcagggcagttcgaggagctggacagattgttacctgggtttgtgacccgatgcacggcaacaccataaaggcaccatgcggactcaaaacccgtgctttcgattcaatcctggctgaggtccgagctttcttcgatgtgcatgagcaagaagggagccaccctggtggtatccatctagaaatgacagggcaaaatgtgactgaatgcattggcggatcacgaacagtaacctacgacgatttgggctctcgctaccacacacattgtgacccaagattgaacgcttctcaatctctagaactttccttcatcgtagctgaacgactaagaaaacgaagaatggcctctcaacgtctgta

SEQ ID NO: 16 - DNA, 764 bp, Nicotiana tabacum
  source 1..764: mol_type = genomic DNA
atggcggcttcatcaactctttccagttccatcaccaccttcaaactgtctccgtgccaaccacgcgcctcctctactaccgcctccgtcaaaatcccttcaattcctccaataaccctttcaatccttttaatttctcattttggcccaactcccaaaaaccctaccgtcgcaccactccgttgttccgctacctccaccacaccagaaactaccacaacgacttccacaccattccacgacctttgctacgtcgtcggggacaacatcgacaatgaccaaatcatccctgcaaaatacctaaccctagtttcgtcaaacccagacgagtacaaaaaactcgggtcctacgcgctgtgcggactccctttatcataccaaacccgtttcgtcgacccagatgaattctcatccaagtactccatcatcataggcggtgaaaacttcgggtgcgggtcgtcgcgggagcacgcgccggttgctttaggagctgcgggtgtggcggagtcgtacgcgaggatattcttcaggaactcggttgcgactggcgaagtttatccccttgaatcagaagtgaggatttgtgaggagtgtaagacgggtgatgtggtggctgttgaactagcagagagtaggttgattaatcatatgactgggaaagagtataaattgaagtcaattggtgatgttggtcctgtcattgaagctggtggcatttttgcttatgcaagaaaggctggaatgattccttcccgagaagctta

SEQ ID NO: 17 - DNA, 2500 bp, Nicotiana tabacum
  source 1..2500: mol_type = genomic DNA
tctgacatatctatagcatttcatttcactacaagtcacttcatgtcgtcaccgtttaatgtgcttaatatgtcgaaaccatgactagtgatatagacattctcaaatttcttaaagaggcataaggaacttttgcatggattgaaagagagcatatataatttatttctagcatccattgcctaaaatgagatgactctctctccactttaatttccatacaatctcaaggaagacattggatagtagatatataagctccattttgtgaactcctgtaagatcattgcctcatgtgctatattgcacggactttccgaaatgctgttgtattcatgtcagatcctctaaaaatatattacttttggagaatctgacacacatctagagacattttcggaaaatctgagcaatgtagctctagtgtgccggagaaagcataggtcaattagtcttgcaattgactaattgcaatctgctaatagaagtatatatgtaagagtacatcgacatggtaagcactaaactatcagataggctatcttttttcatatacctaagtcttggtgcacaaactcggtacttatgctggtgagaagtaatacctatccagtgaaatagtcgaggtgccggaaaatcagtctacgcactacttagttaaaaaagttctaaatcatctaaaaggagcaggagcttttgggaaagaaacaggtataaggcaatgtgcaatggtgactgtcgacaacattaattatgcaaaggtgagaaatatcatctctttattgtaaatataaagtgacacacacgagcagtatggaaaatctaataaagttgatgcttgtgcaacaaattcatttggaatttctttggtgaattattcttttggttgtcaatgtgtaagtgtttagagtagacatgaattgataactgaagaaaagtttgagtaacttgttggaccctgccaaattattgtgaatgtgtaagtatttccacaatgtcactgatcatactgttgcagatacacatacggccagaattcaataatagcaataactaaaaatttctaccaaactgaaaatgcaaaattgaggcagataaaatttgtaagattgtggtatggggcgtgaagttgttgacttatagccactggtgcaattgatttaagataggaccttatctcttctcatccactacctttttcgtttgcctttcatttaccttgctccattcattttctttatgtatatccagattttttaatttgaatttgcagttcgtttaagtataacttcagcagctgctgacacatgtcacgttagttacctcttttatttgtgggatgtggcgagcagtgatctgataagggatatttgacctttatcgaacacatgacatgaaaaaaaaaaggttaattgatttagttgaagataagtgaagctctaaaggcaattgaaggaatttaaatttactaaaatccaaaaacacgatattaattatatgttccgtgcatgcttaactcacgcgtactagagattttaatccttctaattctattacgtttattctaatacttgcattggatttttactagccaaaactcgacgcagattgatctccttattctcactaaagataagaggagccaaaagtggagtaagaaatctttacaaactaaaggggttagatgaaaaggaagaatccaatacttcggattcaacatgttaacaaaagcaattttttaaccgactttgtcactacagggaaaaaaaatatttttacactattcaggcagtctaaattttcaacgaacggaattcaattaaactctctacgtcacatgtacatctgtgatcagcaataattgttgagttgatttggaattaagtaaattagttctgatcactccggaagtgaaactgcaaatgtgcaataacacgaacaaaagactatgattccatggtttcaacatagcccacaaacaagctaattgagattatggcgattaatagaagatcattaggtttaatttggcgaggcgaatagccattgcatgtatagtccaaaccatgaaaatgacacaacaaaccatacgacaacctaaaaaagaatgcaaatataggtggaacttgatattgaggattaaataactgccccacaagaaaaaacaacatgccccatattcaaccataacgatcactcgtatttaattattctctcctttaattcaaataaaaaaaaatcttaggttcttggcttcttagccatatgcttttagttccaggcaaaagtactgcaacattcaatctccaacgtacaaactctcatctacaatggattgaagctctcatttgcttagcaatcactttcaactctgttataatactgttatatgtaagagacccgacccatgatcaagacccattccacaactatataatatatactagtgagtgaggagtaataaaagcaagcaaaagctaaaaggaagttcttgagccaa

SEQ ID NO: 18 - DNA, 1406 bp, Nicotiana tabacum
  source 1..1406: mol_type = genomic DNA
ccttatagaaattgtctgcttctttcttctcgaaactcaaaaccccgatcgaaaaattagtttttggggttagaacatggatgggattgagtttaaaaacgtaaagatcatagagcctagagggaaaagaatctgatgtcatgtcgtaaaggaaaaggaaagcaaataacagtgtccagcctcgaggcaaaggaagaaaagatgccccggaaacaggaaggctttaggctcaaattaagcacactcaattcactcctctgtttaattagaatttccatgtttcctccttccgcggacacccccatcttagtttcttccacaacatttataattcaatgtcctaaatttggaagcgacatattgcattatacctctagtaatcagttggattagccgattaggtaaacgtaagagatacactatgtaagtatattcttcaaggtatatgagaatatgtttacgtaacagaatgatttaaatgaactgattattgattctgtattatcatgcttatgctttcgatgattaattagccacatctagctaaactttttcttccatatctttttttttttttttgtgatttacaagaatagatcgtgtcatttgttttcttaaagtacctatgtcaacatcatggatgacatggaataacaagtgcatggaggtcgataagaaaaaaacaagcagctccttggatttttcgaagtttggactttttagagcctcaattttgctgaaatatcgaaatttggagacgtgagattcacccagtcaccgcttacgatttaatttatatacactgacaagtataaataaattataatattataaatttaatttgacagttatatcatgttatatgctttatttttcgagtaactccttcaacttattgtgagcattacctatggttattttgaatttaaggtattacattgacgctttaaagttctattacacagtagtgtatagaagttaaactcagactgttttcaccctttgttcacactccaattttgaaaaactgattaacaaggactatccaacttctatttaagaagaaactaatccttgtttgatttaatcaatcttgaatttctgtgaacggtacaaattccgtgaccaaactggaccatcatctaaactccaactaacgcacggaataatatcaagctttatgctttagattttgtattttctaacataaagatcctagagaaacagaccacataaaagacattttacgacgtgcatgtccagcagtggctataaaaatgataagaaatgtgcgctcaatagtcgttccatgctctgtttatatatatgtaccttctcatacattaaacatcatatcatacaaaagatcactaaaacagaaggaaaaaagaaaagatcttcagaat

SEQ ID NO: 19 - DNA, 2500 bp, Nicotiana tabacum
  source 1..2500: mol_type = genomic DNA
gcgcaatatccaggacggtggtcttgcaccgcatcccacccgggagggatcaactcgacgtgatcgggaaggccgtatttagccttaagctctaataaatcagcctccttcattgccgacttaaaggtttcgggttcgacttcaggcaattttgagaaatcggacctggatcgtacgaggaactatctcttccatcgtcaggaagcttctgtcctcaacagcagtgggaatgctctcagcatgaggaggaaaaactatcgctagagggaccgggtcacttccctcgctggactcagaaggtacattagacatgttttaaacgaaagagaggaaggtatcaaacaacggaggattaaaaacgatgaggatcgctggaacaaagggaaaagatgcacaacaatggaaaaggaagaagaagatataaggtttcgatgtaggaagtctgtaaagtttgggattacaccctcatatccctatttataggaattcaggcgccagaaccaagaaattggctcatcattacctagcatcggaattgaagtggcaggaccaatcaggagccccatgcaaaataaagcgacgcatcgggaatacgcatcatgatgatgcacgaggtcatgacgtcaccttgattcatggacaacacaactccaaaatttgcagctcacgaagggccacttttctagctcgcctcaatcatcacgagccgatcagctcgtctaatcgttttgaccgtaatgaacataattcgctcatcaggcccgcctgaggtcggcctcaataagcggaggggctaactgtatgggtcaaaatctgtcctaaaatatttaagataagataatactaaagaaagaattctcgagccgtcgttagtcgaggtggactaggaaggagcgaaacttatagtcgaagaatcgacgagagccatggtcgagatgtcaataatggtcgaagtcgagcaccgttgataaagctgtaacaactagttttcgaaataggatattaaagagaatattctagtggattctctgcacttgtactattaaggtttactaggaatatgtctcatataaatagaaaaagagacaatgatatgaggcatgtgatattcatttgtaacaagatactttgacaaaaaagattctctctctctctctctctctcactaagatacaaacaccaccttttcactaagattcttgtctgtattattccatactttttcatcagatccgagaataattcaagcattcaaggatttgtgtgtcactcatcattgtcaagaggaacaaccatcccgttcatcctttattgggtgaatcattcctcctatttacttaagtgtcatttattgttattcattgccattaaatgccacattattattcatgatttttggaatagttattgcatactgttatcactattcgaccaaatctatgtgactttatcacacccttggaagctacgtctagaaatattattgttaactaattttaacccataatcacataaatttgattatttgaaccgagagtcatatttttggtcaaacatatactttattaataagttatcataatcgctttgtttttaaaggattgcacgccgaaatttgtcgctaataattgacatatctaaaacgttgtgtccccgttgcactcaaattccgggtccgcctctagacctactaacatttttaccactatagtgagtatcttgcatgtttcacgtacattttgtgcatctgcacactcactaatctttcttaaaatatgtgacgacaaatacattctttcttagaagcttgaagacagcaggatttctctgagataaaatgaaactaaagaaaaagaaaaagcaatgctttgggcttaggctcattcagaatatagagtcagtaaattcaaaatgaatatgatcttaatacatttattattttaattattttagaacatacgtaaacaatatatataataagttcaattgaactaagaaaacttgtctctctatttgtgtatgccaatattgaacgagactaccatcatgaaataaaaaaatccagtccactcgtatgtcgtatgactatttaactctaatgcttcttttcatagatattcatatgattcatgtcgaaaatataatgttcgatcggttgaattcttagctgacaatagcagacatttgaaaatgtatactccccttagttattccaacttccaaaagtcactaaactctaatagcatgacaggaagcgtaacaatgccatgccaaaatccaaattgcctagtaggagtaaaaacaaaatgaaatgggtcaccattcatcggcaattaggtaaccatatctcttttgatatttgagccacaaatatacgagtgatactcattattttaatctcatccacgtggcaccatcccattaactgcttatctcaggctgaaaccttagtatcttgcatattttcttcttcctcagtgtaaaaccttaattcacaata

SEQ ID NO: 20 - DNA, 2500 bp, Nicotiana tabacum
  source 1..2500: mol_type = genomic DNA
gaacacatctaaacaacttggaatcacctcaaatatcgcacagaagttgcaaatgacataacaaacctattttaactcctagaacaataatccaaactcgataacatcaaagtcaactcccgatcaaacctatgaacttttcaaaccttcaaattgccaacttttaccaattatagccaaaaccttctagaaatattcaaatgtaaatctgggcatacatccaagtccaaaattgccatacggacctaccagaaccatcaaaactcagatccatggtcaaatacataaaagtcaaacttggtcaattcttccgacttaaaactttctatctaagaatcattctctcgaatcaattccgaaccgcttaaaaactaaaatcgacgatatccgtaggtcataataaatcatacgaagctactcgtgccctcaaaccaccgaacgaagcacaaatgctcaaaatcactacttgggtcgttacatttttgagttggtcacactatttatttttgtacttgttgaattgatgaaactatataggttatagcaagttttacttatatttgttgcctcttttacctcgccgagggtagttatgatacttgctgagtacgttgggtcggttgtactgatactacactctgcacttaattgtacaaatccaggtgtcagacccagacattagtagctgaggctagcagaagagttgattgctgagcgacgaggtagaactgcattcttgatcgcagtcttggcgtctcttttcttaattactgttgtctttatttcagacagtattgtacttggtcatttcagactttatttccgtattagagcttatgactctgtatttaccagtttctacgggatatatcatgtattagcggtattttgctatattgaagttttagacatatgttatttctataaattatgttattttggttctttattgttatgtccggcttgcctagcaaatgtgttaggcgtcatcatgactggttgggattttgggtcgtgacacatatcttctctaaatgctatcaccgtaatgtatacatgtcttttattcatatcttctctatatgttgctttacatgatgaaataattcacggtatatatcttctctatatattaatctgtattacctgacttatttatccatgcatatcttttctatatgcaactgttgactggttctttgaactgttagtttgtttacaatgcttgtgcatgtcttgtctatatatatcgatggccttacttgtacttcatgcttagattttgttattgttcttgtgcacatgtacattcatgaacatttcaggtttcataaaattagtatcttttgacttaaattctcattactacttcactgagattagtcaagagatttactaagtacatgtggttagttatactcatactatacttttgcaccttgcgtgcagatttcagagttgagctgctgtgatgatgaaggccagcattgaagaggtaccggtgttctagatacaagttgtcacttgttcatggttgtttacgttttatattatatttatgtaaattttaaatagatgctgtaatctctgttcatattagagttgcactcgtaatcttgttcttaatcgttcatgacttgtactaccagtccttgggataattatgtgaattttctcaatcttatttattatttattgataatctttcattcgagttgtgttatttgttgtttggcttacctagcataccatagttaggtgccagtactcgtaacatttgttcttactttctagacttttagtgtatattaaaaatatttatttttatgtactgctttgcattagtaagaggatcttttaggtataatgctctttcttacttttaataactatcgtcttgtattaataaaattttcttgcaaattttaaaagcaatgacatattaatggagaaattatacacaatataaagatataatcaatttaaaaaagcaacatgtaaaaagttgtagacgagataagatgcctattatttatatttggtacatgaatgtcattttccatgaaagtttttcaaaaccaataatttgtatatgttagttgaaatgtcacttttgttttgttcaatcagaaccagtaatttgtatttaataatactaatgtcatttttatccgactcaattaactactccctcttttacaacaaggaaagaaaagcttgttactctccctataagtttttcacgaaggcaatacaattgttgcaacttctttttcataatcgtagtttttcttcattgatcataatgggtggaaagagttctccaacgatgttttgtttggttctagccttcttactttatgcatctatttagtataattttattatatataaatgttcggatttttctctcactactttctcccccttacttcaacccaactatcaaaagagattatttttagatatcaaaataattaacacaaactttattacaaat

SEQ ID NO: 21 - DNA, 2500 bp, Nicotiana tabacum
  source 1..2500: mol_type = genomic DNA
caaaatactctataatgaaataaatgaggagaaagaaagagaagagtgaaaagtcttgaattggtgtgtttactaatgaggagaaactcctctatttatagcaagaaatccttagcctaataatggatattatgtcatgcaaatgtcatgatctacaaatttgttataatggatattatgtcatggcaaatgtcatggaccaaaatttgttataatggatattatgtcatggcaaatgtcatgaaccacaaatttgttataatggtattatatcatggcaaatgtcatgaaaatttggccatattacataggatcacttttcgaagacaaacacaattatatggataatacaaatgatgtaaattcatcgtgtttaaagcatattccatttcggatgcctccaaatctgaaaatatgtaacgccaaatattgtttattttaaaaaatgccaattatacaaggctctatccatgcaaagtaacttatccacacgtaaaagcaatacaacgttaattaggccaagaattagcataagttagtgcaattttaaagttttgtactcaccttttatgcctcaaatatctgtccaaataaagtagtaaaatttaacaaattgatatcacagagaaacaaagaaattatttggaatcattctagtgtactttcaaatattcactaaatttatctcgaagttatgcataatttgcgcaccttgagctaaaataattgatttgtttctattatatacgtttatacattaccagtgctttttgtttttttggtctacttgttcggcacaattattacacttaacaatgcaataataattgagaatacggtaaagggccaaaattatccctagactattcgatttggtataaaattgtcctccgttcatctattgagtcaaaaatgtctatattgttattttagtggctcaataatgcctttattactaaccgacttatttgaaaaaaataattaataaatatccacgtgtcaccgtccattggctaaataaaatacttactaaacttttaaaaaagaatcaccataacctaggttttgattggcgccgcacgattataaacctgtaatccaaatcttttattcttaatagacaatgtaacttattcttcttcctcttttaattaagttcagttctgcaaatgggagaatttatgtcaccacaacaacaagaaaagtaacaatttaagcaagaaaatgttgattttgggaataaatgaagtgggtatgggaataaaatttcggatcttgacttaggaagtaattcaattaaatgagagtaaattcattaatgaaaacaaatgggtatgacatgggtgaaaataattcattatctcctcctctaattcgatttcatggagatgggagaagaaagagggagttggcagctaggcgggtcctttttggggccggtccgatgaaaacccaggttatggttttcttcttttttaaaaaaatataaattggtaaggatttatatttagaccaatgggacggtgatatgtggatattaattaatttttttagataagttcgttggtaataaggacataattgaaccactaaataacggtaaggacatttctagctcaataggtagacggggatatttttgcaccaaatcgaatagtgtagggtagttttgacccttctccgtactagaagcaaggagattagtaacatagcaaaattattttgttaataccaaatcaaacaaagcgaccaacatcacagttgaggaccacggcgccttagttcatcatattctatgatgataactgttttaatactaataaataagtatgattataattgtttaaacaataaagaaaaccaagaatatgtccagttacgttataatgataagactctaccaaacaacacatgtcaacttcatcgctaatttgaattgctcatagctaacaaaatttttgataatttatcgtaaatctataactaatttggattagcgacgattttgttcagctacaaaatttgtccgtagctaattcctgttttttagtagtaaaactagcctagtacaactttgttggttgtgtgacataaaaaataaaatttctcgtattcaactaattaatcacattccaaattcttcataaatactaaatagattctctttttccataactgaagcaccaattactcgagaaaagaaaattaaagtaaatatgggttccgaaactatcaagcttcctaatatagacttctccaatgtgacctaaagccaggcacacttgtatggaaccaagtgaaaagccaagtccacaaagctctagtaaactatggcgttttgaagcatcatttgataaaatcctatacaccttcgaaaatcctttttgaatccttaaaagagcttttcgatctccctttacaaaccaaaataagaaacatttcaaccaaacctttccatggctacgttggacagtatccagcagttccactctatgaaagt

SEQ ID NO: 22 - DNA, 2500 bp, Nicotiana tabacum
  source 1..2500: mol_type = genomic DNA
ttaaggatatgttttaaataattccttaaaaaaaatactctgagcattattggtactttaagcttacaaaaaattatacttttatttttcatcaaatatttaacaaacgctatttgtcgaaattgtggggggaaaaaaagaatttaactagtaacgctagaaaagttccataaaatttcagaaactacagtgcaaaattgcactagacacaaaaaggtaaactaaaaatagcaaaacttgcatctcataaattgcaccaaattctagaaaatagaccaaaaatagaagaactgtaaaaattgtacctttaaatgtgagttctagctaaattctttgtatttcacagttccctcaaagacggagtttgtttactatttgacaatgaaagaaaatagtaaaaatagtacgatatagtcagttttcggactggtcattcaaaaatagtcagcgtttaccaagtcaataaaaatagccactattttgctgcaaaagagaccgatccagcataatatactggagttcggtgcacctgtgtatgaactccagcatattatgctggaccgatatactttgctggctccagtataatatactggagactggagcaccggtgctccaaactccagtatattatgatggaccggtatacttgctggaactccagtatattatgctggagttctagtgtacttatgttggaactccatcatattatgctggagttccggcatacttatctcggaactccagtataatatgctggagttcaagtatacttatgctggaactccagcataatatactgacgtattttccgggttttgaacagtattttcgctcagatttatctttacatgaaaagtggctaaatttcgattacttttgaaactgggctatttttgaacgaccagttgtaaatctggctatttttgaatttctcccaagaaaatatcctaacttaaataacaaaaaatgctcgaaaggtatatttgaagtactacttttttgaacctattccaaacttacaagtaggggtgtacaaacggaaccggaaaatcgcaccaaactgaaaagtcaaaccaaaccgattaaaagacccgactaggtttggtttgatttggtttggtattgagtaaaaaaatcataaccaaactgacatataaatatataatttttatatatacttttaagattttatataaaattttctttaaagaatatctaaaatatttgggattctcttgtgggatataatatttaatatgatccataattattaaccttaaataatgggttatatgatcgcgttctcatcaagtgttactgaaatgcgtcaatctctatgtccgtccatattcatatcatatgttaagatctattatattcttatatcttttttcgaatgtgaagtgataattagtattatttaggtatcatattggtttttatatttaattactaattcggttaaccttgaaaatatatatcaacaaaaaattattgtcaaacgaataaaaaaaataactattatgtgttactaagaaaattctcccttaaaaatattttaatagataatttgtcaattttttatatttttactaaacatatatttacttatcaaaaatttaataaagtaaaattaaaataatatttaattaacaaaaaacctgaaaaatcgaaaaaatccgacaaaacaaaatcaatccaaaccgatagggttgggttggtacacccctacttacaagctcatctctattattttctcaagtttgtattgaaggttcaaatttcaaggattataattataatgattgcaaaacgacaatcttaatataggtgattaattctgatatggacaaaattttgcgtcgtatccatgatcgaccaactaccaataattttcaaatgctcaagggtttcaagtgcgtgtttatgttctgaaattgtagctagttggatatattccatcaaatcttgtaaagctaataatgtttcttgacctttttaaatatactagtgatcctttcatgagatattgagatcagttttttctatctgcttggaattgaataatatagaatataaacatatgatatattgggggcaaatacggacataaaatttaaagtgagtcacttactttatatgttaaactatgtcaagattaatgaccacaatccgaagccgaaggaatataatacgaacgatgaattttgtttacgattaggtggaatgattgatattaaaaaagaatgtaaaagcaaataagaaaaggtgaataatgcatattaacaatctatattttatacgattaggtaggaagataatatgattgatatttaaacgtaaatattttaaagtacgttgctgtctttgtgacccctctggccataccattacccattatttatattccctcgtatcaacattcagtagcaagtaaaaagagataatttttgttgattatttgttgttgaacttgataaattttgctgaaaaa

SEQ ID NO: 23 - DNA, 2500 bp, Nicotiana tabacum
  source 1..2500: mol_type = genomic DNA
gagacgcatattgtaagaagaagaaagaggggataagataggtgggtcgaggaccgggagactgcatatgtaagctttcaattttctttaagtattataatttacttttataaaattttaaatactatttaatttaaaataatatagggtcgctaccaaagtaacgtggaggaattcatcgtagtcaccagctggtgaatcgggtgagccaacccaaccttcggacgaggatgctgaaagaatatggttggagtctgttggcggtccaaaatggggaaggtatacgggcttcctactaaaaattccatcgctataagtgtggaatgcaggaatagggacttcctgcaggcgagaacttaatagggagagcctctcgctatgcgggagacagtacaaagctacatcgagctagaagcggccaaggaaagagaaaggcttagagatgctcaattccttggcatgcaagctcagatcagaactctcctatctatggagcttttccgttgcccgtctcgtgagtcatcccagggtcgactccacgtgatcgttcctcctccttgtgatcgttcctccgtcctccccgtgatcgtttcccgtcctccacaagaattctctatacgtcttgtagatgaaagttcatcagatggtgatgatgttgtagaaaataccccttgaccaatactttgatatactttgaactagactaatagaactgttttgaattagattgaacaattttgaacttgttgtaattagtttttgcttggttttggattgtgaggttcaagtgaactttaatttgttagttttaaggtattaattggatgtttggttgttgttgttggatatttagttagatttgtggtgaattaggggttgtatatggtgaattaggggttattagatgtgaattgggggtattggtatgctgtttttatttgacaggtggtatagctaccaaaacaggcattttctgccaaaattaaacccagaaaaccgaccaacatttgtcagtaactaaaaaaaaaaaattaaattgcacattcacaaccaatgttggttggttaatgtacaatgaatttccagaaattcaacattaccgaccgaatttagtcggcttgttgcctgccctgtccagtttaccgaccaattttggtcggtatttttaaattttaattatttataaaaaaaatatattttccaataccgaccaaagttggtcggtatttttaaattttaattatttataaaaataatatattttccaatactgaccaaaggcaaaattaataaatttaatacaccgaccaagtttggttggtaaaattaaattaataaataatattccgactaattttgatcggtaagtcaattaaattgtcagatagtcgtgtagagcataccgaccaacattggtcggtaatttccgagtaactttggtcgctatgcccttccgatcttcaaaatactgttcacacgtgaatgatcgcgttttgaacggttattgacctttaccgacttactttgatcgtttttttggacgatatttttcgaatttctaatagtgactgtcatctatttctttttgaaaaaagaaaaaaagaatcaaacttgagcttggaagcacgggggaaataacatggcgatttcttttgtcataaaagaagacaacaaacaactcctcgaattctgcttttaactttttaagttgacttcccaaatcccaactactcaggaaacagttgagcttgtttgcctagatctaacacaatatataatgcatacgaccccataacaaaatctgtattttcacgtttattacactgaaaaaaaaaagataaaacattatatagcagacaagataaaaccttctttgtcaaaacaaaatacaaaatatgtactaagagaaaatcaccaacatatcgttacggattcacttgtttgataaagatgatcttcttttgtggatatttacataaataattgatcgaatttattgtttatacataaattatacactgattatataagggtatataactattatacatccgtcgactagttcgtttcagcaattagttgaacaactacttaaattaattctttttttaatataagtattgaaattcaattgtcttcattcttttataacagtaaaattaatgctcataattttgtgagccttacattaattaaggaccgcgtagtcgtaaaccaactataaaatgaatataataagctgtaaatttatctacagcatttttagttagcgtgcgtacgtaaaaattcaaatcgagagacaaacttataacttactacaacctaattatatacgaaatgaactaacactaaccttcgtccccactaattaaaaatcgtggatcatgttttatactagctttgcatacagctaattaaaatctatatagactacaacttttgaacattttctcacaccactttcttttcacaacttttagctactccgaaaaaagtaatt

SEQ ID NO: 24 - DNA, 2500 bp, Nicotiana tabacum
  source 1..2500: mol_type = genomic DNA
ctgaccataaaagttttctaatgacactattccaataactcaagaccagaaccaacttaaattacattgtaattttacggatgattattcaacttttactttgttacatcgaagtcactaaatttttgaaactttgctactttcttcctcctttattcgaggtagaagaagacagtgcaatagttgacttattagacagctttactcatatcacgagagcttaaaggagctttgtctaattccatgatcttaatctgtccatatgtccttgtatttgattttcaagaaaggaattctttttatttcagaggcggatccagaattttaattttatggattcaggattatattagagcctattttagtaatacagttagagtctgcagtaccatttgaaaagtctcattttaaggcttttcactttgaaattccttatcccatttttcaagtttttgcttctggagccactccttttctgagcctgcaagttcaagtgagcgttactgcagttcaagaatgacaaaaaagggttgtgataagctgcaatggattgaagtgaacgtcaaataattaatgctgtgactattgcaacttttgcagcttctttaggctttgtctgaagtgaatgtcttcattcgaaatgaatagacttcagcaagctatgttgctaagattctccgaaaatgtcacgaggtccatgtcagatccttcaaaagtaatgcatttttgaaggatctgatacgggtgcggcaacattttggagagtacgcgcaacatagccagccaacaagtcgatacttacggtctaatagtttgaagatctcctatccaaccaagattattgaacatgtttcgattgccatttagctcaaatgacaaaatgaatgaatatttcctttttaagtacacgtctctaatacatttatatagtcactccggaaaccctgttacatatattagtatctacatataggttaattggaagccactgaaacaaccttaatactttaaaagctaataaatctacagaaaagagctaaaactacccctaacctattcgcttaggtttaaaaatatccttcgtccacctattttgtaaaaattgcccctaacatcaacttttcggcccactcataccctagaaactaacgaccccattttgattaagataatttttttattacttattatgtgtcaatttcctaatggcttaaattaaaacctcattccacatcctattagcccgctccataacccaaaatatccatactcgacccatgaccggctttttaaaatgccttaacttcacctctcatcttctcctctttcatccctaccctcccactctatattttctctaatcatcctcctccatggccaaagtacaataactcgccattggtaactgaggactaccgtcggcttcaactttaagatcttcacccttcaacacccacggatcttcctctcccactcagatttttcggccttaatttaatttttaaattaatatttttttgttaaatcaattttttgagtgaaatctggtattctttggaattaaattaatggttcatttaaagattttatggtttaataagttatgtttccgccaaagttatattttttgtaccaccatgtagcttttttggagttcgacggcggtgatttggttaggaaactgttggcggagattaagttgggaaagaaaaacagggaaagggataaggtaccaaataataaattaataaaaagaaaaatatgaaattttgatatatgaaaccaaaataacgattggatcatgagtccggtatggaatcgggtgtttgaggggctaaaaagggattaacgagttggaacgactaagatggattgcgagtggcttatttacggatcagttctgggttagagtgagatttttaattttgaccaataagcaattgccacatcatatataaccaaaaaaattttattcagcaaaggtccgtcagaaataagggcatgaatgagccaaatttttaacgatgaagacagtttttacaaaataggtggacagatggtatttttaaacccaaacgaataggttaggggcagttttggcccttttttgataaatctaatatcactccagtaatgacaaagctagaatctacatatatctgtctttattttcaacatatagattccttacacatcattgtcacaaatcttgcttaaatggaatctcagagttaaatagagatagatttcactcaattatcagatcataactaaactacctaaagatggatttaggaggaattatcttaatcttccttttcagtaccctctgcctctatctcctttggacaatcattaaactcctttatttaatatggtggatgccacttcaaatacaaaatagaatgagttttcagggaatcaaaggccccccttatagctttccccatgggaataccaaagaaatctcactaatgagaagccaaactatggacaacct

SEQ ID NO: 25 - protein, 318 aa, Nicotiana tabacum
MCVYSNCNKKRGRGRERGVVIQGKTAVGNNKTTNYYLYFLIFAPSLTSTAHRFSDPKKVAKMNDADVSKQIQQMVRFIRQEAEEKAYGFPSPPKKYWPISYSIDQLALVRLILSNNVRIEEEFNIEKLQLVELEKKKIRQEYERKEKQVDVRKKIEYSMQLNASRIKVLQAQDDLVNSMKEAASKELLNVSHHQNHHIYKKLLQDLIVQSLLRLKEPCVLLRCREDDVSLVEGVLDAAKEEYAEKAQVHSPEVIIDQIYLPSAPSHHNAHGSSCYGGVVLASRDGKIVCENTLDARLEVVFRKKLPEIRKCLFGQVAA

SEQ ID NO: 26 - protein, 659 aa, Nicotiana tabacum
MITPKLSLLCIQNPGTQTRSPSGYANESHTTGSENLTQLRLLLSGMGKPRIMHNSREGNEVAHLLAKKTINQSNMDHLVYLAISPSLVETKVLSDKDGESSLKFVVDDACRMSNMDFMKVFDQTVREIKREVNLKVLKVPEIEQKVLDATDDEPWGPHGTALAEIAQATKKFSECQMVMNVLWTRLTETGKNWRYVYKSLAVVEYLVAHGSERAVDEIVEHTYQISSLTSFEYVEPNGKDMGINVRKKAENIVALLNNKEKIEDARNKAAANRDKYFGLSSSGVTFKSSSASLNSSSNFQSGDRYGGFGNKSDGDSFKDSYREKDRYGEDKFDQFKSKKGSSRYGSNVQDTVSSSGSKTSKRVGKPDKATSNPPHSAAVSSSKYEEDFDDFDPRGTSSTKPSTEKSDQVDLFGQNLIGDLLDVPTPVPADNSTVSSHPSEVDLFADANFALAKPQSEISVDLFASQPASSSAAPSTIDFFSAPDPVVQSDIRSPKSDKINATTVDPFAAVPLNTFDSSDPFGTFVSHADPVSVASENANRGGNQEETPSKLDKSSVEAKPAPKKDDFQVRSGIWADSLSRGLIDLNISAPKKVNLADIGIVGGLTDGSDVKEKGPTTFYMGRAMGQGTGLGQSGFTSTSTGGDDFFSSHQNYQFGSFQK

SEQ ID NO: 27 - protein, 284 aa, Nicotiana tabacum
MAMDDNFHRQRLGAHAPPGYFVRLENGRAKDDLYLRKGGRMRKWLCCTCQVEESDPSHENELHKSPKNNFDGYQKGSKASVPAKAEVQKAIPTIEVPALSLDELKEETDNFGSKALIGEGSYGRVYYANLNNGKAVAVKKLDVSSEPETNVDFLSQVSMVSRLKHVNLVDLLGYCVEGNLRVLAYEKGVQGAQPGPTLDWMQRVKIAVDAARGLEYLHEKVQPPIIHRDIRSSNVLLFEDYKAKLLILICQISLLTWLLAFILHEFWEHLVIMHQSNVLHLTLL

SEQ ID NO: 28 - protein, 485 aa, Nicotiana tabacum
MPPPPSTISDDDSSYFHVSTLLPLYRARLRKKLNLRRLRNGTCRAEFANDAPIAVAIGACIFSSLVFPTTYTEDDDGDSVIDSADARFAVMGIISFIPYFNWMSWVFAWLDTGKQRYAVYALVYLAPYLRTNLSLSPEDSWLPIASILLCIFHIQLEVSIKNGDFQALNKFTGTGEELSSVSRKKDDSISEEDMIAGDVVNPDHIDVGFDSIGGLGGIKDTLFQLAILPLRRPELFCHGKLLGPMKGVLLYGPPGTGKTMLAKAIAKESGAVFINVKVSTLMSKWFGDAQKLVAAIFGLAYKLQPAIIFIDEVDSFLGQRRASETEMLTSMKTEFMALWDGFTTDQNARVMVLAATNRPTDLDEAILRRFSQSFEIGKPSLSDRTKIFKVVLKGERIEDNVDFDRLAGLCEGYTGSDILEACKLAAFIPLREYLQDEKKGKQSQAPRPLSQSDLETALAQSKKTKITARKPAVVSFRLDDYEDLD

SEQ ID NO: 29 - protein, 444 aa, Nicotiana tabacum
MSSHDIRRPFKRPAISDQQRRRELSLLRQCQNRRDAQLQARRLASTVLSLQPTQDDDYKSASEEQQLDIEVASVPEVDSFPDETDADFGHPRDAHDIRQATKLRGPEARQWFAKQLMLPEWMIDVPDNLNTDWYVFARPAGKRCFVVSSNGTTISRLRNGIRLHRFPSALPNGARINNSKSAQSYCILDCIFHESDETYYVIDGVCWAGLSLYECTAEFRFFWLNSKLAETGACDAPSTYHRYKFSTLPVYNCDKEGLHTAYVGQVPYVKDGLLFYNKHAHYQTGNTPLTLVWKDENCSQYVIDTDNRGQVPSQQQVVLELLDDSRLATSDDPPVIFGCLLGEFIQKTELQRGDLIKFAIGEGGLVFVDSKLEKADLQYLGKSNRARAFADSYSKVLFQYAARHSPLRIEHLFASISSCVEDGRSNSRCRYGWLKCHARETFFN

SEQ ID NO: 30 - protein, 829 aa, Nicotiana tabacum
MAQPLLKKDDDRDDEAEYSPFMGIEKGAVLQEARVFNDPQLDARRCSQVITKLLYLLNQGETFTKVEATEVFFAVTKLFQSKDLGLRRMVYLMIKELSPSADEVIIVTSSLMKDMNSSTDMYRANAIRVLCRITDGTLLTQIERYLKQAIVDKNPVVASAALVSGIHLLQTNPEIVKRWSNEVQEAVQSRAALVQFHALALLHQIRQNDRLAVSKLVTSLTRGTVRSPLAQCLLIRYTSQVIREAAMSNQTGDRPFYDYLEGCLRHKAEMVIFEAARAITELSGVTSRELTPAITVLQLFLSSSKPVLRFAAVRTLNKVAMTHPMAVTNCNIDMESLISDQNRSIATLAITTLLKTGNESSVDRLMKQITNFMSDIGDEFKIVVVEAIRSLCLKFPLKYRSLMNFLSNILREEGGFEYKKAIVDSIVILIRDIPDAKESGLLHLCEFIEDCEFTYLSTQILHFLGNEGPKTSDPSKYIRYIYNRVILENATVRASAVSTLAKFGALVDSLKPRIFVLLKRCLFDGDDEVRDRATLYLNTLGGDGAVVETDDEVKEFLFGSLGVPLTNLETSLKNYEPSEEAFDIFSVPKEVKSQPLAEKKAPGKKPTGLGAPPVGPTSTVDSYERLLSSIPEFASYGKLFKSSAPVELTEAETEYAVNVVKHIFDSHVVFQYNCTNTIPEQLLENGRHLWLLRKPEGVPAVGKFSNTLRFIVKEVDPTTGEAEDDGVEDEYQLEDLEVVTADYMLKLGVSNFRNAWESLGPDCERGTEVVPSNSRSHTCLLSGVYIGSVKVLVRLSFGLDGAKEVAMKLAVRSEDISVSDAIHEVVASG

SEQ ID NO: 31 - protein, 566 aa, Nicotiana tabacum
MKMRGYSFFTATLILVAVSIFLSSIHTDALPRDTFKSILGEGNLESWKNGVLDSAGMAQAPGPADGHVGTLVLAGNRTRRPDFLSGFHKYRGGWDIANKHYWASVGFTGLAGIILALLWFISFGLALVVHYCCGWKFNIRGREWHFSQNICLGVLIVLTCAAAIGCVLLSVGQDDFHAEALDTLKYVVNQSDYTVQTLRNVTQYLLLAKTVNVAQIFLPSDVKDDIDHLNGDLDSAADKLEDKTNENSGKIRRVFNAVRSALITIAIVMLLISILGLCLSILGHQHAIHIFIISGWLLVAVTFVLYGVFVIINSAISDTCMAMGEWVDNPHAESALSNILPCVDPRTTNRTLFKSKQVTVDLVNIVNGFIDTYANSNPSNHANSNYYNQSGPVMPHLCYPYDSQLQDLPCPADQVSMANSSMVWQNFTCNVSAAAICTSVGRLTPDMYGQLVATVNISYALEHYAPPLLNLQNCDFVRDTFRNITVNHCPPLEHHLRVVNAGLAVISVGVMLSLALWIVYANRPQREEVFAKLSSRIKSSCNGKNISCSNSNIDLSSRGTTPKTGV

SEQ ID NO: 32 - protein, 280 aa, Nicotiana tabacum
MLDASAVAYIQIASIAIADQVARGQIELAKNVRQRLTSPSVAAPPLSTGKQKGGSSSCCKLAASTSSAQMLTSVLSSLVAEEAASLSSGLKSAGFSSSLPFASPEKRLKLDKPMTFSDMNSSEGGNSTYFTSSQQPITSIPLAPSSGLQSSNQIQAPFPPPPPPPPPLPPANSPGSQLGQSAAMMMGMMPYGYSAGSLQPPQIAMGLRPPPPLPQQAQQLHLQTQQPQSQQQPANGGFYRPLVLDSMDRPISRQHQQHPGSKSLWNREHMLHCTLIVKVD

SEQ ID NO: 33 - protein, 225 aa, Nicotiana tabacum
MESDAHFLAKEDGIIAGIALAEMIFAEVDPSLKEMADAAHPAYILETRKTAPGLRLVDKWAVLIGGGKNHRMGLFDMVMIKDNHISAAGGVGKALKSVDQYLEQNKLQIGVEVETRTIEEVREVLDYASQTKTSLTRIMLDNMVVPLSNGDIDVSMLKEAVELINGRFDTEASGNVTLETVHKIGQTGVTYISSGALTHSVKALDISLKIDTELALEVGRRTKRA

SEQ ID NO: 34 - protein, 717 aa, Nicotiana tabacum
MTEVATSNVVHDVLGRRAEDVDQPIIDYIINVLADEDFDFGLDGEGAFEALGELLVDSGCVADFPECRAVCSKLSEKLEKHGLVKPQPTVRSLKMPLRMYDGMDEEEAPKNKKPEPVDGPLLTERDKIKIERRKRKDERLREAEYQAHLKEVEEVKAGMPLVCVNHDGQGDGPTVKDIRMENFNISVAGRDLIVDGSVTLSFGRHYGLIGRNGTGKTTLLRHMAMHAIDGIPKNCQILHVEQEVVGDDTSVLQCILNTDMERTQLLEEEGRLLELQREIDLEGEAGKSDKLNGEIDKNALAKRLEEIYKRLDFIDAYSAESRAATILSGLSFTTEMQKRATKTFSGGWRMRIALARALFIEPDLLLLDEPTNHLDLHAVLWLETYLVKWPKTFIVVSHAREFLNTVVTDIIHLQNQKLSTYKGDYDTFERTRDEQVKNQQKAFEANERTRAHMQTFIDKFRYNAKRASLVQSRIKALERIGRVDEVINDPDYKFEFPSPDDRPGAPIISFSDASFGYPGGPLLFKNLNFGIDLDSRVAMVGPNGIGKSTILKLISGELQPTSGTVFRSAKVRIAVFSQHHVDGLDLSSNPLLYMMRCFPGVPEQKLRGHLGSFGITGNLALQPMYTLSGGQKSRVAFAKITFKKPHILLLDEPSNHLDLDAVEALIQGLVLFQGGVLMVSHDEHLISGSVDQLWAVSEGRVTPFDGTFQDYKKILQS

SEQ ID NO: 35 - protein, 384 aa, Nicotiana tabacum
MGAQISVSKLANLSFVPRIRVPVPNISVPSPSISSGFVSNLSCSAIGISSVLVNFYQSASLAKSANPSTYTYTVPSSPSEVLYRWHLPEPNVVDISGNYDCSSVKSRTVVVLLGWLGAKQKHLKRYAEWYASAGYHVITFTFPMSEILSYQVGGKAEQDIELLVNHLVDWLEEEHGKNLVFHTFSNTGWLTYGVILEKFQKQDPVLMTRIKGCIVDSAPVAAPDPQVWASGFSAAFLKKNSVATKHIMTINNKDADVTIETKTSSDATPAVTEAALLVVLEKFFEVVLSLPAVNRRLSDVLDLLTSQQPSCPQLYIYSSADRVIPAISVESFVEEQRRIGRNVRACNFISTPHVDHFRNDPELYTLQLTQFLEDSVLSSCKQSS

SEQ ID NO: 36 - protein, 547 aa, Nicotiana tabacum
MGGAEDAEPPSKRVKVSSGKPGDLSNGTFPRDPASCSLNDLMARPLVCQGDDEVVGTKGVIKKVEFVRILAEALYSLGYNKTGAHLEEESGIPLQSAVVKLFMQQVLDGKWDESVATLRKIGLVDEKVVQLASFLILEQKFFELLDEKKVMDALKTLSTEIGPLCINTDRVRELSLCILSPLQQVRAVVSGQVVVRAKPRRKLLEELQKLLPPTVIIPEQRLIRLVEQALDLQLDACRFHNSLVGEMSLLTDHQCGRDQIPSQTLQVKLDGLFCMKHQFSGHQKPVSYMSWSPDDHQLLTCGVEEVVRRWDIESGECTHIYEKNGLGLISCGWAPDGKRILCGVTDKSISMWDLEGKELECWKGHRTIRISDLGITSDGQHIVSVCKDNMILLFGWESKAEKVIQEDQTITSFVLSMDSKYLLVSLWNQEIHLWNIEGTVKLISKYKGHKRSRFVVRSCFGGLGQAFVASGSEDSQVYIWHRSSGELIETLAGHSGTVNCVSWNPANPHMLASASDDHTIRIWGMNQVNMKHYDTVSNGVHYCNGGT

SEQ ID NO: 37 - protein, 587 aa, Nicotiana tabacum
MAMVDEPLYPIAVLIDELKNDDIQLRLNSIRRLSTIARALGEERTRKELIPFLSENNDDDDEVLLAMAEELGVFIPYVGGVEHAHVLLPPLETLCTVEETCVRDKAVESLCRIGSQMRESDLVDWFVPLVKRLAAGEWFTARVSACGLFHIAYSSAPEMLKAELRSIYSQLCQDDMPMVRRSAATNLGKFAATVESTYLKSDIMSIFDDLTQDDQDSVRLLAVEGCAALGKLLEPQDCVAHILPVIVNFSQDKSWRVRYMVANQLYELCEAVGPEPTRTDLVPAYVRLLRDNEAEVRIAAAGKVTKFCRILSPELAIQHILPCVKELSSDSSQHVRSALASVIMGMAPVLGKDATIEHLLPIFLSLLKDEFPDVRLNIISKLDQVNQVIGIDLLSQSLLPAIVELAEDRHWRVRLAIIEYIPLLASQLGIGFFDDKLGALCMQWLQDKVYSIRDAAANNLKRLAEEFGPEWAMQHIIPQVLDMTTSPHYLYRMTILRAISLLAPVMGSEITCSKLLPVVITATKDRVPNIKFNVAKVLQSLIPIVDHSVVEKTIRPSLVELAEDPDVDVRFYANQALQSIDNVMMSG

SEQ ID NO: 38 - protein, 621 aa, Nicotiana tabacum
MGFIESCSQTAEMEIRKCSPFLESELLSGNGGLPLTEWRTVPDIWRTSAEKFGDRVAVVDPYHDPPTTMTYKQLYQEIVDFSEGLRVVGLNPNEKIALFADNSCRWLVADQGTMASGAINVVRGSRSSNQELLQLYSHSESVALAIDNPEMYNRISDTFGSHTAVRFAILLWGEKSSLGREAVQGYPVYTYKEIIELGHKSRVDLLDSEDARKQYSFEAINSDDVATIVYTSGTTGNPKGVMLTHKNLLHQILNLGEIVPAVPGDRFLSMLPPWHAYERACEYFIFTHGTEQVYTTVKNLKPHYLISVPLVYETLYSGILKQINSNSAASKLIALLFLRISMTYMEAKRIYEAGVSGGGSLSSHVDKFFEAIGIKIQNGYGLTESSPVISARHLACNVLGSVGHPIRHVEVKIVNAETDEVLPPGSRGIVKARGPLVMKGYYKNPLATKHAIDENGWLNTGDLGWIAPDHSVGRSRKSGGVIVLEGRAKDTIVLSTGENVEPSEIEEAAMGSSLIQQIVVIGQDQRRLGAIIVPNKEEVLLAAKKSAIVDSETTEVSKEKAVGILYEELRKWTSGCSFQVGPILIVDEPFTIDSGLLTPTMKIKRDKIAALYKEQIENLYK

SEQ ID NO: 39 - protein, 869 aa, Nicotiana tabacum
MGGDETKKTTIMVLKVDLQCSSCYKKVKKVLCKFPRCGVIKSIEIKEPPKPKAPEKPKEPVKPKEPEKPKQPEKPKEPEKPKQPEKTTVVVIEKPKEPEKPKAPEKSKEPEKPKEPEKPKEVEKPKPKEPEKPKEAPKPNPVAPPSQPPPPPAPEPIMVQQYPQPPLGYCCGQCYEGHIGGPCYQWYGRPVLPAPCYDNYGYNYGPGPGPGPYGYGRGCYVSRCDQYFSEENATGCSIIKWEPNIVASKAKEVYSAVWVRLLQLPTEFYDRIVLSRIGNSIGRLLRIDACTSSTLRRRYARLCVQVQMDQLVQTTIQIGSHIQQLVYEGEKFLCKACGRLGNTTSTCSHTLLDFQKQQQEEPCPNSTGFIGKERQLKSNDKPSPSPKVTSQKEAQPMDLKIKLKRHLQVSMSIFLMRHQPILTSNKFESLLNDSSITFPEIIESQMELDGQNSNLSPDSKLSFSPRNHSSSLLPPLSPRGQKATCNSNKPHKTAGPSTNPLPCTPLPTLMTPITTENPITDLSLTTCQLAMQLNSPILASLRLHVKNRTMHTKFLLTDFQSLPHENQSPSSPSSSPTHYESTPLLPSNENPSKITLTPPSFLNTVQNEPPTPGYKPSDLHHQCPLTGPPTNAMVGTRTSGPSGFLHSQYPKSRSPVCSTEPTGPSSINLGGYEASNVELHQPPLVDKCSRAHSPTLAPALLHNMQAHWNPPHHFNPLQNMQLFYQLPFFAPQDQNTPPLIHAWQPIPQHYPAPMDFHHTHSHPHHSAPVPGEQEMETQNQTIPPLNITSYTENSNLHEVKILLFQAMEEKKYVRKCPTELYKCSLDIRPPLNAAVGNFAVILSPSLNGLPVPPMGSQFNVTPQPTPIND

SEQ ID NO: 40 - protein, 369 aa, Nicotiana tabacum
MHILYTCPLILFMALLFAYAAATAADLETNGAEKAGADAGILSSNSSVNENLDLINMNRKKDGHLDNDSSNVGDQNKSNDSSAKKGDDREGLKEAEVEKKRIDSGSKRDDRKEETKEAEQQDKAKDISSEKQGEMEKILPDGIQSREEILPTRKESFHGEECDSSYSCTIEEKAVVACLRVPGNESPDLSLLVQNNGKGTVNILIKAPEFVQLEKEKIELQGKENQRMKVSIRNAGNDNNIILKAGDGQCTLDFRGLIDNADKTSQFKYGFLSFAIMCLAAIALVATVLMYFKRRLLVSSGHKYQKLDMDLPVSSGRKTETLSTDGWDNSWDDDWDDEEAPKAPSVPVTPSFSSKSITSRRSSKESWKD

SEQ ID NO: 41 - DNA, 957 bp, Nicotiana tabacum
  source 1..957: mol_type = genomic DNA
atgtgtgtgtactccaattgtaacaaaaaacgtgggagagggagagagagaggagtcgtgattcaagggaagacagcagttggcaataataagactacaaattactatctctactttctgattttcgctccttcactcacatccacagcacatcgcttctccgatccaaaaaaagttgcgaagatgaacgacgcagatgtatcgaagcagatccagcagatggtcagattcatccgccaggaagccgaagaaaaagcctatggatttccgtctccgccgaagaagtattggcctatttcttattctattgatcaattagctttagttagactaattttgagtaataatgtgcgcattgaagaagaattcaacatcgagaagttgcagctagtggaactggagaagaagaagatcaggcaggaatacgagcgtaaggagaaacaagtcgatgttcgcaagaaaattgagtactccatgcaactcaacgcctctcgaatcaaggttcttcaagctcaggatgacttggtcaactccatgaaggaggcagcatcaaaggagcttttaaacgtcagccatcaccagaaccaccatatttataagaagcttctgcaggatcttattgttcagagtttgctcagacttaaagagccttgcgtcctactacgttgtcgggaagatgatgtttccttggtagaaggggtcttggatgcagcaaaagaggagtatgcagaaaaagctcaggttcactcaccggaggtcataattgaccaaatctaccttccatcagctccatcacatcacaatgctcatggctcttcttgctatggaggagtagttttggcttctcgagatgggaaaattgtatgtgaaaatacacttgatgccagattggaagttgtgttccgtaagaaactaccggagattcgcaagtgtctatttggtcaggttgctgcctaa

SEQ ID NO: 42 - DNA, 1980 bp, Nicotiana tabacum
  source 1..1980: mol_type = genomic DNA
atgattactccaaagctatctctcctttgcatacaaaatcctggcactcagacaaggtctccaagtggctatgcaaatgaatctcataccaccggaagtgaaaatttgactcaactgaggttattactgtccggaatggggaagccaaggattatgcataactccagggaaggaaatgaagtagcccacctactggctaagaagacaatcaatcaatctaacatggatcatctcgtctatctggcaatttctccttctcttgttgagaccaaggtgttgtcagacaaagatggagaatcttctctaaaatttgttgtagacgatgcttgtaggatgtccaatatggatttcatgaaggtattcgatcaaacagttcgcgaaataaagagggaggtgaatttgaaagtgttgaaggttcctgagattgagcagaaggtattggacgctacggatgatgaaccttggggcccccatggtactgcattggctgagatagctcaggctacaaaaaaattctctgagtgtcagatggttatgaatgtcctgtggacaagattgactgaaacaggaaagaattggcgttatgtttataagtctttggctgttgttgagtatttggtggctcacggatctgaacgcgctgttgatgagatcgtagaacatacctatcagatatcttctctcacaagtttcgagtatgttgaacccaatgggaaagatatggggatcaatgtgaggaagaaagcagaaaatattgtggcactattgaataacaaggaaaagatcgaagacgctagaaataaagctgctgcaaatcgcgacaagtactttggattgtcatcttctggagtaacatttaaatcgagctctgcctccctaaatagcagcagcaactttcagagtggtgatcgatatggaggttttggaaataaaagtgatggcgattcatttaaggatagttacagggaaaaggatcggtatggtgaagataaatttgaccagtttaaatcaaagaaggggtcttctcgttatggaagcaatgttcaagacactgtttcatctagtggatcaaagacgtcaaagagggtaggtaaacctgataaagctacttctaatcctccacatagtgcagctgtatcatcaagcaaatatgaggaagattttgatgattttgatcctcgagggacttcaagtactaagccttccaccgaaaaatctgaccaagtagatctatttggacaaaatttgattggtgacctcttggatgtaccaacacctgttccagctgataattctactgtctccagtcatccatcagaggttgatttatttgctgatgccaattttgcattggcgaaaccacagtctgagataagtgtagatctgtttgcttctcagcctgcctcttcatctgcagctccttcaaccatagattttttttctgcaccagatcctgttgtacaatccgatatcagatctcctaaatcagacaagataaatgctactacggttgatccgtttgctgcagttccactaaatacctttgatagttctgatccctttggtacatttgtttctcatgctgatcctgtatcagtagccagtgaaaatgctaatcgtggtgggaatcaggaggagactcctagcaaattagataaatcttctgtcgaagctaagcccgcaccaaagaaggatgattttcaagtcaggtctggaatatgggctgattcattgagccgtggactgattgatctgaatatctctgcacccaaaaaggtcaaccttgcagacataggcatcgtgggtggattgaccgatgggtcagatgtgaaagaaaaagggcctactacattttacatgggcagagccatgggtcaaggaaccgggcttggccaatccgggttcacgtccacatcaacgggtggagatgactttttttcaagtcaccagaactatcaatttggcagcttccaaaagtga

SEQ ID NO: 43 - DNA, 855 bp, Nicotiana tabacum
  source 1..855: mol_type = genomic DNA
atggcgatggatgacaatttccataggcaacgactaggggcacatgcacctccgggttactttgtccgcttggagaacggaagggctaaagacgatctttacttgagaaagggaggaaggatgagaaagtggctctgctgcacctgccaagtagaggagtctgacccatcgcatgaaaacgagctccacaaaagccccaagaacaattttgatggatatcagaaagggtcaaaagcatcagttcctgccaaggctgaagtgcaaaaggcaataccaactatagaggtccctgcattgtctttggatgaactgaaagaggaaactgacaattttggatcgaaggcattaattggtgaaggatcttacggaagagtatactatgctaatctaaacaatggcaaagccgtggctgtaaaaaagcttgatgtttcatctgagcctgagactaatgttgacttcctgagccaggtctctatggtttcaagattgaagcatgtaaatctggttgatttgcttggttactgtgtggaagggaaccttcgtgtattagcttatgagaaaggagtacaaggagcacaacctggacctacacttgattggatgcaacgggtaaaaattgctgttgatgctgcaaggggccttgagtatttgcatgagaaggtccagcctccaataatacacagggatatcagatcaagcaatgtccttctctttgaagactacaaagcaaaattgctgattttaatctgtcaaatcagtctcctgacatggctgctcgccttcattctacacgagttttgggaacatttggttatcatgcaccagagtaatgtcttgcaccttactcttctttga

SEQ ID NO: 44 - DNA, 1458 bp, Nicotiana tabacum
  source 1..1458: mol_type = genomic DNA
atgccaccgccgccgtccaccatctccgacgatgattcctcttacttccacgtcagcactcttctacctctatatcgagctcgcctccgcaaaaaacttaatctgagaaggttgagaaatggaacttgtagggcagagtttgcaaacgacgcgccaatcgccgtcgctatcggtgcttgcattttcagctcgttggtttttccgactacttacacggaggatgatgacggggactccgtgattgattctgctgatgcgaggtttgctgttatgggaattatcagcttcattccgtattttaattggatgagttgggttttcgcgtggttggatactgggaagcagcgttacgctgtttatgctcttgtgtatttggctccatatttaagaaccaatctgtctctttctcctgaagacagctggctaccaattgctagcatcctcttgtgcatcttccacattcaactagaagtaagtatcaaaaatggagattttcaggcattgaacaaatttactgggactggagaggaactatcatcagtttctaggaagaaagatgatagcatctctgaagaggatatgattgctggggatgtcgtgaatccagaccacatagatgtggggtttgattcgattggggggcttggtgggattaaggatactttgtttcagctggccattttacctctgcgaaggcctgaattgttttgtcatgggaaattgcttggtccaatgaaaggggttctgttgtatggaccacctgggacagggaagacaatgcttgctaaagccattgctaaagagtctggtgctgtgttcattaatgtgaaggtttctactctcatgagcaagtggtttggtgatgcgcaaaagcttgttgctgctatttttggtttggcctataagctccagcctgctataatatttattgatgaagttgacagctttttgggccagcgtcgtgcaagtgagactgaaatgctgactagcatgaaaactgagttcatggccttatgggatggttttactactgatcagaatgctagagttatggtcctggcagcaaccaatcgcccaactgaccttgatgaggcaatacttaggcgcttttctcagtcatttgagattgggaaaccttcccttagtgatagaacaaagatatttaaggtagtattgaagggtgagagaattgaagataacgttgactttgatcgacttgctggcttgtgtgagggatacactggttcagacattctcgaggcctgcaagctagctgcctttattcctcttagggagtatttgcaagatgagaagaaaggaaagcaatcacaggctccaaggccattgtcacagtctgatctagagacagctttggctcaatcaaagaagaccaagattactgctaggaaacctgctgtagtgagctttcggttggatgattatgaggatttagactga

SEQ ID NO: 45 - DNA, 1335 bp, Nicotiana tabacum
  source 1..1335: mol_type = genomic DNA
atgagctcacacgatatacgccgcccgttcaaacggccggcgatctcagaccaacaaagacgccgagaactttcattgctccggcagtgccaaaaccgccgcgacgctcagttacaagcccgtcgtttagcttccacagtcctctctcttcaacccacgcaagacgatgactacaagtccgcttccgaagagcaacagctggatatagaagttgcttccgtccccgaagttgattcctttccggatgaaaccgacgccgattttggacatcctagggacgcacatgatattcgtcaagctactaagctcagaggacctgaagctcgtcagtggttcgccaagcagcttatgcttcctgaatggatgattgatgttcctgataacttgaacacggattggtatgtatttgctaggccagctggaaagagatgttttgttgtttcttcaaacggaacaacaatcagtagactgcgcaatggaattcgcttgcaccgttttccttctgctctacctaacggtgccagaattaataacagtaaatctgctcaatcatactgtattctcgattgcatatttcacgagtctgatgaaacatattatgtcattgacggtgtatgttgggcgggactttcgttatatgagtgcacggcggaattcagattcttttggttaaacagcaagcttgctgagacgggggcttgtgatgctccctctacttatcatagatataaatttagtacacttcctgtctacaactgtgacaaagaaggactacacacagcttatgtaggacaagttccatatgtcaaggatggattactgttttacaacaagcatgcacattaccaaacaggaaatacaccgttaacattggtttggaaggatgagaactgtagccagtatgtcattgatacagataatagaggacaagttccaagtcaacaacaggtagttttggagctcctagatgatagcagactggctacatctgatgatcctcctgtcatatttggttgcttgcttggggaattcatacaaaagacagaacttcagcgtggagatcttataaagtttgctataggtgaaggcggattagtttttgttgacagtaaactggagaaagctgatctacaatacttgggcaaatccaatcgtgctcgtgcttttgctgatagttactcgaaggtcttgttccagtacgctgctcgacattctcctctgagaattgaacatctttttgcatcaatcagttcatgtgtcgaagatggaagatcaaactcaagatgcagatatggctggttaaagtgccatgcacgggaaacttttttcaactaa

SEQ ID NO: 46 - DNA, 2490 bp, Nicotiana tabacum
  source 1..2490: mol_type = genomic DNA
atggctcagcctctcctgaagaaagacgacgatcgcgatgacgaagcggagtactctccgtttatggggattgagaagggggctgtgcttcaggaagctagggttttcaatgatccgcaattggatgcacgcagatgctcacaggtcattacaaagcttctgtatcttctgaatcagggtgagacgtttacaaaggttgaagctacagaagtgttctttgctgtcacaaaactctttcagtcaaaggatcttggtctaaggagaatggtatacttgatgataaaagagctttctccctcagctgatgaggtaatcatcgttactagctctcttatgaaggacatgaatagcagtacagatatgtatcgtgcaaatgctattcgagtcctctgccgaatcacagatgggactcttctcacacaaattgagagatacttgaaacaagcgattgttgacaaaaaccctgttgttgcaagtgctgcccttgttagtggaatccatttgcttcagacaaacccggagattgtgaaaagatggagcaatgaggtccaagaagctgttcagtcaagggcagctctcgttcaattccatgcactggcgctgctgcaccagataaggcaaaatgaccgtttagctgtgagcaagcttgttaccagtttgacaagaggaactgttcgctcacctctagctcaatgcctcttgattcgttatactagtcaggttataagagaggctgccatgagtaatcaaacaggggataggccattctatgactatctagagggttgcctacgtcacaaagcagaaatggttatttttgaagctgccagggcaatcacagagcttagtggtgtgactagtcgagaattaactcctgcaatcactgttctacagctctttttaagctcttccaagccagttcttaggtttgctgctgttcgaaccttgaataaggtggcaatgacacatcctatggctgtgacaaactgcaacatagatatggagagcttgatttctgatcagaataggagcatagcaactcttgccataacgactcttcttaagaccggcaatgaatcaagtgttgatcgtttgatgaagcagataactaattttatgtccgacattggtgatgagttcaagattgttgtggtggaagccattagatcattgtgtttgaagtttcccctgaagtacagatctttgatgaatttcttaagcaatattttgagggaagaaggaggatttgagtacaaaaaggctattgttgactcaattgtgatcctgatcagagacattccagatgctaaagaaagtgggctgcttcacttgtgtgaatttattgaggactgtgaatttacatacctttctactcagatactacattttcttggaaatgaaggacctaagacatcagaccccagtaagtacatacgatatatatacaatagagttatacttgagaatgcaacagttcgggccagtgcagtgagcaccttagctaagtttggtgccttggttgattcattgaagccccgtatatttgtgctattgaaacgttgcctattcgacggtgatgatgaggttcgcgatagggcaacactgtatttgaatacccttggaggtgatggtgcagttgttgaaactgatgatgaggtgaaagagttcctattcgggtcactcggtgtccctctaaccaatctggagacaagtttaaagaactatgagccatcagaggaggcgtttgatattttttctgttcccaaggaagttaaatctcagcctttggcagagaagaaagcaccgggtaaaaagccaactggtttgggtgctccacctgtcggccccacctctactgttgattcatatgaaagattactgtcctctatcccagaattcgctagctatgggaagcttttcaagtcatcggcgccagtggagctcacagaagctgaaacagagtatgcagttaatgtcgtgaagcacatttttgatagtcatgtagtgttccagtacaactgcaccaataccattcctgagcaattgttggaaaatggcagacatttgtggcttttgagaaaacctgaaggagtccctgctgttgggaaattctcgaacacactaagattcattgttaaagaggttgatccaaccactggtgaggctgaagatgatggtgttgaagatgaataccaactagaagaccttgaggttgtcactgcagattacatgctgaaattgggagtctccaattttaggaatgcatgggagagcttgggaccagattgtgaacgcggcactgaggtagtcccaagcaactcaagatcgcacacatgtttattatctggtgtatacattggcagcgtaaaggtacttgttcggttatcatttggattggatggggcaaaggaggttgcaatgaagctggctgttaggtcagaagatatatctgtaagtgatgcaattcatgaagttgttgcaagcggctag

SEQ ID NO: 47 - DNA, 1701 bp, Nicotiana tabacum
  source 1..1701: mol_type = genomic DNA
atgaaaatgaggggctattcattctttactgctactctcattcttgttgctgtttctatttttcttagctcaatccatactgatgctttaccaagagatactttcaagtctattttaggggagggaaatctggaatcatggaagaatggagtattggactcagcaggtatggcacaagcgccaggtcctgccgatgggcatgttggcacgcttgtgctagcgggaaacaggacgaggagaccggactttctttctggtttccacaaatacagaggtggatgggatattgccaataaacactactgggcttctgttggttttacaggccttgctggtatcatacttgctctgctttggtttatttcatttggcttggctctcgtcgtgcattattgctgcggatggaaattcaatatcagaggcagagaatggcatttttcacagaatatttgcctgggcgtgcttattgtcttgacatgcgctgcagcgattggatgcgtcctactttctgttggacaagatgactttcatgctgaagcattggacactttaaaatatgttgtaaatcagtcagattatactgtgcagacattgagaaatgtaacgcaatacttgttactcgcaaaaactgtaaatgtggcccagattttcctcccttcagatgtaaaagatgatatcgatcacctaaatggcgatctagattctgcagcggataaacttgaggataaaacaaatgaaaactcaggaaagatacgaagggtcttcaatgctgtgcgttcagctttgatcactattgccatcgtcatgctcctcatctctattcttggtctttgcctctctatccttggccatcaacacgcaattcacatatttatcattagtggatggttactggtggcagttacattcgttctctatggagtttttgtcatcataaacagtgcaatttcagatacttgtatggcgatgggagagtgggtggacaatccgcatgctgaaagtgctcttagcaacatccttccatgtgttgacccgagaactacaaaccggacgctgttcaagagcaaacaagtcactgttgatcttgtaaatattgtcaacggatttatcgacacatatgcaaattccaatccatctaatcatgccaattcaaattactataatcagtcaggacccgttatgccacatctctgctatccatatgactcccaattgcaagatcttccgtgccctgctgatcaagtttctatggcaaattcttcaatggtttggcagaactttacttgcaacgtatctgcagctgcaatatgcactagtgtcgggaggctgactcctgacatgtacggacagttggtggcgacggtcaacattagctatgcacttgaacattatgcaccaccgttgcttaatctccagaactgtgatttcgttcgtgatacatttaggaacatcacggtcaaccactgccctccgttggaacaccatcttcgggttgttaatgcaggattagctgtcatatcagtcggagtcatgctaagtctcgcattgtggatagtatatgcaaaccgcccccaaagggaggaagtgtttgcgaagctctcttcgcgaataaagagcagctgtaacggcaagaatattagctgcagtaatagtaatattgatttgtcatcaagaggtacaactccaaagactggagtgtag

SEQ ID NO: 48 - DNA, 843 bp, Nicotiana tabacum
  source 1..843: mol_type = genomic DNA
atgttggacgcgtcagcagtagcatatattcagattgctagcattgccatagcagaccaggttgcacgtggtcagatcgaactagcaaaaaatgtaagacagaggctgacatctcccagtgttgctgctccaccattgagtactggaaagcaaaagggcggcagcagctcttgctgcaaacttgctgcttcaacatcttctgctcaaatgttgacctctgttctttcgtctcttgttgctgaagaagctgcgtcactgagcagtggattgaaatcagctggtttttcttctagcttaccttttgcatctccagagaaacggctcaagttagacaagccaatgactttttctgatatgaacagttccgaagggggtaattccacttacttcacttcatcacagcaaccgattactagcattcctcttgccccttcctcaggcttacaatcgtcaaaccagatacaagctccgtttccaccacctccacctccaccaccacctttacctccagcaaattcccctggaagtcagttaggtcagtctgcagctatgatgatggggatgatgccctatggatatagtgccggcagccttcagccacctcaaattgcaatgggactgaggccacctccaccactaccccagcaagcacaacagctgcatctccagactcagcagccacaatctcaacagcagcctgccaatggtggattttatcgtcctctggtattggattctatggacagacccatcagcagacaacaccagcagcacccaggcagtaaatccctttggaatagggagcacatgttacattgtacattaatagtaaaagtagactag

SEQ ID NO: 49 - DNA, 678 bp, Nicotiana tabacum
  source 1..678: mol_type = genomic DNA
atggaatccgatgctcattttctagcaaaggaagacgggatcatagcaggaattgcacttgctgagatgatattcgcggaagttgatccttcattaaaggaaatggcagatgctgcacaccctgcttacatcttggagactaggaaaactgctcctggattacgtttggtggataaatgggcggtattgatcggtggggggaagaatcacagaatgggcttatttgatatggtaatgataaaagacaatcacatatctgctgctggaggtgtcggcaaagctctaaaatctgtggatcagtatttggagcaaaataaacttcaaataggggttgaggttgaaaccaggacaattgaagaagtacgtgaggttctagactatgcatctcaaacaaagacttcgttgactaggataatgctggacaatatggttgttccattatctaacggagatattgatgtatccatgcttaaggaggctgtagaattgatcaatgggaggtttgatacggaggcttcaggaaatgttacccttgaaacagtacacaagattggacaaactggtgttacctacatttctagtggtgccctgacgcattccgtgaaagcacttgacatttccctgaagatcgatacagagctcgcccttgaagttggaaggcgtacaaaacgagcatga

SEQ ID NO: 50 - DNA, 2154 bp, Nicotiana tabacum
  source 1..2154: mol_type = genomic DNA
atgacggaagtggcgacgagcaacgttgtgcacgacgtcttagggcgacgagctgaagatgtagatcagccgattattgactatataatcaatgttctagccgatgaagatttcgattttggacttgacggtgaaggtgcttttgaagccctcggcgaattactcgtagattctggttgcgttgctgacttccccgaatgtcgtgcggtttgtagcaagttgtctgagaagttagagaagcatggattggttaaacctcaaccaactgtgagaagcttaaaaatgccgctgagaatgtatgatggaatggatgaagaggaagctccaaagaataaaaagccagaaccagttgatggtcctttgctcacagaacgtgacaagattaagatcgaaaggaggaagaggaaagatgaacgcctgagagaggcagaataccaagcacacttgaaagaagtggaagaagtgaaagctggtatgccgttagtgtgtgtgaatcatgatggtcagggtgatggaccaactgttaaggatatccgtatggaaaatttcaatatatctgttgctggtcgtgaccttattgtcgatggttctgttacgctttcttttggaagacactatggccttattggaagaaacggtacggggaaaacaactctcctaagacacatggctatgcacgctattgatggtattcccaagaactgccagatattgcatgttgagcaagaagtggttggtgatgatacctcagttttgcaatgtattcttaacactgatatggagagaacccaacttctggaagaagagggtcgtctgcttgaattacagagagaaattgacctagaaggcgaagctggaaagagtgataagttgaatggggagatcgacaaaaatgccctcgcgaaaaggcttgaagagatatacaaaagacttgatttcattgatgcttactcggctgagtcacgtgcagcaactatactttcgggtttgagcttcactacagaaatgcaaaagagagcaactaaaacattttctggaggatggagaatgagaatagctcttgctcgggcgttgttcattgaacctgatctattgttgcttgatgaacccacgaatcatcttgatctacatgctgtcttatggctggaaacttacctggtgaagtggccgaagacatttatagttgtctctcatgctagagagttcttgaatactgtagtcacagacattatccatctacaaaatcagaaattgagtacctacaaaggagactatgatacattcgaaaggacacgagatgaacaagttaagaatcaacagaaggcgttcgaggcgaatgaacgtacaagggcccacatgcagacctttattgataagttccggtacaatgcaaagcgtgcatctcttgttcaatctagaattaaggcactggaacgaattggtcgtgtggatgaagtcatcaatgatcctgactacaagtttgagttcccttctcctgatgatagacctggtgctcctattataagcttcagtgatgcatcctttggatatcctgggggcccattattgttcaaaaatttgaattttggaatagatctggatagccgagtagcaatggttggtcctaatggtattggaaagtcaacaatacttaagcttatttctggggagcttcaaccaacttcaggaactgttttccgctctgctaaggtccgaattgctgtatttagtcagcatcatgttgatgggctggatctgtcctcaaatcccctcttatacatgatgcgttgctttccaggagtgcctgaacaaaaattacgtggtcatctaggttcatttggtatcactggaaatcttgctcttcagcccatgtacactttgtctggtggccaaaaaagcagagttgcatttgcaaagataaccttcaagaagcctcacatattgcttcttgatgagccatcaaatcacttggatcttgacgctgtggaggctctgatacaaggtcttgtcttgttccaaggaggcgtactgatggtcagtcacgatgaacatttaatatctggtagtgttgatcaactctgggccgtctctgagggcagggtgacgcctttcgacgggacattccaggattacaagaaaattctgcaatcataa

SEQ ID NO: 51 - DNA, 1155 bp, Nicotiana tabacum
  source 1..1155: mol_type = genomic DNA
atgggtgctcagatatccgtatctaagcttgccaatttgtcctttgtccccagaatacgcgttcctgtgcctaacataagtgttccaagtcccagcatcagttctggttttgtttcaaatctttcgtgttctgccataggcatttcttccgtgttggtcaatttttatcaatcagcatcattggctaagtcggcaaatccatcaacatatacttatacagttccttcttcgccttcagaggtgttgtatagatggcatttaccagagccaaatgtcgttgatatatcagggaattatgattgttcatcagtaaagtctaggactgtggtagtactgttgggatggttaggtgcaaaacagaagcatctaaagagatatgcagagtggtatgcctcagcaggatatcatgtcattacatttactttcccaatgtctgagattcttagctatcaagtcgggggaaaggcagagcaggatatagaactgcttgtgaaccatcttgttgattggttggaagaagagcatggaaagaacttggtcttccacactttcagtaacacgggatggttaacttatggtgtcattttggagaagtttcagaaacaagatcctgttttaatgacaaggatcaaaggttgtattgttgattctgctcctgtagctgctcccgatccacaggtatgggcttctggattctctgctgcctttttgaagaagaatagtgttgcaaccaaacacatcatgactataaacaacaaagatgcagatgtgacaatagaaaccaaaacttcttcggatgctacacctgcagtaactgaagcagctttgctagtagtactggagaagttctttgaggtggttttgagccttcccgccgtaaataggagactttctgatgttctagatctattgacatcccagcaaccaagttgcccacaattgtacatatacagcagtgcagacagagtgattcctgcgatttctgttgagtcctttgtagaggagcaacgaagaattggtcgcaacgttagagcttgcaacttcatttctacacctcatgttgatcatttcagaaatgacccagaattatatactttacagcttacccaatttcttgaggactccgttctaagctcttgcaaacagtcttcctga

SEQ ID NO: 52 - DNA, 1644 bp, Nicotiana tabacum
  source 1..1644: mol_type = genomic DNA
atgggaggtgcagaggatgctgaaccaccatccaaacgtgtgaaagtatcctctgggaaaccaggagatctttcaaacggcacatttcctagagatcctgcaagttgctcattgaatgacttgatggctcgccccctggtttgtcaaggggacgatgaggttgttggtacaaaaggggtcatcaagaaagttgaatttgtgcgaattttagctgaggcattatattctcttggttataacaaaactggggcacatctagaagaagagtctgggatacctttgcaatctgctgtggtaaagttatttatgcagcaagtccttgatggtaaatgggatgaaagtgtagccacattacgtaaaatcggtctagtggatgaaaaggttgttcaattggcatcatttctgatattggaacagaagttttttgaactgttggatgaaaaaaaagtcatggatgctttgaagacattgagcactgagattggacctctttgcataaacactgatagagtccgtgagctttctttgtgcattttatcacctttgcagcaggttcgtgctgtggtgtcaggtcaagttgttgtgagagcaaagccacgaagaaagctactagaggaattgcaaaaattgcttcccccaacagttataattcctgaacaaagattgatacgtcttgttgaacaggctcttgacttgcaactagatgcttgtaggtttcacaactctttggtaggtgagatgtctttgctcactgatcatcagtgcggaagggatcaaattccttctcaaactttgcaggtgaaattggatggtttgttctgcatgaagcaccagttttctggtcaccagaaacctgtctcctatatgtcatggagtcctgatgaccatcagcttctcacttgtggagtagaggaagttgtcagacggtgggatattgaatcaggtgaatgtacacatatttatgagaaaaatggtcttggtctgatctcatgtggatgggctcctgatggcaaaaggatattatgcggtgttacggacaagagcattagcatgtgggatctggaagggaaagagttggagtgttggaaaggccatcgaactattagaatatctgacttggggataactagtgatgggcagcatatagtctctgtttgcaaagataatatgatattactatttggatgggaatcaaaagcagagaaagtaattcaggaggatcaaacaataacttcatttgtattgtccatggacagtaagtatttattggttagtctttggaatcaagaaatccatctgtggaatatagagggaactgtaaagctcatatccaaatataaagggcataaacgttcacgctttgttgtaaggtcttgctttggcggactgggtcaagcatttgttgccagtggaagtgaggactcacaggtttatatatggcatagaagctcaggagaactcattgagacattggctggacattctggtacagtaaactgtgttagctggaacccagcaaatcctcatatgttggcatctgcaagtgatgatcatactattcgcatatggggcatgaatcaagtaaacatgaaacactatgacacagttagtaatggcgtgcattactgcaatggcggaacttag

SEQ ID NO: 53 - DNA, 1764 bp, Nicotiana tabacum
  source 1..1764: mol_type = genomic DNA
atggcaatggtagatgagccattgtaccccatagccgtgttaatagatgaacttaagaacgatgatatacaattacggttgaattcaattaggaggttatcgactattgcacgtgcccttggtgaggaaagaactcgaaaggaattgatcccttttttgagtgaaaacaatgatgatgatgatgaggtgttattggcaatggctgaagagcttggtgtgtttatcccttatgttggaggtgtagagcatgctcatgttttgctcccgccgttggagacgctttgtactgttgaggagacctgtgtgagggataaagctgttgaatcgttgtgtaggattggatctcagatgagggagagtgatttggttgattggttcgtccctcttgtgaagaggctggcagctggtgaatggttcacagctagagtttctgcctgtggactctttcatattgcttactcaagtgccccagagatgttgaaggcagaacttcggtctatttacagtcaattgtgtcaagacgacatgcctatggtgcgaagatcagctgccacaaacttggggaagtttgctgctactgttgaatctacttacctcaagagtgacatcatgtcaatatttgatgatcttacacaggatgatcaggattctgtacgcttattagctgttgagggctgtgctgcacttggcaagctgttggagccccaggattgtgttgcacacatcctgcctgtcattgtcaacttctctcaggacaagtcttggcgcgtccgctacatggttgctaaccagttgtacgaactatgtgaagctgtagggcctgagcccactaggacggatttggtgcctgcctatgtccgtttgcttcgagataatgaagctgaagttcgcatagctgctgcagggaaagtcaccaaattctgtcggattcttagtcccgagcttgctattcagcatattcttccctgtgtgaaggaattatcatcagactcttcacagcatgtcagatctgctttggcttctgttataatggggatggctcctgttttgggaaaggatgcaaccattgagcatcttcttccaatatttctttcccttctgaaggacgagtttcctgatgtgcgcctgaacatcattagcaagcttgatcaagtcaatcaggtgattggaattgatttattatcccaatctttgttgccagctattgttgagctagcagaggacaggcattggcgagtccgtcttgcaataatagaatacatacctctattggcaagtcaattgggcataggattttttgatgataagcttggtgccctttgtatgcaatggttacaggacaaggtttattcaatcagagatgctgctgctaataacctaaagcgtcttgcagaagaatttggtccagagtgggcaatgcagcatataattcctcaggtcttggatatgactaccagtccacattatttgtatagaatgacaattcttagagcaatttcattgcttgcacctgtaatgggctctgaaataacttgttctaaattgctgcctgtggttattactgcaacaaaggatagagtgcccaacattaaatttaatgtggcaaaggtgttgcaatcccttatacctattgttgaccactcggtggtggagaaaaccattcgccctagtttagtagagctagctgaagaccctgatgttgatgttcgcttttatgccaatcaagcacttcagtcaattgataacgtcatgatgtcaggctag

SEQ ID NO: 54 - DNA, 1866 bp, Nicotiana tabacum
  source 1..1866: mol_type = genomic DNA
atgggattcatcgaatcatgttcccagactgcagaaatggaaatcaggaagtgctcgccttttctggaaagtgaattgttgtccggtaatggtgggttgcccttgacagagtggagaactgttcccgacatttggcggacttcggcagagaagtttggtgaccgtgtagcagttgtggacccatatcatgatcctcctacaaccatgacttataaacagctttatcaggagattgtggatttctctgaaggtttgagagttgttgggctaaacccaaatgagaagattgcgctttttgctgataattcatgtcgatggcttgttgcagatcaaggtacgatggcgagtggggctatcaacgttgtgaggggttcaaggtcatcaaatcaagagctattgcaattatacagccactctgaaagtgtcgctcttgctattgacaatcctgagatgtacaaccggatttcagacacctttggttcccacacagctgtacgatttgctattttactttggggcgagaaatcaagccttggaagagaagccgtgcagggatatcctgtatatacttataaggagattatagaattgggtcacaagagtcgtgtggatctgcttgattctgaagatgccaggaaacaatattcatttgaggcaatcaactctgatgatgtggctacaattgtctataccagtggaaccaccggtaatccaaaaggtgtcatgcttacgcataaaaatctgcttcaccagattttgaatctgggggagattgtacctgctgtacctggggacagatttctaagcatgcttccgccttggcatgcatatgagcgtgcttgtgaatatttcatattcacacatggaacagagcaagtgtacacaactgtgaaaaatttgaagccacattacttgataagtgttcctttagtttatgagacattatacagtggaattctaaagcagatcaattcaaactctgctgctagtaaactcattgccctattatttttaaggatcagtatgacttacatggaggcaaaaaggatttacgaggctggcgtaagtggaggtggtagtctttcttcacatgttgacaagttctttgaggcaattggcataaagattcagaatggatatggtctgactgagtcatctcccgtgatttctgcccgtcatcttgcgtgtaatgtacttggctcagttgggcatcccattcggcatgtagaagtaaaaattgtaaatgctgaaacagatgaggtccttcctcctggctcaaggggcattgtcaaagccagagggccactagtaatgaagggctactataagaatccgttggcaacaaaacacgctattgatgagaatggatggctgaacactggtgatcttggttggattgcgcctgatcattctgtagggcgaagtcgtaaaagtgggggtgtaatagtccttgaaggccgtgcaaaggataccatagtcctttcaactggcgagaatgttgaaccatcagagattgaagaagctgcaatgggaagtagtctgatccagcagattgttgtcattggccaggatcaacgacgtcttggagctataattgtaccaaataaggaggaggttctgttagcagctaaaaaatcagctattgtggattctgaaaccactgaagttagcaaggaaaaagcagttggcatattatatgaggagttaagaaaatggacttcaggttgctcatttcaagttggacctatccttattgtcgatgaacctttcacgattgatagtggcttactaacaccaaccatgaaaatcaagagagacaaaattgcagctctatacaaagagcaaattgagaacttgtacaaatga

SEQ ID NO: 55 - DNA, 2610 bp, Nicotiana tabacum
  source 1..2610: mol_type = genomic DNA
atgggtggtgacgaaactaaaaagaccactataatggtgctcaaggttgatcttcagtgctccagctgctataagaaggtcaaaaaagttctctgtaaattccctcgatgtggagtaattaagagcattgaaatcaaagaacctccaaagcccaaagctcctgaaaagcccaaggagcctgtgaaaccaaaagaacccgagaagcccaaacaaccggagaagcccaaagagcccgaaaagcctaaacaacctgagaagactacggttgtcgtcattgaaaagcccaaggagcccgagaagcctaaggcaccggaaaagtcaaaagagcccgagaagcccaaagaaccagaaaagccgaaagaagttgaaaagcccaagcccaaagagcccgaaaagcccaaagaagctcctaaacctaatccagtggctccaccatcacaaccaccaccaccgccagcaccagagccaataatggttcaacaataccctcagccaccactaggatattgttgtggacaatgctacgagggtcatatcgggggcccatgttatcaatggtacggaagacctgtccttccggccccatgttacgataactatgggtataactatgggcctgggcctgggcctgggccgtatggctacggaaggggttgttatgtgagtagatgtgaccaatactttagtgaagaaaatgccacaggatgctcaattataaagtgggagccaaatattgtggctagcaaagctaaagaagtctactccgccgtttgggttcgcctcctccaattacccacagaattctatgatagaattgtccttagtagaattggaaactcaattggccgactactacgcatagatgcttgtacaagttcaacgcttagaaggaggtacgcgcgactgtgtgtgcaagtgcaaatggaccaactagtccaaacaacaatccagattggctcccatatacaacaactggtatatgaaggagaaaaatttctttgtaaggcctgtgggcgactgggaaacacgacatcaacatgctcccacactctacttgacttccaaaagcaacaacaagaagagccatgtcctaactcgactggcttcataggcaaggaaaggcaattgaagagcaatgacaaaccgtctccttccccaaaggtaacaagccaaaaagaggcacaaccaatggatctcaaaataaaactcaagcgacacctccaggtatcaatgtcaatatttttgatgcggcatcagcccatccttacatctaataaattcgaatcacttcttaatgatagcagtattacattcccggaaataattgaatcccaaatggaattggatgggcaaaactctaacctctccccagactctaaactgtcgttctctccaagaaaccattcctcttccctgctccctcctctctctccacgtggccaaaaggctacatgcaactccaataaaccacacaaaacagcgggcccatccactaatcctctaccatgcacgccacttcccacactaatgacacctattaccactgaaaaccctataactgacttgtcacttaccacctgccaattggccatgcaactcaactctccaatactggctagtctaaggttgcacgttaaaaatcgaaccatgcacacaaaatttctactaacagattttcaatcattacctcacgaaaaccaatccccctcatccccttcatcttctcctacacactatgaatctactccactcctcccatcaaacgaaaatccctctaaaataactctcacaccaccatcgttcctcaatactgtccagaatgaaccacccactcctggatacaaaccttccgatctacatcaccaatgccctctcactggaccaccaaccaacgccatggttggaacaaggacttcaggtccaagtggctttcttcactctcaatatccaaaatcacgaagtcctgtttgttctacagaacccacaggtcctagctcaattaatctcggagggtatgaggctagcaatgtcgaactacatcaaccacctttagttgacaaatgttctagagcccatagccccaccctagcaccagcccttcttcacaacatgcaagcccactggaatccaccacatcacttcaatcccctacaaaacatgcaacttttttaccaacttcccttttttgctccacaggatcaaaacaccccacccttgatccatgcatggcaacccatccctcaacactacccagctccgatggattttcaccacacacattcccatccccatcacagtgcacctgtaccaggagagcaagaaatggaaacccaaaatcaaaccatcccaccactaaatataacatcgtacacagaaaattcaaacctacacgaggtcaaaatcttgttattccaagcgatggaagaaaagaagtatgtccgcaaatgtcccacagagctatacaaatgctccctcgacataaggcccccactcaatgcagcagtaggcaatttcgccgtaatactcagtccatcattgaatggtcttcctgttccaccaatggggagccagttcaatgtcaccccccagccaacacccatcaatgactag

SEQ ID NO: 56 - DNA, 1110 bp, Nicotiana tabacum
  source 1..1110: mol_type = genomic DNA
atgcatatattgtatacttgtccgctgattctgtttatggctttactctttgcttatgctgctgctactgctgctgatttagagaccaatggagctgaaaaggcgggtgcggatgcaggaattttgagtagtaatagcagtgtgaatgagaatttagacctgataaatatgaatagaaaaaaggatggtcatttggataacgatagttcgaatgttggggatcaaaacaagtccaatgatagtagtgcgaaaaagggtgacgacagagaagggttgaaagaagctgaggtggaaaagaaaagaattgatagtggttctaagagagatgacaggaaggaggagacgaaagaagctgaacagcaagacaaagcaaaagatattagttccgagaagcagggtgagatggaaaagattctgccggatggaattcagtcgagagaggagattttgcctacaagaaaggagagtttccatggtgaagaatgcgattcatcttatagttgcacgatagaggaaaaagcagtggttgcatgtcttagagttccgggcaatgaatctccagacctttcacttttggttcaaaacaatggaaaaggaactgtcaatattttgattaaggctcctgagtttgtacaactggagaaagagaagattgaactgcaaggaaaggaaaatcagaggatgaaggtttctataaggaatgcaggaaatgacaacaatatcattctaaaggccggggatggccaatgcactcttgatttcaggggtctgattgacaatgctgataaaacatctcaattcaagtatggtttcctatcttttgcaataatgtgtttggctgctattgcattagtggccacagtcttgatgtactttaaacggaggcttctagtaagtagcggccacaagtatcaaaagttggacatggatttaccagtttccagtggcagaaagacggagacactctcaactgatggatgggacaatagctgggatgacgattgggatgatgaggaggcgcccaaagcaccatccgtgccagtcactccttccttctcgtctaaaagcattacctcacgacggtctagtaaggaaagctggaaagactag

SEQ ID NO: 57 - DNA, 314 bp, Nicotiana tabacum
  source 1..314: mol_type = genomic DNA
cttttctcaagaaacatatttattaaataatttctttttcgttacattcatttttataatctattcctttatgttctcttagagaaaagtatactattctaaaatacatttatcccaaaaaatagaaaaatgaatgtagtattcatktactcaagcgtgttatattttgtcaataaaatgtctcccaaaatattcaaatcttgttcaataccacatttcgtgataatccttcctgtatattaattaaaatctattttaccccagaattgtactaaaaaagtcagctaggtgtactacctatttcgcattcatgc

SEQ ID NO: 58 - DNA, 160 bp, Nicotiana tabacum
  source 1..160: mol_type = genomic DNA
agcccgatttgtcgtgaggatttgatatgtctccctccaaaggtagcagctagtttrggaaatatcggtcctcttgtgatctgcaccaaagtgagcaacaatattgctttattagayccgtttactctgaggcattgtttcttggatgctgatcagtact

SEQ ID NO: 59 - DNA, 322 bp, Nicotiana tabacum
  source 1..322: mol_type = genomic DNA
aaaagtgcgatctaaaacgatgcagtaaccaaaccgaaggggcctgttgcccatatccaccaagtttcaggacgtgtttaacatttagctaacaaattctctcatgagatttaactcgagttagagcttcaacctgcatgggcataagtgtacaatacatcrggtatgttactggtgaccaagagtttaagactgaaaaattggagggcgacaataagagtactataccttttgcaggcaccacaaccacaaatttcagacatgtcatgaaagatgcgtagcctcctactgtggcagaggaaagcatgcatggttaatttgg

SEQ ID NO: 60 - DNA, 71 bp, Nicotiana tabacum
  source 1..71: mol_type = genomic DNA
cataaattttgtccaacatggaagtatataggtcamgttgtagttgatttcaaacacttgttagaaaagta

SEQ ID NO: 61 - DNA, 71 bp, Nicotiana tabacum
  source 1..71: mol_type = genomic DNA
tttcccgctggggatttcagttgtattcctctctgytgtttctttacttcgggctcgacttgtggtcttcc

SEQ ID NO: 62 - DNA, 71 bp, Nicotiana tabacum
  source 1..71: mol_type = genomic DNA
acagtaaattttctcactcttgattctgcatcaaayacacttcttgtgctgttaagattcttcatagttgt

SEQ ID NO: 63 - DNA, 71 bp, Nicotiana tabacum
  source 1..71: mol_type = genomic DNA
cgcctgtgcataatgaggcgcatatggtgggtatgstttattttagaaaggaatatgagagttcatctgga

SEQ ID NO: 64 - DNA, 71 bp, Nicotiana tabacum
  source 1..71: mol_type = genomic DNA
ttgtaattattatgatagaattagtattgctttatwtattgtatttttatccctttaaaagtcaactgctg

SEQ ID NO: 65 - DNA, 11801 bp, synthetic construct
  misc_feature 1..11801: note = Description of Artificial Sequence: Synthetic polynucleotide
  source 1..11801: mol_type = other DNA
tgagcgtcgcaaaggcgctcggtcttgccttgctcgtcggtgatgtacttcaccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgccagaaggccgccagagaggccgagcgcggccgtgaggcttggacgctagggcagggcatgaaaaagcccgtagcgggctgctacgggcgtctgacgcggtggaaagggggaggggatgttgtctacatggctctgctgtagtgagtgggttgcgctccggcagcggtcctgatcaatcgtcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggcatcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgcttgacactttatcactgataaacataatatgtccaccaacttatcagtgataaagaatccgcgcgttcaatcggaccagcggaggctggtccggaggccagacgtgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccagatctggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgaagcttccagaaggtaattatccaagatgtagcatcaagaatccaatgtttacgggaaaaactatggaagtattatgtgagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttgtggatcctgcaggctagcgtgcactctagactcgacgaactgacgagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactatgataaattatcgcgcgcggtgtcatctatgttactagatcgggaattcctcgagcaactatttttatgtatgcaagagtcagcatatgtataattgattcagaatcgttttgacgagttcggatgtagtagtagccattatttaatgtacatactaatcgtgaatagtgaatatgatgaaacattgtatcttattgtataaatatccataaacacatcatgaaagacactttctttcacggtctgaattaattatgatacaattctaatagaaaacgaattaaattacgttgaattgtatgaaatctaattgaacaagccaaccacgacgacgactaacgttgcctggattgactcggtttaagttaaccactaaaaaaacggagctgtcatgtaacacgcggatcgagcaggtcacagtcatgaagccatcaaagcaaaagaactaatccaagggctgagatgattaattagtttaaaaattagttaacacgagggaaaaggctgtctgacagccaggtcacgttatctttacctgtggtcgaaatgattcgtgtctgtcgattttaattatttttttgaaaggccgaaaataaagttgtaagagataaacccgcctatataaattcatatattttcctctccgctttgaattgtctcgttgtcctcctcactttcatcagccgttttgaatctccggcgacttgacagagaagaacaaggaagaagactaagagagaaagtaagagataatccaggagattcattctccgttttgaatcttcctcaatctcatcttcttccgctctttctttccaaggtaataggaactttctggatctactttatttgctggatctcgatcttgttttctcaatttccttgagatctggaattcgtttaatttggatctgtgaacctccactaaatcttttggttttactagaatcgatctaagttgaccgatcagttagctcgattatagctaccagaatttggcttgaccttgatggagagatccatgttcatgttacctgggaaatgatttgtatatgtgaattgaaatctgaactgttgaagttagattgaatctgaacactgtcaatgttagattgaatctgaacactgtttaaggttagatgaagtttgtgtatagattcttcgaaactttaggatttgtagtgtcgtacgttgaacagaaagctatttctgattcaatcagggtttatttgactgtattgaactctttttgtgtgtttgcagctcataaaaggtaccaaacaatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttttgagcgggactctggcgatcgccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggactagtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatatcaactcccctatccattgctcaccgaatggtacaggtcggggacccgaagttccgactgtcggcctgatgcatccccggctgatcgaccccagatctggggctgagaaagcccagtaaggaaacaactgtaggttcgagtcgcgagatcccccggaaccaaaggaagtaggttaaacccgctccgatcaggccgagccacgccaggccgagaacattggttcctgtaggcatcgggattggcggatcaaacactaaagctactggaacgagcagaagtcctccggccgccagttgccaggcggtaaaggtgagcagaggcacgggaggttgccacttgcgggtcagcacggttccgaacgccatggaaaccgcccccgccaggcccgctgcgacgccgacaggatctagcgctgcgtttggtgtcaacaccaacagcgccacgcccgcagttccgcaaatagcccccaggaccgccatcaatcgtatcgggctacctagcagagcggcagagatgaacacgaccatcagcggctgcacagcgcctaccgtcgccgcgaccccgcccggcaggcggtagaccgaaataaacaacaagctccagaatagcgaaatattaagtgcgccgaggatgaagatgcgcatccaccagattcccgttggaatctgtcggacgatcatcacgagcaataaacccgccggcaacgcccgcagcagcataccggcgacccctcggcctcgctgttcgggctccacgaaaacgccggacagatgcgccttgtgagcgtccttggggccgtcctcctgtttgaagaccgacagcccaatgatctcgccgtcgatgtaggcgccgaatgccacggcatctcgcaaccgttcagcgaacgcctccatgggctttttctcctcgtgctcgtaaacggacccgaacatctctggagctttcttcagggccgacaatcggatctcgcggaaatcctgcacgtcggccgctccaagccgtcgaatctgagccttaatcacaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccacatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccacaattatgggtgatgctgccaacttactgatttagtgtatgatggtgtttttgaggtgctccagtggcttctgtgtctatcagctgtccctcctgttcagctactgacggggtggtgcgtaacggcaaaagcaccgccggacatcagcgctatctctgctctcactgccgtaaaacatggcaactgcagttcacttacaccgcttctcaacccggtacgcaccagaaaatcattgatatggccatgaatggcgttggatgccgggcaacagcccgcattatgggcgttggcctcaacacgattttacgtcacttaaaaaactcaggccgcagtcggtaacctcgcgcatacagccgggcagtgacgtcatcgtctgcgcggaaatggacgaacagtggggctatgtcggggctaaatcgcgccagcgctggctgttttacgcgtatgacagtctccggaagacggttgttgcgcacgtattcggtgaacgcactatggcgacgctggggcgtcttatgagcctgctgtcaccctttgacgtggtgatatggatgacggatggctggccgctgtatgaatcccgcctgaagggaaagctgcacgtaatcagcaagcgatatacgcagcgaattgagcggcataacctgaatctgaggcagcacctggcacggctgggacggaagtcgctgtcgttctcaaaatcggtggagctgcatgacaaagtcatcgggcattatctgaacataaaacactatcaataagttggagtcattacccaattatgatagaatttacaagctataaggttattgtcctgggtttcaagcattagtccatgcaagtttttatgctttgcccattctatagatatattgataagcgcgctgcctatgccttgccccctgaaatccttacatacggcgatatcttctatataaaagatatattatcttatcagtattgtcaatatattcaaggcaatctgcctcctcatcctcttcatcctcttcgtcttggtagctttttaaatatggcgcttcatagagtaattctgtaaaggtccaattctcgttttcatacctcggtataatcttacctatcacctcaaatggttcgctgggtttatcgcacccccgaacacgagcacggcacccgcgaccactatgccaagaatgcccaaggtaaaaattgccggccccgccatgaagtccgtgaatgccccgacggccgaagtgaagggcaggccgccacccaggccgccgccctcactgcccggcacctggtcgctgaatgtcgatgccagcacctgcggcacgtcaatgcttccgggcgtcgcgctcgggctgatcgcccatcccgttactgccccgatcccggcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccgg

(19) United States
(12) Patent Application Publication
(10) Pub. No.: US 2024/0099257 A1
(43) Pub. Date: Mar. 28, 2024

(54) METHODS AND COMPOSITIONS RELATED TO IMPROVED NITROGEN UTILIZATION EFFICIENCY IN TOBACCO

(71) Applicant: Altria Client Services LLC, Richmond, VA (US)

(72) Inventors: Jesse Frederick, Richmond, VA (US); Chengalrayan Kudithipudi, Midlothian, VA (US); Dongmei Xu, Glen Allen, VA (US)

(73) Assignee: Altria Client Services LLC, Richmond, VA (US)

(21) Appl. No.: 18/163,499

(22) Filed: Feb. 2, 2023

Related U.S. Application Data
(63) Continuation of application No. 17/116,816, filed on Dec. 9, 2020, now Pat. No. 11,602,118.
Application No. 17/116,816 is a continuation of application No. 16/119,366, filed on Aug. 31, 2018, now Pat. No. 10,888,064.
(60) Provisional application No. 62/553,501, filed on Sep. 1, 2017.

(51) Int. Cl. A01H 6/82; A01H 5/10; A01H 5/12; C07K 14/415; C12N 9/10; C12N 9/88; C12N 9/90; C12N 15/82

(52) U.S. Cl. CPC A01H 6/823; A01H 5/10; A01H 5/12; C07K 14/415; C12N 9/1085; C12N 9/88; C12N 9/90; C12N 15/8227; C12N 15/8237; C12N 15/8261; C12Y 205/01054; C12Y 402/01033; C12Y 501/03015

(57) ABSTRACT

The present disclosure provides metabolic signatures and genetic markers for tracking enhanced nitrogen utilization efficiency phenotypes in tobacco plants and for introgressing enhanced nitrogen utilization efficiency phenotypes into tobacco plants. The disclosure also provides tobacco plants comprising enhanced nitrogen utilization efficiency and methods to the creation of tobacco plants comprising enhanced nitrogen utilization efficiency. The disclosure also provides recombinant polynucleotides and polypeptides for enhancing nitrogen utilization efficiency in modified tobacco plants and tobacco plants comprising the provided recombinant polynucleotides and polypeptides.

### CROSS-REFERENCE TO RELATED APPLICATIONS

This application is a continuation application of U.S. patent application Ser. No. 17/116,816, filed Dec. 9, 2020, which is a continuation of Ser. No. 16/119,366, filed Aug. 31, 2018, now U.S. patent Ser. No. 10/888,064, issued Jan. 12, 2021, which claims the benefit of U.S. Provisional Application No. 62/553,501, filed Sep. 1, 2017, all of which are incorporated by reference in their entireties herein.

### INCORPORATION OF SEQUENCE LISTING

A sequence listing contained in the file named “P34523US03_SL.TXT” which is 188,493 bytes (measured in MS-Windows®) and created on Feb. 2, 2023, is filed electronically herewith and incorporated by reference in its entirety.

### FIELD

The present disclosure provides compositions and methods useful for making and identifying tobacco plants comprising improved nitrogen utilization efficiency via breeding, transgenic approaches, and cisgenic approaches.

### BACKGROUND

Fertilizer is a major cost for tobacco growers, and increased fertilization has been tied to higher levels of alkaloids and tobacco-specific nitrosamines (TSNAs) in plant tissues. Different tobacco varieties require different levels of nitrogen fertilizer input to achieve the maximum yield for each variety. For example, Maryland tobacco varieties typically require approximately 25% less nitrogen input to achieve maximum yield as compared to Burley tobacco varieties.

Improving Nitrogen Utilization Efficiency (NUE) in tobacco would increase tobacco harvestable yield per unit of input nitrogen fertilizer. Nitrogen utilization efficiency improvement also allows decreases in on-farm input costs, decreased use and dependence on the non-renewable energy sources required for nitrogen fertilizer production, and reduces the environmental impact of nitrogen fertilizer manufacturing and agricultural use.

Methods and compositions for improving the nitrogen utilization efficiency of tobacco are provided herein.

### SUMMARY

In one aspect, the present disclosure provides for, and includes, a method of determining the NUE of a tobacco line comprising obtaining at least one metabolite from a tobacco plant of a tobacco line, determining the amount of the obtained metabolites, and determining the NUE of the tobacco line based on the amount of the metabolites identified.

In one aspect, the present specification provides for, and includes, a method of determining the NUE of a tobacco line using a metabolite signature comprising isolating a metabolite signature from a tobacco plant of a tobacco line, determining the amount of each metabolite comprising a metabolite signature, and determining the NUE of a tobacco line by comparing the metabolite signature to a control metabolite signature from a control tobacco line comprising a known NUE.

In one aspect, the current specification provides for, and includes, a method of breeding a tobacco line comprising a metabolite signature associated with enhanced NUE comprising determining the metabolite signature of a first tobacco plant from a first tobacco line, where a first tobacco plant comprises enhanced NUE as compared to a control tobacco plant lacking the metabolite signature, crossing the first plant with a second plant of a second tobacco line, and obtaining at least one progeny seed from the crossing, where a progeny plant grown from at least one progeny seed comprises the metabolite signature, and where the progeny plant comprises enhanced NUE as compared to a control plant lacking the metabolite signature.

In one aspect, the present specification provides for, and includes, a method of selecting a tobacco plant comprising obtaining a population of tobacco plants, isolating at least one metabolite associated with enhanced NUE from at least one tobacco plant from the population of tobacco plants, and selecting at least one tobacco plant that comprises a higher amount of at least one metabolite as compared to a control tobacco plant. In a further aspect of this method, a selected tobacco plant comprises an enhanced NUE as compared to a control tobacco plant.

In one aspect, the present specification provides for, and includes, a method of selecting a tobacco plant comprising obtaining a population of tobacco plants, isolating at least one metabolite associated with enhanced NUE from at least one tobacco plant from the population of tobacco plants, and selecting at least one tobacco plant that comprises a lower amount of at least one metabolite as compared to a control tobacco plant.

In one aspect, the present specification provides for, and includes, a method of screening a tobacco plant for a first metabolite signature associated with enhanced NUE comprising isolating a first metabolite signature associated with enhanced NUE from a tobacco plant, determining the amount of at least one metabolite that comprises that first metabolite signature, comparing the first metabolite signature to a second metabolite signature of a control tobacco plant comprising a known NUE, and determining if the first metabolite signature is associated with enhanced NUE.

In one aspect, the present specification provides for, and includes, a modified tobacco seed, or tobacco plant grown therefrom, comprising a cisgenic polynucleotide comprising a heterologous promoter operably linked to a coding region, where the modified tobacco plant comprises enhanced nitrogen utilization efficiency as compared to an unmodified control tobacco plant lacking the cisgenic polynucleotide when grown under the same conditions.

In one aspect, the present specification provides for, and includes, a recombinant DNA construct comprising a heterologous promoter operably linked to a polynucleotide encoding a polypeptide at least 70% identical or similar to a polypeptide selected from the group consisting of SEQ ID NOs:1 to 8.

In one aspect, the present specification provides for, and includes, cured tobacco material, or a tobacco product comprising the cured tobacco material, where the cured tobacco material is made from a tobacco plant comprising a cisgenic polynucleotide comprising a heterologous promoter operably linked to a coding region, where the modified tobacco plant comprises enhanced nitrogen utilization efficiency as compared to an unmodified control tobacco plant lacking the cisgenic polynucleotide when grown under the same conditions.

In one aspect, the present specification provides for, and includes, a greenhouse, growth chamber, or field comprising the modified tobacco seed or plant disclosed herein.

In one aspect, the present specification provides for, and includes, a modified tobacco seed, or tobacco plant grown therefrom, comprising at least one mutation in an endogenous locus encoding a polypeptide selected from the group consisting of SEQ ID NOs:25 to 40, and where a modified tobacco seed or tobacco plant comprises enhanced nitrogen utilization efficiency as compared to an unmodified control tobacco plant lacking at least one mutation when grown under the same conditions.

In one aspect, the present specification provides for, and includes, a recombinant DNA construct comprising a heterologous promoter operably linked to a guide RNA comprising at least 18 contiguous nucleotides identical or complementary to a polynucleotide encoding a polypeptide selected from the group consisting of SEQ ID NOs:25 to 40.

In one aspect, the present specification provides for, and includes, cured tobacco material, or a tobacco product comprising the cured tobacco material, where the cured tobacco material is made from a tobacco plant comprising at least one mutation in an endogenous locus encoding a polypeptide selected from the group consisting of SEQ ID NOs:25 to 40, and where the modified tobacco seed or tobacco plant comprises enhanced NUE as compared to an unmodified control tobacco plant lacking the at least one mutation when grown under the same conditions.

In one aspect, the present specification provides for, and includes, a modified tobacco seed, or tobacco plant grown therefrom, comprising a cisgenic polynucleotide comprising a heterologous promoter operably linked to a polynucleotide encoding a small RNA (sRNA) at least 85% identical or complementary to a polynucleotide selected from the group consisting of SEQ ID NOs:41 to 56, and where the modified tobacco seed or tobacco plant comprises enhanced NUE as compared to an unmodified control tobacco plant lacking the cisgenic polynucleotide when grown under the same conditions.

In one aspect, the present specification provides for, and includes, a recombinant DNA construct comprising a heterologous promoter operably linked to a polynucleotide encoding a small RNA (sRNA) at least 85% identical or complementary to a polynucleotide selected from the group consisting of SEQ ID NOs:41 to 56.

In one aspect, the present specification provides for, and includes, cured tobacco material, or a tobacco product comprising the cured tobacco material, where the cured tobacco material is made from a tobacco plant comprising a cisgenic polynucleotide comprising a heterologous promoter operably linked to a polynucleotide encoding a sRNA at least 85% identical or complementary to a polynucleotide selected from the group consisting of SEQ ID NOs:41 to 56, and where the modified tobacco seed or tobacco plant comprises enhanced NUE as compared to an unmodified control tobacco plant lacking the cisgenic polynucleotide when grown under the same conditions.

In one aspect, the present specification provides for, and includes, a method of enhancing the NUE of a tobacco plant comprising introducing a cisgenic nucleic acid molecule into a tobacco cell, and regenerating a modified tobacco plant from that tobacco cell where the modified tobacco plant comprises enhanced NUE as compared to a tobacco plant lacking the cisgenic nucleic acid molecule.

In one aspect, the present specification provides for, and includes, a method of enhancing the NUE of a tobacco plant comprising introducing a modification to a nucleic acid molecule encoding a gene having a sequence selected from the group consisting of SEQ ID NOs:41 to 56 in a tobacco cell and regenerating a modified tobacco plant from the tobacco cell, where the modified tobacco plant comprises enhanced NUE as compared to a tobacco plant lacking the modification.

In one aspect, the present specification provides for, and includes, a method of enhancing the NUE of a tobacco plant comprising introducing a nucleic acid encoding a small RNA (sRNA) homologous to at least 18 contiguous nucleic acids of a nucleic acid molecule encoding a gene having a sequence selected from the group consisting of SEQ ID NOs:41 to 56 in a tobacco cell, and regenerating a modified tobacco plant from the tobacco cell, where the modified tobacco plant comprises enhanced NUE as compared to a tobacco plant lacking the sRNA.

In one aspect, the present specification provides for, and includes, a method comprising providing a first population of tobacco plants comprising enhanced NUE, genotyping a first population of tobacco plants for the presence of a molecular marker within 20 cM of an enhanced NUE locus; and selecting one or more tobacco plants genotyped and found to comprise the molecular marker.

In one aspect, the present specification provides for, and includes, a method comprising providing a first population of tobacco plants, genotyping the first population of tobacco plants for the presence of an enhanced NUE allele of a locus encoded by a sequence selected from the group consisting of SEQ ID NOs:9 to 16; and selecting one or more genotyped tobacco plants that comprise an enhanced NUE allele.

In one aspect, the present specification provides for, and includes, a method of introgressing an enhanced NUE trait into a tobacco variety comprising crossing a first tobacco variety comprising an enhanced nitrogen utilization efficiency trait with a second tobacco variety lacking the enhanced nitrogen utilization efficiency trait, obtaining progeny seed from the cross, genotyping at least one progeny seed for a molecular marker linked to an enhanced nitrogen utilization efficiency trait, where the molecular marker is within 20 cM of a locus having a sequence selected from the group consisting of SEQ ID NOs:9 to 16; and selecting a progeny seed comprising an enhanced nitrogen utilization efficiency trait.

In one aspect, the present specification provides for, and includes, a method of selecting a tobacco plant with an enhanced NUE trait comprising isolating nucleic acids from a collection of tobacco germplasm, assaying the isolated nucleic acids for one or more markers located within 20 cM of a locus selected from the group consisting of SEQ ID NOs:9 to 16, and selecting a tobacco plant comprising an enhanced NUE trait.

In one aspect, the present specification provides for, and includes, a method of selecting a tobacco plant with an enhanced NUE trait comprising isolating nucleic acids from a collection of tobacco germplasm, assaying the isolated nucleic acids for one or more markers located within 20 cM of a marker selected from the group consisting of SEQ ID NOs:57 to 64, and selecting a tobacco plant comprising an enhanced NUE trait.

### BRIEF DESCRIPTION OF THE SEQUENCES

SEQ ID NOs: 1 to 8 are amino acid sequences of genes positively correlated with enhanced NUE in root tissue, leaf tissue, or both.

SEQ ID NOs: 9 to 16 are nucleotide sequences of genes positively correlated with enhanced NUE in root tissue, leaf tissue, or both.

SEQ ID NOs: 17 to 19 are nucleotide sequences of promoter regions for genes with leaf-preferred expression.

SEQ ID NOs: 20 to 24 are nucleotide sequences of promoter regions for genes with root-preferred expression.

SEQ ID NOs: 25 to 40 are amino acid sequences of genes negatively correlated with enhanced NUE in root tissue, leaf tissue, or both.

SEQ ID NOs: 41 to 56 are nucleotide sequences of genes negatively correlated with enhanced NUE in root tissue, leaf tissue, or both.

SEQ ID NOs:57 to 64 are nucleotide sequences of SNP markers comprising polymorphisms associated with enhanced NUE.

SEQ ID NO: 65 is the backbone sequence for expression vector p45-2-7.

### BRIEF DESCRIPTION OF THE DRAWINGS

FIG. 1 depicts four gene clusters associated with NUE in the tobacco genome. Genes differentially expressed between low and normal nitrogen conditions in plants with an NUE metabolite fingerprint are indicated (correlated genes). The total number of differentially expressed genes (DEG), regardless of NUE metabolic fingerprinting, is also indicated.

FIG. 2 depicts a 2 megabase region of tobacco chromosome 11 that is covered by superscaffold 1. Superscaffold1 is a contig of scaffolds A and B. Of the 79 expressed genes located in this region, 56 genes are differentially expressed.

FIG. 3 depicts the allelic constitution for 23 Burley and 6 Maryland varieties at a genetic locus correlated with NUE on tobacco chromosome 11. Lines 1, 2, and 3 are Burley lines that contain a favorable Maryland allele at SEQ ID NO:58, and line 4 is a standard Burley line that contains an unfavorable Burley allele at SEQ ID NO:58.

FIG. 4 depicts chlorophyll loss, growth, and yield for Lines 1 to 4 as described in FIG. 3 and a MD609 control (C). Lines 1, 2, and 3 are Burley lines that contain a favorable Maryland allele at SEQ ID NO:58 and line 4 is a standard Burley line that contains an unfavorable Burley allele at SEQ ID NO:58.

FIG. 5 depicts yield in grams fresh weight per plant of greenhouse grown T₁ plants overexpressing genes positively correlated with increased yield under nitrogen stress. The mean and standard deviation based on 9 plants per sample is displayed.

FIG. 6 depicts yield in pounds per acre after harvest for two independent field grown F₄ lines, NUE-2 and NUE-3. The test lines are generated from crosses of MD609 to Burley TN90 as described. The mean and standard deviation is provided in comparison to control TN90 Burley tobacco.

### DETAILED DESCRIPTION

Unless defined otherwise, technical and scientific terms used herein have the same meaning as commonly understood by one of ordinary skill in the art. One skilled in the art will recognize many methods can be used in the practice of the present disclosure. Indeed, the present disclosure is in no way limited to the methods and materials described. For purposes of the present disclosure, the following terms are defined below.

Unless defined otherwise, all technical and scientific terms used have the same meaning as commonly understood by one of ordinary skill in the art to which this disclosure belongs. Where a term is provided in the singular, the inventors also contemplate aspects of the disclosure described by the plural of that term. Where there are discrepancies in terms and definitions used in references that are incorporated by reference, the terms used in this application shall have the definitions given herein. Other technical terms used have their ordinary meaning in the art in which they are used, as exemplified by various art-specific dictionaries, for example, “The American Heritage® Science Dictionary” (Editors of the American Heritage Dictionaries, 2011, Houghton Mifflin Harcourt, Boston and New York), the “McGraw-Hill Dictionary of Scientific and Technical Terms” (6th edition, 2002, McGraw-Hill, New York), or the “Oxford Dictionary of Biology” (6th edition, 2008, Oxford University Press, Oxford and New York). The inventors do not intend to be limited to a mechanism or mode of action. Reference thereto is provided for illustrative purposes only.

The practice of this disclosure includes, unless otherwise indicated, conventional techniques of biochemistry, chemistry, molecular biology, microbiology, cell biology, genomics, biotechnology, metabolomics, plant breeding, and genetics, which are within the skill of the art. See, for example, Green and Sambrook, Molecular Cloning: A Laboratory Manual, 4th edition (2012); Current Protocols In Molecular Biology (F. M. Ausubel, et al. eds., (1987)); Plant Breeding Methodology (N. F. Jensen, Wiley-Interscience (1988)); the series Methods In Enzymology (Academic Press, Inc.): PCR 2: A Practical Approach (M. J. MacPherson, B. D. Hames and G. R. Taylor eds. (1995)); Harlow and Lane, eds. (1988) Antibodies, A Laboratory Manual; Animal Cell Culture (R. I. Freshney, ed. (1987)); Recombinant Protein Purification: Principles And Methods, 18-1142-75, GE Healthcare Life Sciences; C. N. Stewart, A. Touraev, V. Citovsky, T. Tzfira eds. (2011) Plant Transformation Technologies (Wiley-Blackwell); and R. H. Smith (2013) Plant Tissue Culture: Techniques and Experiments (Academic Press, Inc.).

Any references cited herein, including, e.g., all patents, published patent applications, and non-patent publications, are incorporated by reference in their entirety.

As used herein, the singular form “a,” “an,” and “the” include plural references unless the context clearly dictates otherwise. For example, the term “a compound” or “at least one compound” may include a plurality of compounds, including mixtures thereof.

As used herein, the term “sequence identity” or “identity” in the context of two polynucleotide or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” An alignment of two or more sequences may be performed using any suitable computer program. For example, a widely used and accepted computer program for performing sequence alignments is CLUSTALW v1.6 (Thompson, et al. (1994) Nucl. Acids Res., 22: 4673-4680).

As used herein, the term “complementary” in reference to a nucleic acid molecule refers to pairing of nucleotide bases such that adenine is complementary to thymine or uracil, and guanine is complementary to cytosine. Two complementary nucleic acid molecules are capable of hybridizing with each other. As an example, the two strands of double stranded DNA are complementary to each other.

A specific polynucleotide of at least three nucleotides in length may be referred to as an “oligonucleotide”. Nucleic acid molecules provided herein include deoxyribonucleic acids (DNA) and ribonucleic acids (RNA) and functional analogues thereof, such as complementary DNA (cDNA). Nucleic acid molecules provided herein can be single stranded or double stranded. Nucleic acid molecules comprise the nucleotide bases adenine (A), guanine (G), thymine (T), cytosine (C). Uracil (U) replaces thymine in RNA molecules. The symbol “R” can be used to represent a purine (e.g., A or G) nucleotide base. The symbol “Y” can be used to represent a pyrimidine (e.g., a C or T) nucleotide base. The symbol “W” can be used to represent an A or a T nucleotide base. The symbol S can be used to represent a G or a C nucleotide base. The symbol “M” can be used to represent an A or a C nucleotide base. The symbol The symbol “K” can be used to represent a G or a T nucleotide base. The symbol “B” can be used to represent a G, C, or T nucleotide base. The symbol “H” can be used to represent an A, C, or T nucleotide base. The symbol “D” can be used to represent an A, G, or T nucleotide base. The symbol “V” can be used to represent an A, G, or C nucleotide base. The symbol “N” can be used to represent any nucleotide base (e.g., A, G, C, T, or U).

The use of the term “polynucleotide” is not intended to limit the present disclosure to polynucleotides comprising DNA. Those of ordinary skill in the art will recognize that polynucleotides and nucleic acid molecules can comprise ribonucleotides and combinations of ribonucleotides and deoxyribonucleotides. Such deoxyribonucleotides and ribonucleotides include both naturally occurring molecules and synthetic analogues. The polynucleotides of the present disclosure also encompass all forms of sequences including, but not limited to, single-stranded forms, double-stranded forms, hairpins, stem-and-loop structures, and the like.

As used herein, the term “polypeptide” refers to a chain of at least two covalently linked amino acids. Polypeptides can be encoded by polynucleotides provided herein.

Nucleic acid molecules, polypeptides, or proteins provided herein can be isolated or substantially purified. An “isolated” or “purified” nucleic acid molecule, polypeptide, protein, or biologically active portion thereof, is substantially or essentially free from components that normally accompany or interact with the polynucleotide or protein as found in its naturally occurring environment. For example, an isolated or purified polynucleotide or protein is substantially free of other cellular material, or culture medium when produced by recombinant techniques, or substantially free of chemical precursors or other chemicals when chemically synthesized. In one aspect, an isolated polynucleotide provided herein can contain less than 10000 nucleotides, less than 5000 nucleotides, less than 4000 nucleotides, less than 3000 nucleotides, less than 2000 nucleotides, less than 1000 nucleotides, less than 500 nucleotides, or less than 100 nucleotides of nucleic acid sequence that naturally flank the polynucleotide in genomic DNA of the cell from which the polynucleotide is derived. In one aspect, an isolated polynucleotide provided herein can contain 100 to 10000 nucleotides, 500 to 10000 nucleotides, 1000 to 10000 nucleotides, 2000 to 10000 nucleotides, 3000 to 10000 nucleotides, 4000 to 10000 nucleotides, 1 to 500 nucleotides, 1 to 1000 nucleotides, 1 to 2000 nucleotides, 1 to 3000 nucleotides, 1 to 4000 nucleotides, 1 to 5000 nucleotides, 1 to 10000 nucleotides, 100 to 500 nucleotides, 100 to 1000 nucleotides, 100 to 2000 nucleotides, 100 to 3000 nucleotides, or 100 to 4000 nucleotides of nucleic acid sequence that naturally flank the polynucleotide in genomic DNA of the cell from which the polynucleotide is derived. In another aspect, an isolated polypeptide provided herein is substantially free of cellular material in preparations having less than 30%, less than 20%, less than 10%, less than 5%, or less than 1% (by dry weight) of chemical precursors or non-protein-of-interest chemicals. Fragments of the disclosed polynucleotides and polypeptides encoded thereby are also encompassed by the present invention. Fragments of a polynucleotide may encode polypeptide fragments that retain the biological activity of the native polypeptide. Alternatively, fragments of a polynucleotide that are useful as hybridization probes or PCR primers using methods known in the art generally do not encode fragment polypeptides retaining biological activity. Fragments of a polynucleotide provided herein can range from at least 20 nucleotides, at least 50 nucleotides, at least 70 nucleotides, at least 100 nucleotides, at least 150 nucleotides, at least 200 nucleotides, at least 250 nucleotides, at least 300 nucleotides, and up to the full-length polynucleotide encoding the polypeptides of the invention, depending on the desired outcome.

Nucleic acids can be isolated using techniques routine in the art. For example, nucleic acids can be isolated using any method including, without limitation, recombinant nucleic acid technology, and/or the polymerase chain reaction (PCR). General PCR techniques are described, for example in PCR Primer: A Laboratory Manual, Dieffenbach & Dveksler, Eds., Cold Spring Harbor Laboratory Press, 1995. Recombinant nucleic acid techniques include, for example, restriction enzyme digestion and ligation, which can be used to isolate a nucleic acid. Isolated nucleic acids also can be chemically synthesized, either as a single nucleic acid molecule or as a series of oligonucleotides. Polypeptides can be purified from natural sources (e.g., a biological sample) by known methods such as DEAE ion exchange, gel filtration, and hydroxyapatite chromatography. A polypeptide also can be purified, for example, by expressing a nucleic acid in an expression vector. In addition, a purified polypeptide can be obtained by chemical synthesis. The extent of purity of a polypeptide can be measured using any appropriate method, e.g., column chromatography, polyacrylamide gel electrophoresis, or HPLC analysis.

In one aspect, this disclosure provides methods of detecting recombinant nucleic acids and polypeptides in plant cells. Without being limiting, nucleic acids also can be detected using hybridization. Hybridization between nucleic acids is discussed in detail in Sambrook et al. (1989, Molecular Cloning: A Laboratory Manual, 2nd Ed., Cold Spring Harbor Laboratory Press, Cold Spring Harbor, NY).

Polypeptides can be detected using antibodies. Techniques for detecting polypeptides using antibodies include enzyme linked immunosorbent assays (ELISAs), Western blots, immunoprecipitations and immunofluorescence. An antibody provided herein can be a polyclonal antibody or a monoclonal antibody. An antibody having specific binding affinity for a polypeptide provided herein can be generated using methods well known in the art. An antibody provided herein can be attached to a solid support such as a microtiter plate using methods known in the art.

Detection (e.g., of an amplification product, of a hybridization complex, of a polypeptide) can be accomplished using detectable labels. The term “label” is intended to encompass the use of direct labels as well as indirect labels. Detectable labels include enzymes, prosthetic groups, fluorescent materials, luminescent materials, bioluminescent materials, and radioactive materials.

As used herein, the phrase “associated with” or “linked to” refers to a recognizable and/or assayable relationship between two entities. For example, the phrase “associated with enhanced NUE” refers to a trait, locus, gene, allele, marker, phenotype, etc., or the expression thereof, the presence or absence of which can influence an extent, degree, and/or rate at which a plant or a part of interest thereof that has an enhanced NUE trait. As such, a marker is “associated with” a trait when it is linked to it and when the presence of the marker is an indicator of whether and/or to what extent the desired trait or trait form will occur in a plant/germplasm comprising the marker. Similarly, a marker is “associated with” an allele when it is linked to it and when the presence of the marker is an indicator of whether the allele is present in a plant/germplasm comprising the marker. For example, “a marker associated with enhanced NUE allele” refers to a marker whose presence or absence can be used to predict whether and to what extent a plant will display enhanced NUE phenotype.

As used herein, a “centimorgan” (cM) is a unit of measure of recombination frequency and genetic distance between two loci. One cM is equal to a 1% chance that a marker at one genetic locus will be separated from a marker at a second locus due to crossing over in a single generation.

As used herein, “closely linked” means that the marker or locus is within about 20 cM, 15 cM, 10 cM, 5 cM, 4 cM, 3 cM, 2 cM, 1 cM, 0.5 cM, or less than 0.5 cM of another marker or locus. For example, 20 cM means that recombination occurs between the marker and the locus with a frequency of equal to or less than about 20%.

As used herein, “plant” refers to a whole plant. A cell or tissue culture derived from a plant can comprise any plant components or plant organs (e.g., leaves, stems, roots, etc.), plant tissues, seeds, plant cells, and/or progeny of the same. A progeny plant can be from any filial generation, e.g., F₁, F₂, F₃, F₄, F₅, F₆, F₇, etc. A plant cell is a biological cell of a plant, taken from a plant or derived through culture from a cell taken from a plant.

As used herein, a tobacco plant can be from any plant from the Nicotiana tabacum genus including, but not limited to Nicotiana tabacum tabacum; Nicotiana tabacum amplexicaulis PI 271989; Nicotiana tabacum benthamiana PI 555478; Nicotiana tabacum bigelovii PI 555485; Nicotiana tabacum debneyi; Nicotiana tabacum excelsior PI 224063; Nicotiana tabacum glutinosa PI 555507; Nicotiana tabacum goodspeedii PI 241012; Nicotiana tabacum gossei PI 230953; Nicotiana tabacum hesperis PI 271991; Nicotiana tabacum knightiana PI 555527; Nicotiana tabacum maritima PI 555535; Nicotiana tabacum megalosiphon PI 555536; Nicotiana tabacum nudicaulis PI 555540; Nicotiana tabacum paniculata PI 555545; Nicotiana tabacum plumbaginfolia PI 555548; Nicotiana tabacum repanda PI 555552; Nicotiana tabacum rustica; Nicotiana tabacum suaveolens PI 230960; Nicotiana tabacum sylvestris PI 555569; Nicotiana tabacum tomentosa PI 266379; Nicotiana tabacum tomentosiformis; and Nicotiana tabacum trigonophylla PI 555572.

In one aspect, a plant component provided herein includes, but is not limited to, a leaf, a stem, a root, a seed, a flower, pollen, an anther, an ovule, a pedicel, a fruit, a meristem, a cotyledon, a hypocotyl, a pod, an embryo, endosperm, an explant, a callus, a tissue culture, a shoot, a cell, and a protoplast. In further aspects, this disclosure provides tobacco plant cells, tissues, and organs that are not reproductive material and do not mediate the natural reproduction of the plant. In another aspect, this disclosure also provides tobacco plant cells, tissues, and organs that are reproductive material and mediate the natural reproduction of the plant. In another aspect, this disclosure provides tobacco plant cells, tissues, and organs that cannot maintain themselves via photosynthesis. In another aspect, this disclosure provides somatic tobacco plant cells. Somatic cells, contrary to germline cells, do not mediate plant reproduction.

Provided cells, tissues and organs can be from seed, fruit, leaf, cotyledon, hypocotyl, meristem, embryos, endosperm, root, shoot, stem, pod, flower, inflorescence, stalk, pedicel, style, stigma, receptacle, petal, sepal, pollen, anther, filament, ovary, ovule, pericarp, phloem, and vascular tissue. In another aspect, this disclosure provides a tobacco plant chloroplast. In a further aspect, this disclosure provides an epidermal cell, a stomata cell, a leaf hair (trichome), a root hair, or a storage root. In another aspect, this disclosure provides a tobacco protoplast.

Skilled artisans understand that tobacco plants naturally reproduce via seeds, not via asexual reproduction or vegetative propagation. In one aspect, this disclosure provides tobacco endosperm. In another aspect, this disclosure provides a tobacco endosperm cell. In a further aspect, this disclosure provides a male or female sterile tobacco plant, which cannot reproduce without human intervention.

In one aspect, this disclosure provides methods and compositions related to modified tobacco plants, seeds, plant components, plant cells, and products made from modified tobacco plants, seeds, plant parts, and plant cells. In one aspect, a modified seed provided herein gives rise to a modified plant provided herein. In one aspect, a modified plant, seed, plant component, plant cell, or plant genome provided herein comprises a recombinant DNA construct provided herein. In another aspect, cured tobacco material or tobacco products provided herein comprise modified tobacco plants, plant components, plant cells, or plant genomes provided herein.

As used herein, “modified” refers to plants, seeds, plant components, plant cells, and plant genomes that have been subjected to mutagenesis, genome editing, genetic transformation, or a combination thereof.

As used herein, “cisgenesis” or “cisgenic” refers to genetic modification of a plant, plant cell, or plant genome in which all components (e.g., promoter, donor nucleic acid, selection gene) have only plant origins (e.g., no non-plant origin components are used). In one aspect, a modified plant, plant cell, or plant genome provided herein is cisgenic. Cisgenic plants, plant cells, and plant genomes provided herein can lead to ready-to-use tobacco lines. In another aspect, a modified tobacco plant provided herein comprises no non-tobacco genetic material or sequences.

As used herein, a “functional fragment” or “functional fragment thereof” refers to a nucleotide or amino acid sequence of any size that retains the function of the full length sequence to which it refers. In an aspect, a functional fragment can be at least 5, at least 10, at least 25, at least 50, at least 75, at least 100, at least 150, at least 200, at least 250, at least 300, at least 400, at least 500, at least 600, at least 700, at least 800, at least 900, at least 1000, at least 2000, at least 3000, at least 4000, at least 5000, or more than 5000 nucleotides in length. In an aspect, a functional fragment can be at least 5, at least 10, at least 25, at least 50, at least 75, at least 100, at least 150, at least 200, at least 250, at least 300, at least 400, at least 500, at least 600, at least 700, at least 800, at least 900, at least 1000, at least 2000, or more than 2000 amino acids in length. In an aspect, a functional fragment can be between 5 and 5000 nucleotides, between 10 and 4000 nucleotides, between 25 and 3000 nucleotides, between 50 and 2000 nucleotides, between 75 and 1000 nucleotides, between 100 and 900 nucleotides, between 150 and 800 nucleotides, between 200 and 700 nucleotides, between 250 and 600 nucleotides, or between 300 and 500 nucleotides in length. In an aspect, a functional fragment can be between 5 and 2000 amino acids, between 10 and 1000 amino acids, between 25 and 900 amino acids, between 50 and 800 amino acids, between 50 and 800 amino acids, between 75 and 700 amino acids, between 100 and 600 amino acids, between 150 and 500 amino acids, between 200 and 400 amino acids, or between 250 and 300 amino acids in length. In a further aspect, the polynucleotides described herein are envisioned in their entirety and as any functional fragments thereof. In a further aspect, the polypeptides described herein are envisioned in their entirety and as any functional fragments thereof. In a further aspect, the polynucleotides having the sequence of SEQ ID NOs: 9 to 24 and 41 to 56 are envisioned in their entirety and as any functional fragments thereof. In a further aspect, the polypeptides having the sequence of SEQ ID NOs: 1 to 8 and 25 to 40 are envisioned in their entirety and as any functional fragments thereof.

As used herein, the term “nitrogen utilization efficiency” (NUE) refers to the ability of a plant to absorb, assimilate and/or use nitrogen (e.g., from soil, water and/or nitrogen fertilizer). NUE genes affect yield and have utility for improving the use of nitrogen in crop plants. Enhanced nitrogen utilization efficiency can result from improved uptake and assimilation of nitrogen fertilizer and/or the subsequent remobilization and reutilization of accumulated nitrogen reserves, as well as increased tolerance of plants to stress situations such as low nitrogen environments. NUE genes can be used to alter the genetic composition of a plant, rendering it more productive with current fertilizer application standards or maintaining its productive rates with significantly reduced fertilizer or reduced nitrogen availability.

NUE has been defined in various ways, but yield per unit of nitrogen available in the soil integrates all key parameters for evaluating fitness of crop cultivars and it is a common measure of NUE. See, for example, Ladha et al. 2005. Advances in Agronomy, 87:85-156, which is incorporated herein in its entirety. This indicator is sometimes referred to as “agricultural NUE.” As another measure of NUE, the ratio of the plant product (e.g., tobacco leaf tissue) to above-ground nitrogen in the plant can be determined (sometimes referred to as “physiological NUE).” Enhanced NUE is related to three key components: 1) yield is not significantly different when grown on 25% normal nitrogen content compared to a plant grown at 100% normal nitrogen content); 2) the rate of chlorophyll loss is reduced compared to plants without enhanced NUE; and 3) cured leaf quality is not significantly different when grown on 25% normal nitrogen content compared to a plant grown at 100% normal nitrogen content. In a preferred aspect, a plant with enhanced NUE is capable of generating similar yields and leaf quality when grown under 25% of the Burley fertilization rate as compared to a Burley plant grown under 100% of the normal Burley fertilization rate.

At least five approaches and indices of NUE are used in the art and are discussed below.

(1) Partial factor productivity (PFP) from applied nitrogen (N) is a measure of how much yield is produced for each unit of nitrogen applied:

PFP_{N}=kilograms of yield/kilograms of N applied

PFP_{N}=Y_{+N}/FN
Where Y_{+N} is the yield (kilograms/hectare; kg/ha) and FN is the amount of fertilizer applied (kg/ha).

(2) Agronomic efficiency (AE) of applied nitrogen (N) is a measure of how much additional yield is produced for each unit of nitrogen applied:

AE_{N}=kilograms of yield increase/kilograms of N applied

AE_{N}=(Y_{+N}−Y_{0N})/FN
Where Y_{+N} is the yield in a treatment with N application (kg/ha); Y_{0N} is the yield in a control treatment without N application (kg/ha); and FN is the amount of N fertilizer applied (kg/ha).

(3) Recovery efficiency (RE) of applied nitrogen (N) is a measure of how much of the nitrogen that was applied was recovered and taken up by the crop.

RE_{N}=kilograms of N taken up/kilograms of N applied

RE_{N}=(UN_{+N}−UN_{0N})/FN
Where UN_{+N} is the total plant N uptake measured in aboveground biomass at physiological maturity (kg/ha) in plots that received applied N at the rate of FN (kg/ha); and UN_{0N} is the total N uptake of a control plot without the addition of N.

(4) Physiological efficiency (PE) of applied nitrogen (N) is a measure of how much additional yield is produced for each additional unit of nitrogen uptake.

PE_{N}=kilograms of yield increase/kilograms of fertilizer N taken up

PE_{N}=(Y_{+N}−Y_{0N})/(UN_{+N}−UN_{0N})
Where Y_{+N} is the yield (kg/ha) in a treatment with N application; Y_{0N} is the yield (kg/ha) in a control treatment without N application; UN_{+N} is the total N uptake (kg/ha) in the treatment that receives fertilizer N application; and UN_{0N} is the total N uptake (kg/ha) in the treatment without fertilizer N application.

(5) Internal efficiency (IE) of nitrogen (N) addresses how much yield is produced per unit N taken up from both fertilizer and indigenous (e.g., soil) nutrient sources:

IEN=kilograms of yield/kilograms of N taken up

IEN=Y/UN
Where Y is the yield (kg/ha); and UN is the total N uptake (kg/ha).

Nitrogen can be in any form, including organic and/or inorganic forms. Without being limiting, forms of nitrogen include nitrate (e.g, ammonium nitrate, calcium nitrate, potassium nitrate), nitrite, ammonia, aqua ammonia, anhydrous ammonia, ammonium sulfate, diammonium phosphate, a low-pressure nitrogen solution, a pressureless nitrogen solution, urea, and urea-ammonium nitrate (UAN). In an aspect, nitrogen is in a form that is immediately available to a plant (e.g., ammonia and/or nitrate) and/or can be readily converted to a form that is available to a plant (e.g., urea).

In an aspect, a modified tobacco plant comprising enhanced NUE provided herein comprises increased nitrogen uptake as compared to a control tobacco plant. In another aspect, a modified tobacco plant comprising enhanced NUE provided herein comprises increased nitrogen assimilation as compared to a control tobacco plant. In a further aspect, a modified tobacco plant comprising enhanced NUE provided herein comprises increased yield as compared to a control tobacco plant. In still another aspect, a modified tobacco plant comprising enhanced NUE provided herein comprises increased yield under low nitrogen conditions as compared to a control tobacco plant. In a preferred aspect, low nitrogen conditions as used in the field are approximately 25% nitrogen compared to levels typically used by those skilled in the art. In another aspect, low nitrogen conditions as used in the field can be between approximately 5% and 50% nitrogen compared to levels typically used by those skilled in the art. In a greenhouse setting, low nitrogen conditions are approximately 25 parts per million (ppm) and normal nitrogen conditions are approximately 100 ppm. In another aspect, low nitrogen conditions as used in a greenhouse can be between 5 ppm and 50 ppm.

In an aspect, a modified tobacco plant comprising enhanced NUE provided herein comprises a yield increase of at least 5%, at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 200%, at least 300%, at least 400%, or at least 500% as compared to a control tobacco plant grown under similar growth conditions. In an aspect, a modified tobacco plant comprising enhanced NUE provided herein comprises a yield increase of between 5% and 100%, between 10% and 100%, between 20% and 100%, between 30% and 100%, between 40% and 100%, between 50% and 100%, between 60% and 100%, between 70% and 100%, between 80% and 100%, between 90% and 100%, between 10% and 200%, between 10% and 300%, between 10% and 400%, between 10% and 500%, or between 5% and 500% as compared to a control tobacco plant grown under similar growth conditions.

In an aspect, a population of modified tobacco plants comprising enhanced NUE provided herein comprises a yield increase of at least 0.25 kg/ha, at least 0.5 kg/ha, at least 0.75 kg/ha, at least 1 kg/ha, at least 2 kg/ha, at least 3 kg/ha, at least 4 kg/ha, at least 5 kg/ha, at least 6 kg/ha, at least 7 kg/ha, at least 8 kg/ha, at least 9 kg/ha, at least 10 kg/ha, at least 15 kg/ha, at least 20 kg/ha, at least 25 kg/ha, at least 30 kg/ha, at least 35 kg/ha, at least 40 kg/ha, at least 45 kg/ha, at least 50 kg/ha, at least 75 kg/ha, at least 100 kg/ha, at least 200 kg/ha, at least 300 kg/ha, at least 400 kg/ha, or at least 500 kg/ha as compared to a population of control tobacco plants grown under similar growth conditions. In another aspect, a population of modified tobacco plant comprising enhanced NUE provided herein comprises a yield increase of between 0.25 kg/ha and 100 kg/ha, between 0.5 kg/ha and 100 kg/ha, between 0.75 kg/ha and 100 kg/ha, between 1 kg/ha and 100 kg/ha, between 2 kg/ha and 100 kg/ha, between 3 kg/ha and 100 kg/ha, between 4 kg/ha and 100 kg/ha, between 5 kg/ha and 100 kg/ha, between 6 kg/ha and 100 kg/ha, between 7 kg/ha and 100 kg/ha, between 8 kg/ha and 100 kg/ha, between 9 kg/ha and 100 kg/ha, between 10 kg/ha and 100 kg/ha, between 15 kg/ha and 100 kg/ha, between 20 kg/ha and 100 kg/ha, between 30 kg/ha and 100 kg/ha, between 40 kg/ha and 100 kg/ha, between 50 kg/ha and 100 kg/ha, between 75 kg/ha and 100 kg/ha, between 100 kg/ha and 500 kg/ha, between 100 kg/ha and 400 kg/ha, between 100 and 300 kg/ha, or between 100 kg/ha and 200 kg/ha as compared to a population of control tobacco plants when grown under similar growth conditions. As used herein, a “population” of tobacco plants can be of any size for example, 5, 10, 15, 20, 25, 30, 35, 40, 50, 100, 500, 1000, 5000, 10000, 25000, 50000, 100000, 500000, or more. A population can be from a single variety, cultivar, or line. A population can be created using any breeding techniques known in the art.

In an aspect, a modified tobacco plant comprising enhanced NUE provided herein comprises at least 1, at least 2, at least 3, at least 4, at least 5, at least 6, at least 7, at least 8, at least 9, at least 10, at least 11, at least 12, at least 13, at least 14, at least 15, at least 20, or at least 25 more leaves as compared to a control tobacco plant grown under similar growth conditions. In another aspect, a modified tobacco plant comprising enhanced NUE provided herein comprises between 1 and 25, between 2 and 25, between 3 and 25, between 4 and 25, between 5 and 25, between 6 and 25, between 7 and 25, between 8 and 25, between 9 and 25, between 10 and 25, between 11 and 25, between 12 and 25, between 13 and 25, between 14 and 25, between 15 and 25, or between 20 and 25 more leaves as compared to a control tobacco plant grown under similar growth conditions.

As used herein, “comparable conditions” “similar conditions” or “similar growth conditions” refers to similar environmental conditions, agronomic practices, and/or curing process for growing or curing tobacco and making meaningful comparisons between two or more plant genotypes so that neither environmental conditions nor agronomic practices (including curing process) would contribute to, or explain, any differences observed between the two or more plant genotypes. Environmental conditions include, for example, light, temperature, water, humidity, and nutrition (e.g., nitrogen and phosphorus). Agronomic practices include, for example, seeding, clipping, undercutting, transplanting, topping, suckering, and curing. See Chapters 4B and 4C of Tobacco, Production, Chemistry and Technology, Davis & Nielsen, eds., Blackwell Publishing, Oxford (1999), pp. 70-103.

In one aspect, a modified plant, seed, plant part, or plant cell provided herein comprises one or more non-naturally occurring mutations. In one aspect, a mutation provided herein improves nitrogen utilization efficiency in a plant. Types of mutations provided herein include, for example, substitutions (point mutations), deletions, insertions, duplications, and inversions. Such mutations are desirably present in the coding region of a gene; however, mutations in a promoter or other regulatory region, an intron, an intron-exon boundary, or an untranslated region of a gene may also be desirable.

In one aspect, methods provided herein are capable of producing a tobacco plant with enhanced nitrogen utilization efficiency as compared to a control tobacco plant. Mutagenesis methods include, without limitation, chemical mutagenesis, for example, treatment of seeds with ethyl methylsulfate (EMS) (Hildering and Verkerk, In, The use of induced mutations in plant breeding. Pergamon Press, pp. 317-320, 1965); or UV-irradiation, X-rays, electron beams, ion beams (e.g., carbon ion beam, helium ion beam, neon ion beam), and fast neutron irradiation (see, for example, Verkerk, Neth. J. Agric. Sci. 19:197-203, 1971; Poehlman, Breeding Field Crops, Van Nostrand Reinhold, New York (3.sup.rd ed.), 1987; and Tanaka, J. Radiat. Res. 51:223-233, 2010); transposon tagging (Fedoroff et al., 1984; U.S. Pat. Nos. 4,732,856 and 5,013,658); and T-DNA insertion methodologies (Hoekema et al., 1983; U.S. Pat. No. 5,149,645). EMS-induced mutagenesis consists of chemically inducing random point mutations over the length of a genome. Fast neutron mutagenesis consists of exposing seeds to neutron bombardment which causes large deletions through double stranded DNA breakage. Transposon tagging comprises inserting a transposon within an endogenous gene to reduce or eliminate expression of the gene.

In addition, a fast and automatable method for screening for chemically induced mutations, TILLING (Targeting Induced Local Lesions In Genomes), using denaturing high performance liquid chromatography (HPLC) or selective endonuclease digestion of selected PCR products is also applicable to the present disclosure. See, McCallum et al. (2000) Nat. Biotechnol. 18:455-457. Mutations that impact gene expression or that interfere with the function of genes provided herein can be determined using methods that are well known in the art. Insertional mutations in gene exons usually result in null-mutants. Mutations in conserved residues can be particularly effective in inhibiting the function of a protein.

The screening and selection of mutagenized tobacco plants can be through any methodologies known to those having ordinary skill in the art. Examples of screening and selection methodologies include, but are not limited to, Southern blots, PCR amplification for detection of a polynucleotide, Northern blots, RNase protection, primer-extension, RT-PCR amplification for detecting RNA transcripts, Sanger sequencing, Next Generation sequencing technologies (e.g., Illumina, PacBio, Ion Torrent, 454), enzymatic assays for detecting enzyme or ribozyme activity of polypeptides and polynucleotides; and protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides. Methods for performing all of the referenced techniques are known in the art.

In one aspect, a plant genome provided herein is mutated (edited) by a nuclease selected from the group consisting of a meganuclease, a zinc-finger nuclease (ZFN), a transcription activator-like effector nuclease (TALEN), a CRISPR/Cas9 nuclease, a CRISPR/Cpf1, or a CRISPR/Cmx1 nuclease. In another aspect, a plant genome provided herein is mutated by a CRISPR/CasX or a CRISPR/CasY nuclease. As used herein, “editing” or “genome editing” refers to targeted mutagenesis of at least 1, at least 2, at least 3, at least 4, at least 5, at least 6, at least 7, at least 8, at least 9, or at least 10 nucleotides of an endogenous plant genome nucleic acid sequence, or removal or replacement of an endogenous plant genome nucleic acid sequence.

Also provided herein are the transformation of tobacco plants with recombinant constructs or expression cassettes described herein using any suitable transformation methods known in the art. Methods for introducing polynucleotide sequences into tobacco plants are known in the art and include, but are not limited to, stable transformation methods, transient transformation methods, and virus-mediated methods. “Stable transformation” refers to transformation where the nucleotide construct of interest introduced into a plant integrates into a genome of a plant cell and is capable of being inherited by the progeny thereof. “Transient transformation” is intended to mean that a sequence is introduced into a plant or plant cell and is only temporally expressed or is only transiently present in the plant or plant cell.

In one aspect, methods and compositions provided herein comprise the introduction of one or more polynucleotides into one or more plant cells. In one aspect, a plant genome provided herein is modified to include an introduced polynucleotide or recombinant DNA construct. As used herein, “plant genome” refers to a nuclear genome, a mitochondrial genome, or a plastid (e.g., chloroplast) genome of a plant cell. In another aspect, a polynucleotide provided herein is integrated into an artificial chromosome. In one aspect, an artificial chromosome comprising a polynucleotide provided herein is integrated into a plant cell.

In one aspect, a modified plant, seed, plant component, plant cell, or plant genome provided herein comprises one or more transgenes. In one aspect, a transgene provided herein improves nitrogen utilization efficiency in a tobacco plant. As used herein, a “transgene” refers to a polynucleotide that has been transferred into a genome by any method known in the art. In one aspect, a transgene is an exogenous polynucleotide. In one aspect, a transgene is an endogenous polynucleotide that is integrated into a new genomic locus where it is not normally found. Therefore, a transgene can also be a cisgene under appropriate circumstances.

In one aspect, transgenes provided herein comprise a recombinant DNA construct. In one aspect, recombinant DNA constructs or expression cassettes provided herein can comprise a selectable marker gene for the selection of transgenic cells. Selectable marker genes include, but are not limited to, genes encoding antibiotic resistance, such as those encoding neomycin phosphotransferase II (NPTII) and hygromycin phosphotransferase (HPT), as well as genes conferring resistance to herbicidal compounds, such as glufosinate ammonium, bromoxynil, imidazolinones, triazolopyrimidines, sulfonylurea (e.g., chlorsulfuron and sulfometuron methyl), and 2,4-dichlorophenoxyacetate (2,4-D). Additional selectable markers include phenotypic markers such as β-galactosidase and fluorescent proteins such as green fluorescent protein (GFP).

In one aspect, methods and compositions provided herein comprise a vector. As used herein, the terms “vector” or “plasmid” are used interchangeably and refer to a circular, double-stranded DNA molecule that is physically separate from chromosomal DNA. In one aspect, a plasmid or vector used herein is capable of replication in vivo. A “transformation vector,” as used herein, is a plasmid that is capable of transforming a plant cell. In an aspect, a plasmid provided herein is a bacterial plasmid. In another aspect, a plasmid provided herein is an Agrobacterium Ti plasmid or derived from an Agrobacterium Ti plasmid. In still another aspect, a vector provided herein is a viral vector.

In one aspect, a plasmid or vector provided herein is a recombinant vector. As used herein, the term “recombinant vector” refers to a vector formed by laboratory methods of genetic recombination, such as molecular cloning. In another aspect, a plasmid provided herein is a synthetic plasmid. As used herein, a “synthetic plasmid” is an artificially created plasmid that is capable of the same functions (e.g., replication) as a natural plasmid (e.g., Ti plasmid). Without being limited, one skilled in the art can create a synthetic plasmid de novo via synthesizing a plasmid by individual nucleotides, or by splicing together nucleic acid molecules from different pre-existing plasmids.

Vectors are commercially available or can be produced by recombinant DNA techniques routine in the art. In one aspect, a vector provided herein comprises all or part of SEQ ID NO: 65. A vector containing a nucleic acid can have expression elements operably linked to such a nucleic acid, and further can include sequences such as those encoding a selectable marker (e.g., an antibiotic resistance gene). A vector containing a nucleic acid can encode a chimeric or fusion polypeptide (i.e., a polypeptide operatively linked to a heterologous polypeptide, which can be at either the N-terminus or C-terminus of the polypeptide). Representative heterologous polypeptides are those that can be used in purification of the encoded polypeptide (e.g., 6×His tag, glutathione S-transferase (GST)).

Suitable methods of introducing polynucleotides (e.g., transgenes, recombinant vectors, recombinant DNA constructs, expression constructs) into plant cells of the present disclosure include microinjection (Crossway et al. (1986) Biotechniques 4:320-334), electroporation (Shillito et al. (1987) Meth. Enzymol. 153:313-336; Riggs et al. (1986) Proc. Natl. Acad. Sci. USA 83:5602-5606), Agrobacterium-mediated transformation (U.S. Pat. Nos. 5,104,310, 5,149,645, 5,177,010, 5,231,019, 5,463,174, 5,464,763, 5,469,976, 4,762,785, 5,004,863, 5,159,135, 5,563,055, and 5,981,840), direct gene transfer (Paszkowski et al. (1984) EMBO J. 3:2717-2722), and ballistic particle acceleration (see, for example, U.S. Pat. Nos. 4,945,050, 5,141,131, 5,886,244, 5,879,918, and 5,932,782; Tomes et al. (1995) in Plant Cell, Tissue, and Organ Culture Fundamental Methods, ed. Gamborg and Phillips (Springer-Verlag, Berlin); McCabe et al. (1988) Biotechnology 6:923-926). Also see Weissinger et al. (1988) Ann. Rev. Genet. 22:421-477; Christou et al. (1988) Plant Physiol. 87:671-674 (soybean); McCabe et al. (1988) Bio Technology 6:923-926 (soybean); Finer and McMullen (1991) In Vitro Cell Dev. Biol. 27P: 175-182 (soybean); Singh et al. (1998) Theor. Appl. Genet. 96:319-324 (soybean); De Wet et al. (1985) in The Experimental Manipulation of Ovule Tissues, ed. Chapman et al. (Longman, N.Y.), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation). In one aspect, a bacterial cell provided herein comprises a recombinant DNA construct or recombinant vector provided herein. It is appreciated that many different species of bacterial cells can comprise a recombinant DNA construct or recombinant vector, I including, but not limited to, Agrobacterium tumefaciens, Escherichia coli. Yeast cells (e.g., Saccharomyces cerevisiae) comprising a recombinant DNA construct or recombinant vector provided herein are also provided.

In another aspect, recombinant constructs or expression cassettes provided herein may be introduced into plants by contacting plants with a virus or viral nucleic acids. Generally, such methods involve incorporating an expression cassette of the present disclosure within a viral DNA or RNA molecule. It is recognized that promoters for use in the expression cassettes provided herein also encompass promoters utilized for transcription by viral RNA polymerases. Methods for introducing polynucleotides into plants and expressing a protein encoded therein, involving viral DNA or RNA molecules, are known in the art. See, for example, U.S. Pat. Nos. 5,889,191, 5,889,190, 5,866,785, 5,589,367, 5,316,931, and Porta et al. (1996) Molecular Biotechnology 5:209-221.

Any plant tissue that can be subsequently propagated using clonal methods, whether by organogenesis or embryogenesis, may be transformed with a recombinant construct or an expression cassette provided herein. By “organogenesis” in intended the process by which shoots and roots are developed sequentially from meristematic centers. By “embryogenesis” is intended the process by which shoots and roots develop together in a concerted fashion (not sequentially), whether from somatic cells or gametes. Exemplary tissues that are suitable for various transformation protocols described herein include, but are not limited to, callus tissue, existing meristematic tissue (e.g., apical meristems, axillary buds, and root meristems) and induced meristem tissue (e.g., cotyledon meristem and hypocotyl meristem), hypocotyls, cotyledons, leaf disks, pollen, embryos, and the like.

As commonly understood in the art, the term “promoter” may generally refer to a DNA sequence that contains an RNA polymerase binding site, transcription start site, and/or TATA box and assists or promotes the transcription and expression of an associated transcribable polynucleotide sequence and/or gene (or transgene). A promoter may be synthetically produced, varied or derived from a known or naturally occurring promoter sequence or other promoter sequence (e.g., as provided herein). A promoter may also include a chimeric promoter comprising a combination of two or more heterologous sequences. A promoter of the present invention may thus include variants of promoter sequences that are similar in composition, but not identical to or complimentary to, other promoter sequence(s) known or provided herein. As used herein, a “heterologous promoter” in the context of a DNA construct refers to either: (i) a promoter that is derived from a source distinct from the operably linked structural gene or coding region or (ii) a promoter derived from the same source as the operably linked structural gene or coding region, where the promoter's sequence is modified from its original form. As used herein, the term “operably linked” refers to a functional linkage between a promoter or other regulatory element and an associated transcribable polynucleotide sequence or coding sequence of a gene (or transgene), such that the promoter, etc., operates to initiate, assist, affect, cause, and/or promote the transcription and expression of the associated coding or transcribable polynucleotide sequence, at least in particular tissue(s), developmental stage(s), and/or under certain condition(s). A “plant expressible promoter” refers to a promoter that may be used to express in a plant, plant cell and/or plant tissue an associated coding sequence, transgene or transcribable polynucleotide sequence that is operably linked to the promoter.

A promoter may be classified according to a variety of criteria relating to the pattern of expression of a coding sequence or gene (including a transgene) operably linked to the promoter, such as constitutive, developmental, tissue-specific, inducible, etc. Promoters that initiate transcription in all or most tissues of the plant are referred to as “constitutive” promoters. Promoters that initiate transcription during certain periods or stages of development are referred to as “developmental” promoters. Promoters whose expression is enhanced in certain tissues of the plant relative to other plant tissues are referred to as “tissue-enhanced” or “tissue-preferred” promoters. Thus, a “tissue-preferred” promoter causes relatively higher or preferential expression in a specific tissue(s) of the plant, but with lower levels of expression in other tissue(s) of the plant. Promoters that express within a specific tissue(s) of the plant, with little or no expression in other plant tissues, are referred to as “tissue-specific” promoters. A promoter that expresses in a certain cell type of the plant is referred to as a “cell type specific” promoter. An “inducible” promoter is a promoter that initiates transcription in response to an environmental stimulus such as cold, drought or light, or other stimuli, such as wounding or chemical application. A promoter may also be classified in terms of its origin, such as being heterologous, homologous, chimeric, synthetic, etc. A “heterologous” promoter is a promoter sequence having a different origin relative to its associated transcribable sequence, coding sequence, or gene (or transgene), and/or not naturally occurring in the plant species to be transformed. The term “heterologous” may refer more broadly to a combination of two or more DNA molecules or sequences when such a combination is not normally found in nature. For example, two or more DNA molecules or sequences would be heterologous with respect to each other if they are normally found in different genomes or at different loci in the same genome, or if they are not identically combined in nature.

Exemplary constitutive promoters include the core promoter of the Rsyn7 promoter and other constitutive promoters disclosed in U.S. Pat. No. 6,072,050; the core CaMV 35S promoter (Odell et al. (1985) Nature 313:810-812); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J 3:2723-2730); ALS promoter (U.S. Pat. No. 5,659,026), and the like.

Exemplary chemical-inducible promoters include the tobacco PR-la promoter, which is activated by salicylic acid. Other chemical-inducible promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible promoters (see, for example, Gatz et al. (1991) Mol. Gen. Genet. 227:229-237, and U.S. Pat. Nos. 5,814,618 and 5,789,156). Additional exemplary promoters that can be used herein are those responsible for heat-regulated gene expression, light-regulated gene expression (for example, the pea rbcS-3A; the maize rbcS promoter; the chlorophyll alb-binding protein gene found in pea; or the Arabssu promoter), hormone-regulated gene expression (for example, the abscisic acid (ABA) responsive sequences from the Em gene of wheat; the ABA-inducible HVA1 and HVA22, and rd29A promoters of barley and Arabidopsis; and wound-induced gene expression (for example, of wunl), organ specific gene expression (for example, of the tuber-specific storage protein gene; the 23-kDa zein gene from maize described by; or the French bean (8-phaseolin gene), or pathogen-inducible promoters (for example, the PR-1, prp-1, or (8-1,3 glucanase promoters, the fungal-inducible wirla promoter of wheat, and the nematode-inducible promoters, TobRB7-5A and Hmg-1, of tobacco arid parsley, respectively).

As used herein, a “leaf” promoter includes any promoter that initiates, causes, drives, etc., transcription or expression of its associated gene, transgene or transcribable DNA sequence in leaf tissue derived from any part of a plant. Such a “leaf” promoter may be further defined as initiating, causing, driving, etc., transcription or expression of its associated gene/transgene or transcribable DNA sequence in one or more tissue(s) of a plant, such as one or more floral tissue(s). Such a “leaf” promoter may be further defined as a “leaf preferred” promoter that initiates, causes, drives, etc., transcription or expression of its associated gene, transgene or transcribable DNA sequence at least preferentially or mostly, if not exclusively, in leaf tissue derived from any part of a plant (as opposed to floral tissue). However, a “leaf” and a “leaf preferred” promoter may each also permit, allow, cause, drive, etc., transcription or expression of its associated gene, transgene or transcribable DNA sequence during reproductive phase(s) or stage(s) of development in one or more cells or tissues of the plant, such as in one or more vegetative or reproductive tissue(s). In fact, a “leaf” promoter may even initiate, cause, drive, etc., transcription or expression of its associated gene, transgene or transcribable DNA sequence in one or more reproductive or vegetative tissues at a greater level or extent than in leaf tissue(s).

As used herein, a “root” promoter includes any promoter that initiates, causes, drives, etc., transcription or expression of its associated gene, transgene or transcribable DNA sequence in root tissue derived from any part of a plant. Such a “root” promoter may be further defined as initiating, causing, driving, etc., transcription or expression of its associated gene/transgene or transcribable DNA sequence in one or more tissue(s) of a plant, such as one or more floral tissue(s). Such a “root” promoter may be further defined as a “root preferred” promoter that initiates, causes, drives, etc., transcription or expression of its associated gene, transgene or transcribable DNA sequence at least preferentially or mostly, if not exclusively, in root tissue derived from any part of a plant (as opposed to floral tissue). However, a “root” and a “root preferred” promoter may each also permit, allow, cause, drive, etc., transcription or expression of its associated gene, transgene or transcribable DNA sequence during reproductive phase(s) or stage(s) of development in one or more cells or tissues of the plant, such as in one or more vegetative or reproductive tissue(s). In fact, a “root” promoter may even initiate, cause, drive, etc., transcription or expression of its associated gene, transgene or transcribable DNA sequence in one or more reproductive or vegetative tissues at a greater level or extent than in root tissue(s).

Additional exemplary tissue-preferred promoters include those disclosed in Yamamoto et al. (1997) Plant J. 12(2):255-265; Kawamata et al. (1997) Plant Cell Physiol. 38(7):792-803; Hansen et al. (1997) Mol. Gen. Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol. Biol. 23(6):1129-1138; Matsuoka et al. (1993) Proc Natl. Acad. Sci. USA 90(20):9586-9590; and Guevara-Garcia et al. (1993) Plant J. 4(3):495-505.

As used herein, “operably linked” refers to a functional linkage between two or more elements. For example, an operable linkage between a polynucleotide of interest and a regulatory sequence (e.g., a promoter) is a functional link that allows for expression of the polynucleotide of interest. Operably linked elements may be contiguous or non-contiguous.

As used herein, “heterologous” refers to a sequence that originates from a foreign species, or, if from the same species, is substantially modified from its native form in composition and/or genomic locus by deliberate human intervention. The term also is applicable to nucleic acid constructs, also referred to herein as “polynucleotide constructs” or “nucleotide constructs.” In this manner, a “heterologous” nucleic acid construct is intended to mean a construct that originates from a foreign species, or, if from the same species, is substantially modified from its native form in composition and/or genomic locus by deliberate human intervention. Heterologous nucleic acid constructs include, but are not limited to, recombinant nucleotide constructs that have been introduced into a plant or plant part thereof, for example, via transformation methods or subsequent breeding of a transgenic plant with another plant of interest.

In one aspect, inhibition of the expression of one or more polypeptides provided herein may be obtained by RNA interference (RNAi) by expression of a polynucleotide provided herein. In one aspect, RNAi comprises expressing a non-coding RNA. As used herein, a “non-coding RNA” is selected from the group consisting of a microRNA (miRNA), a small interfering RNA (siRNA), a trans-acting siRNA (ta-siRNA), a transfer RNA (tRNA), a ribosomal RNA (rRNA), an intron, a hairpin RNA (hpRNA), an intron-containing hairpin RNA (ihpRNA), and guide RNA. In one aspect, a single non-coding RNA provided herein inhibits the expression of at least 1, at least 2, at least 3, at least 4, at least 5, at least 6, at least 7, at least 8, at least 9, at least 10, or more than 10 polypeptides. In one aspect, a non-coding RNA provided herein is stably transformed into a plant genome. In another aspect, a non-coding RNA provided herein is transiently transformed into a plant genome.

As used herein, the terms “down-regulate,” “suppress,” “inhibit,” “inhibition,” and “inhibiting” are defined as any method known in the art or described herein that decreases the expression or function of a gene product of interest (e.g., an mRNA, a protein, a non-coding RNA). “Inhibition” can be in the context of a comparison between two plants, for example, a modified plant versus a control plant. Alternatively, inhibition of expression or function of a target gene product can be in the context of a comparison between plant cells, organelles, organs, tissues, or plant components within the same plant or between different plants, and includes comparisons between developmental or temporal stages within the same plant or plant component or between plants or plant components. “Inhibition” includes any relative decrement of function or production of a gene product of interest, up to and including complete elimination of function or production of that gene product. The term “inhibition” encompasses any method or composition that down-regulates translation and/or transcription of the target gene product or functional activity of the target gene product.

The term “inhibitory sequence” encompasses any polynucleotide or polypeptide sequence capable of inhibiting the expression or function of a gene in a plant, such as full-length polynucleotide or polypeptide sequences, truncated polynucleotide or polypeptide sequences, fragments of polynucleotide or polypeptide sequences, variants of polynucleotide or polypeptide sequences, sense-oriented nucleotide sequences, antisense-oriented nucleotide sequences, the complement of a sense- or antisense-oriented nucleotide sequence, inverted regions of nucleotide sequences, hairpins of nucleotide sequences, double-stranded nucleotide sequences, single-stranded nucleotide sequences, combinations thereof, and the like. The term “polynucleotide sequence” includes sequences of RNA, DNA, chemically modified nucleic acids, nucleic acid analogs, combinations thereof, and the like.

When the phrase “capable of inhibiting” is used in the context of a polynucleotide inhibitory sequence, it is intended to mean that the inhibitory sequence itself exerts the inhibitory effect; or, where the inhibitory sequence encodes an inhibitory nucleotide molecule (for example, hairpin RNA, miRNA, or double-stranded RNA polynucleotides), or encodes an inhibitory polypeptide (e.g., a polypeptide that inhibits expression or function of the target gene product), following its transcription (for example, in the case of an inhibitory sequence encoding a hairpin RNA, miRNA, or double-stranded RNA polynucleotide) or its transcription and translation (in the case of an inhibitory sequence encoding an inhibitory polypeptide), the transcribed or translated product, respectively, exerts the inhibitory effect on the target gene product (e.g., inhibits expression or function of the target gene product).

An inhibitory sequence provided herein can be a sequence triggering gene silencing via any silencing pathway or mechanism known in the art, including, but not limited to, sense suppression/co-suppression, antisense suppression, double-stranded RNA (dsRNA) interference, hairpin RNA interference and intron-containing hairpin RNA interference, amplicon-mediated interference, ribozymes, small interfering RNA, artificial or synthetic microRNA, and artificial trans-acting siRNA. An inhibitory sequence may range from at least 20 nucleotides, at least 50 nucleotides, at least 70 nucleotides, at least 100 nucleotides, at least 150 nucleotides, at least 200 nucleotides, at least 250 nucleotides, at least 300 nucleotides, at least 350 nucleotides, at least 400 nucleotides, and up to the full-length polynucleotide encoding the proteins of the present disclosure, depending upon the desired outcome. In one aspect, an inhibitory sequence can be a fragment of between 50 and 400 nucleotides, between 70 and 350 nucleotides, between 90 and 325 nucleotides, between 90 and 300 nucleotides, between 90 and 275 nucleotides, between 100 and 400 nucleotides, between 100 and 350 nucleotides, between 100 and 325 nucleotides, between 100 and 300 nucleotides, between 125 and 300 nucleotides, or between 125 and 275 nucleotides in length.

MicroRNAs (miRNAs) are non-protein coding RNAs, generally of between 19 to 25 nucleotides (commonly 20 to 24 nucleotides in plants), that guide cleavage in trans of target transcripts, negatively regulating the expression of genes involved in various regulation and development pathways (Bartel (2004) Cell, 116:281-297). In some cases, miRNAs serve to guide in-phase processing of siRNA primary transcripts (see Allen et al. (2005) Cell, 121:207-221).

Many microRNA genes (MIR genes) have been identified and made publicly available in a database (“miRBase”, available on line at microrna.sanger.ac.uk/sequences; also see Griffiths-Jones et al. (2003) Nucleic Acids Res., 31:439-441). MIR genes have been reported to occur in intergenic regions, both isolated and in clusters in the genome, but can also be located entirely or partially within introns of other genes (both protein-coding and non-protein-coding). Transcription of MIR genes can be, at least in some cases, under promotional control of a MIR gene's own promoter. The primary transcript, termed a “pri-miRNA”, can be quite large (several kilobases) and can be polycistronic, containing one or more pre-miRNAs (fold-back structures containing a stem-loop arrangement that is processed to the mature miRNA) as well as the usual 5′ “cap” and polyadenylated tail of an mRNA.

Maturation of a mature miRNA from its corresponding precursors (pri-miRNAs and pre-miRNAs) differs significantly between animals and plants. For example, in plant cells, microRNA precursor molecules are believed to be largely processed to the mature miRNA entirely in the nucleus, whereas in animal cells, the pri-miRNA transcript is processed in the nucleus by the animal-specific enzyme Drosha, followed by export of the pre-miRNA to the cytoplasm where it is further processed to the mature miRNA. Mature miRNAs in plants are typically 21 nucleotides in length.

Transgenic expression of miRNAs (whether a naturally occurring sequence or an artificial sequence) can be employed to regulate expression of the miRNA's target gene or genes. Inclusion of a miRNA recognition site in a transgenically expressed transcript is also useful in regulating expression of the transcript; see, for example, Parizotto et al. (2004) Genes Dev., 18:2237-2242. Recognition sites of miRNAs have been validated in all regions of an mRNA, including the 5′ untranslated region, coding region, and 3′ untranslated region, indicating that the position of the miRNA target site relative to the coding sequence may not necessarily affect suppression. Because miRNAs are important regulatory elements in eukaryotes, transgenic suppression of miRNAs is useful for manipulating biological pathways and responses. Finally, promoters of MIR genes can have very specific expression patterns (e.g., cell-specific, tissue-specific, temporally specific, or inducible), and thus are useful in recombinant constructs to induce such specific transcription of a DNA sequence to which they are operably linked. Various utilities of miRNAs, their precursors, their recognition sites, and their promoters are known. Non-limiting examples of these utilities include: (1) the expression of a native miRNA or miRNA precursor sequence to suppress a target gene; (2) the expression of an artificial miRNA or miRNA precursor sequence to suppress a target gene; (3) expression of a transgene with a miRNA recognition site, where the transgene is suppressed when the mature miRNA is expressed; (4) expression of a transgene driven by a miRNA promoter.

Designing an artificial miRNA sequence can be as simple as substituting sequence that is complementary to the intended target for nucleotides in the miRNA stem region of the miRNA precursor. One non-limiting example of a general method for determining nucleotide changes in the native miRNA sequence to produce the engineered miRNA precursor includes the following steps: (a) Selecting a unique target sequence of at least 18 nucleotides specific to the target gene, e.g., by using sequence alignment tools such as BLAST© (see, for example, Altschul et al. (1990) J. Mol. Biol., 215:403-410; Altschul et al. (1997) Nucleic Acids Res., 25:3389-3402), for example, of both tobacco cDNA and genomic DNA databases, to identify target transcript orthologues and any potential matches to unrelated genes, thereby avoiding unintentional silencing of non-target sequences; (b) Analyzing the target gene for undesirable sequences (e.g., matches to sequences from non-target species), and score each potential 19-mer segment for GC content, Reynolds score (see Reynolds et al. (2004) Nature Biotechnol., 22:326-330), and functional asymmetry characterized by a negative difference in free energy (“.DELTA..DELTA.G” or “ΔΔG”). Preferably 19-mers are selected that have all or most of the following characteristics: (1) a Reynolds score>4, (2) a GC content between 40% to 60%, (3) a negative ΔΔG, (4) a terminal adenosine, (5) lack of a consecutive run of 4 or more of the same nucleotide; (6) a location near the 3′ terminus of the target gene; (7) minimal differences from the miRNA precursor transcript. Positions at every third nucleotide in an siRNA have been reported to be especially important in influencing RNAi efficacy and an algorithm, “siExplorer” is publicly available at rna.chem.t.u-tokyo.ac.jp/siexplorer.htm; (c) Determining the reverse complement of the selected 19-mers to use in making a modified mature miRNA. The additional nucleotide at position 20 is preferably matched to the selected target sequence, and the nucleotide at position 21 is preferably chosen to either be unpaired to prevent spreading of silencing on the target transcript or paired to the target sequence to promote spreading of silencing on the target transcript; and (d) transforming the artificial miRNA into a plant.

In one aspect, an artificial miRNA provided herein reduces or eliminates RNA transcription or protein translation of a target gene.

In one aspect, a miRNA or an artificial miRNA provided herein is under the control of a tissue specific promoter. In a further aspect, a miRNA or an artificial miRNA provided herein is under the control of a tissue-preferred promoter. In a further aspect, a miRNA or an artificial miRNA provided herein is under the control of a constitutive promoter.

Tobacco material obtained from modified tobacco lines, varieties or hybrids of the present disclosure can be used to make tobacco products. As used herein, “tobacco product” is defined as any product made or derived from tobacco that is intended for human use or consumption. In an aspect, a tobacco product provided herein comprises cured components from a modified tobacco plant provided herein. In another aspect, a tobacco product provided herein comprises cured tobacco leaves from a modified tobacco plant provided herein.

Tobacco products provided herein include, without limitation, cigarette products (e.g., cigarettes, bidi cigarettes, kreteks), cigar products (e.g., cigars, cigar wrapping tobacco, cigarillos), pipe tobacco products, products derived from tobacco, tobacco-derived nicotine products, smokeless tobacco products (e.g., moist snuff, dry snuff, chewing tobacco, moist smokeless tobacco, fine cut chewing tobacco, long cut chewing tobacco, pouched chewing tobacco), films, chewables (e.g., gum), lozenges, dissolving strips, tabs, tablets, shaped parts, gels, consumable units, insoluble matrices, hollow shapes, reconstituted tobacco, expanded tobacco, and the like. See, for example, U.S. Patent Publication No. US 2006/0191548.

As used herein, “cigarette” refers a tobacco product having a “rod” and “filler”. The cigarette “rod” includes the cigarette paper, filter, plug wrap (used to contain filtration materials), tipping paper that holds the cigarette paper (including the filler) to the filter, and all glues that hold these components together. The “filler” includes (1) all tobaccos, including but not limited to reconstituted and expanded tobacco, (2) non-tobacco substitutes (including but not limited to herbs, non-tobacco plant materials and other spices that may accompany tobaccos rolled within the cigarette paper), (3) casings, (4) flavorings, and (5) all other additives (that are mixed into tobaccos and substitutes and rolled into the cigarette).

In one aspect, this disclosure provides nicotine derived from and a method of producing nicotine from a modified tobacco plant provided herein for use in a product.

Tobacco products derived from plants of the present disclosure also include cigarettes and other smoking articles, particularly those smoking articles including filter elements, where the rod of smokeable material includes cured tobacco within a tobacco blend. In an aspect, a tobacco product of the present disclosure is selected from the group consisting of a cigarillo, a non-ventilated recess filter cigarette, a vented recess filter cigarette, a bidi cigarette, a cigar, snuff, pipe tobacco, cigar tobacco, cigarette tobacco, chewing tobacco, leaf tobacco, hookah tobacco, shredded tobacco, and cut tobacco. In another aspect, a tobacco product of the present disclosure is a smokeless tobacco product. Smokeless tobacco products are not combusted and include, but not limited to, chewing tobacco, moist smokeless tobacco, snus, and dry snuff. Chewing tobacco is coarsely divided tobacco leaf that is typically packaged in a large pouch-like package and used in a plug or twist. Moist smokeless tobacco is a moist, more finely divided tobacco that is provided in loose form or in pouch form and is typically packaged in round cans and used as a pinch or in a pouch placed between an adult tobacco consumer's cheek and gum. Snus is a heat treated smokeless tobacco. Dry snuff is finely ground tobacco that is placed in the mouth or used nasally. In a further aspect, a tobacco product of the present disclosure is selected from the group consisting of loose leaf chewing tobacco, plug chewing tobacco, moist snuff, and nasal snuff. In yet another aspect, a tobacco product of the present disclosure is selected from the group consisting of an electronically heated cigarette, an e-cigarette, an electronic vaporing device. In one aspect, a method provided herein comprises preparing a tobacco product using a cured tobacco leaf from a modified tobacco plant provided herein.

As used herein, “reconstituted tobacco” refers to a part of tobacco filler made from tobacco dust and other tobacco scrap material, processed into sheet form and cut into strips to resemble tobacco. In addition to the cost savings, reconstituted tobacco is very important for its contribution to cigarette taste from processing flavor development using reactions between ammonia and sugars.

As used herein, “expanded tobacco” refers to a part of tobacco filler which is processed through expansion of suitable gases so that the tobacco is “puffed” resulting in reduced density and greater filling capacity. It reduces the weight of tobacco used in cigarettes.

Also provided herein is cured tobacco material made from tobacco plants or plant components provided herein. “Curing” is the aging process that reduces moisture and brings about the destruction of chlorophyll giving tobacco leaves a golden color and by which starch is converted to sugar. Cured tobacco therefore has a higher reducing sugar content and a lower starch content compared to harvested green leaf. In one aspect, tobacco plants or plant components provided herein can be cured using conventional means, e.g., flue-cured, barn-cured, fire-cured, air-cured or sun-cured. See, for example, Tso (1999, Chapter 1 in Tobacco, Production, Chemistry and Technology, Davis & Nielsen, eds., Blackwell Publishing, Oxford) for a description of different types of curing methods. Cured tobacco is usually aged in a wooden drum (e.g., a hogshead) or cardboard cartons in compressed conditions for several years (e.g., two to five years), at a moisture content ranging from 10% to 25%. See, U.S. Pat. Nos. 4,516,590 and 5,372,149. Cured and aged tobacco then can be further processed. Further processing includes conditioning the tobacco under vacuum with or without the introduction of steam at various temperatures, pasteurization, and fermentation. Fermentation typically is characterized by high initial moisture content, heat generation, and a 10 to 20% loss of dry weight. See, for example, U.S. Pat. Nos. 4,528,993, 4,660,577, 4,848,373, 5,372,149; U.S. Publication No. 2005/0178398; and Tso (1999, Chapter 1 in Tobacco, Production, Chemistry and Technology, Davis & Nielsen, eds., Blackwell Publishing, Oxford). Cured, aged, and fermented tobacco can be further processed (e.g., cut, shredded, expanded, or blended). See, for example, U.S. Pat. Nos. 4,528,993; 4,660,577; and 4,987,907. In one aspect, the cured tobacco material of the present disclosure is flue-cured, sun-cured, air-cured, or fire-cured.

The present disclosure further provides a method manufacturing a tobacco product comprising tobacco material from tobacco plants provided herein. In one aspect, methods provided herein comprise conditioning aged tobacco material made from tobacco plants provided herein to increase its moisture content from between 12.5% and 13.5% to 21%, blending the conditioned tobacco material to produce a desirable blend. In one aspect, the method of manufacturing a tobacco product provided herein further comprises casing or flavoring the blend. Generally, during the casing process, casing or sauce materials are added to blends to enhance their quality by balancing the chemical composition and to develop certain desired flavor characteristics. Further details for the casing process can be found in Tobacco Production, Chemistry and Technology, Edited by L. Davis and M. Nielsen, Blackwell Science, 1999.

Tobacco material provided herein can be also processed using methods including, but not limited to, heat treatment (e.g., cooking, toasting), flavoring, enzyme treatment, expansion and/or curing. Both fermented and non-fermented tobaccos can be processed using these techniques. Examples of suitable processed tobaccos include dark air-cured, dark fire cured, Burley, flue cured, and cigar filler or wrapper, as well as the products from the whole leaf stemming operation. In one aspect, tobacco fibers include up to 70% dark tobacco on a fresh weight basis. For example, tobacco can be conditioned by heating, sweating and/or pasteurizing steps as described in U.S. Publication Nos. 2004/0118422 or 2005/0178398.

Tobacco material provided herein can be subject to fermentation. Fermenting typically is characterized by high initial moisture content, heat generation, and a 10 to 20% loss of dry weight. See, e.g., U.S. Pat. Nos. 4,528,993; 4,660,577; 4,848,373; and 5,372,149. In addition to modifying the aroma of the leaf, fermentation can change either or both the color and texture of a leaf. Also during the fermentation process, evolution gases can be produced, oxygen can be taken up, the pH can change, and the amount of water retained can change. See, for example, U.S. Publication No. 2005/0178398 and Tso (1999, Chapter 1 in Tobacco, Production, Chemistry and Technology, Davis & Nielsen, eds., Blackwell Publishing, Oxford). Cured, or cured and fermented tobacco can be further processed (e.g., cut, expanded, blended, milled or comminuted) prior to incorporation into the oral product. The tobacco, in some cases, is long cut fermented cured moist tobacco having an oven volatiles content of between 48 and 50 weight percent prior to mixing with a copolymer and, optionally, flavorants and other additives.

In one aspect, tobacco material provided herein can be processed to a desired size. In certain aspects, tobacco fibers can be processed to have an average fiber size of less than 200 micrometers. In one aspect, tobacco fibers are between 75 and 125 micrometers. In another aspect, tobacco fibers are processed to have a size of 75 micrometers or less. In one aspect, tobacco fibers include long cut tobacco, which can be cut or shredded into widths of 10 cuts/inch up to 110 cuts/inch and lengths of 0.1 inches up to 1 inch. Double cut tobacco fibers can have a range of particle sizes such that 70% of the double cut tobacco fibers falls between the mesh sizes of −20 mesh and 80 mesh.

Tobacco material provided herein can be processed to have a total oven volatiles content of 10% by weight or greater; 20% by weight or greater; 40% by weight or greater; 15% by weight to 25% by weight; 20% by weight to 30% by weight; 30% by weight to 50% by weight; 45% by weight to 65% by weight; or 50% by weight to 60% by weight. Those of skill in the art will appreciate that “moist” tobacco typically refers to tobacco that has an oven volatiles content of between 40% by weight and 60% by weight (e.g., 45% by weight to 55% by weight, or 50% by weight). As used herein, “oven volatiles” are determined by calculating the percentage of weight loss for a sample after drying the sample in a pre-warmed forced draft oven at 110° C. for 3.25 hours. An oral product can have a different overall oven volatiles content than the oven volatiles content of the tobacco fibers used to make the oral product. The processing steps described herein can reduce or increase the oven volatiles content.

In one aspect, tobacco plants, seeds, plant components, plant cells, and plant genomes provided herein are from a tobacco type selected from the group consisting of flue-cured tobacco, sun-cured tobacco, air-cured tobacco, dark air-cured tobacco, and dark fire-cured tobacco. In another aspect, tobacco plants, seeds, plant components, plant cells, and plant genomes provided herein are from a tobacco type selected from the group consisting of Burley tobacco, Maryland tobacco, bright tobacco, Virginia tobacco, Oriental tobacco, Turkish tobacco, and Galpão tobacco. In one aspect, a tobacco plants or seed provided herein is a hybrid plants or seed. As used herein, a “hybrid” is created by crossing two plants from different varieties or species, such that the progeny comprises genetic material from each parent. Skilled artisans recognize that higher order hybrids can be generated as well. For example, a first hybrid can be made by crossing Variety C with Variety D to create a C×D hybrid, and a second hybrid can be made by crossing Variety E with Variety F to create an E×F hybrid. The first and second hybrids can be further crossed to create the higher order hybrid (C×D)×(E×F) comprising genetic information from all four parent varieties.

Flue-cured tobaccos (also called Virginia of bright tobaccos) amount to approximately 40% of world tobacco production. Flue-cured tobaccos are often also referred to as “bright tobacco” because of the golden-yellow to deep-orange color it reaches during curing. Flue-cured tobaccos have a light, bright aroma and taste. Flue-cured tobaccos are generally high in sugar and low in oils. Major flue-cured tobacco growing countries are Argentina, Brazil, China, India, Tanzania and the U.S. In one aspect, modified tobacco plants or seeds provided herein are in a flue-cured tobacco background selected from the group consisting of CC 13, CC 27, CC 33, CC35, CC 37, CC 65, CC 67, CC 700, GF 318, GL 338, GL 368, GL 939, K 346, K 399, K326, NC 102, NC 196, NC 291, NC 297, NC 299, NC 471, NC 55, NC 606, NC 71, NC 72, NC 92, PVH 1118, PVH 1452, PVH 2110, SPEIGHT 168, SPEIGHT 220, SPEIGHT 225, SPEIGHT 227, SPEIGHT 236, and any variety essentially derived from any one of the foregoing varieties. In another aspect, modified tobacco plants or seeds provided herein are in a flue-cured tobacco background selected from the group consisting of Coker 48, Coker 176, Coker 371-Gold, Coker 319, Coker 347, GL 939, K 149, K326, K 340, K 346, K 358, K 394, K 399, K 730, NC 27NF, NC 37NF, NC 55, NC 60, NC 71, NC 72, NC 82, NC 95, NC 297, NC 606, NC 729, NC 2326, McNair 373, McNair 944, Ox 207, Ox 414 NF, Reams 126, Reams 713, Reams 744, RG 8, RG 11, RG 13, RG 17, RG 22, RG 81, RG H4, RG H51, Speight H-20, Speight G-28, Speight G-58, Speight G-70, Speight G-108, Speight G-111, Speight G-117, Speight 168, Speight 179, Speight NF-3, Va 116, Va 182, and any variety essentially derived from any one of the foregoing varieties. See WO 2004/041006 A1. In further aspects, modified tobacco plants, seeds, hybrids, varieties, or lines provided herein are in any flue cured background selected from the group consisting of K326, K346, and NC196.

Air-cured tobaccos include Burley, Maryland, and dark tobaccos. The common factor is that curing is primarily without artificial sources of heat and humidity. Burley tobaccos are light to dark brown in color, high in oil, and low in sugar. Burley tobaccos are air-cured in barns. Major Burley growing countries are Argentina, Brazil, Italy, Malawi, and the U.S. Maryland tobaccos are extremely fluffy, have good burning properties, low nicotine and a neutral aroma. Major Maryland growing countries include the U.S. and Italy. In one aspect, modified tobacco plants or seeds provided herein are in a Burley tobacco background selected from the group consisting of Clay 402, Clay 403, Clay 502, Ky 14, Ky 907, Ky 910, Ky 8959, NC 2, NC 3, NC 4, NC 5, NC 2000, TN 86, TN 90, TN 97, R 610, R 630, R 711, R 712, NCBH 129, HB4488PLC, PD 7319LC, Bu 21×Ky 10, HB04P, Ky 14×L 8, Kt 200, Newton 98, Pedigo 561, Pf561 and Va 509. In further aspects, modified tobacco plants, seeds, hybrids, varieties, or lines provided herein are in any Burley background selected from the group consisting of TN 90, KT 209, KT 206, KT212, and HB 4488. In another aspect, modified tobacco plants or seeds provided herein are in a Maryland tobacco background selected from the group consisting of Md 10, Md 40, Md 201, Md 609, Md 872 and Md 341.

Dark air-cured tobaccos are distinguished from other types primarily by its curing process which gives dark air-cured tobacco its medium- to dark-brown color and distinct aroma. Dark air-cured tobaccos are mainly used in the production of chewing tobacco and snuff. In one aspect, modified tobacco plants or seeds provided herein are in a dark air-cured tobacco background selected from the group consisting of Sumatra, Jatim, Dominican Cubano, Besuki, One sucker, Green River, Virginia sun-cured, and Paraguan Passado.

Dark fire-cured tobaccos are generally cured with low-burning wood fires on the floors of closed curing barns. Dark fire-cured tobaccos are used for making pipe blends, cigarettes, chewing tobacco, snuff and strong-tasting cigars. Major growing regions for dark fire-cured tobaccos are Tennessee, Kentucky, and Virginia, USA. In one aspect, modified tobacco plants or seeds provided herein are in a dark fire-cured tobacco background selected from the group consisting of Narrow Leaf Madole, Improved Madole, Tom Rosson Madole, Newton's VH Madole, Little Crittenden, Green Wood, Little Wood, Small Stalk Black Mammoth, DT 508, DT 518, DT 592, KY 171, DF 911, DF 485, TN D94, TN D950, VA 309, and VA 359.

Oriental tobaccos are also referred to as Greek, aroma and Turkish tobaccos due to the fact that they are typically grown in eastern Mediterranean regions such as Turkey, Greece, Bulgaria, Macedonia, Syria, Lebanon, Italy, and Romania. The small plant and leaf size, characteristic of today's Oriental varieties, as well as its unique aroma properties are a result of the plant's adaptation to the poor soil and stressful climatic conditions in which it develop over many past centuries. In one aspect, modified tobacco plants or seeds provided herein are in an Oriental tobacco background selected from the group consisting of Izmir, Katerini, Samsun, Basma and Krumovgrad, Trabzon, Thesalian, Tasova, Sinop, Izmit, Hendek, Edirne, Semdinli, Adiyanman, Yayladag, Iskenderun, Duzce, Macedonian, Mavra, Prilep, Bafra, Bursa, Bucak, Bitlis, Balikesir, and any variety essentially derived from any one of the foregoing varieties.

In one aspect, modified tobacco plants, seeds, hybrids, varieties, or lines provided herein are essentially derived from or in the genetic background of BU 64, CC 101, CC 200, CC 13, CC 27, CC 33, CC 35, CC 37, CC 65, CC 67, CC 301, CC 400, CC 500, CC 600, CC 700, CC 800, CC 900, CC 1063, Coker 176, Coker 319, Coker 371 Gold, Coker 48, CU 263, DF911, Galpão, GL 26H, GL 338, GL 350, GL 395, GL 600, GL 737, GL 939, GL 973, GF 157, GF 318, RJR 901, HB 04P, K 149, K 326, K 346, K 358, K394, K 399, K 730, NC 196, NC 37NF, NC 471, NC 55, NC 92, NC2326, NC 95, NC 925, PVH 1118, PVH 1452, PVH 2110, PVH 2254, PVH 2275, VA 116, VA 119, KDH 959, KT 200, KT204LC, KY 10, KY 14, KY 160, KY 17, KY 171, KY 907, KY 907LC, KTY14×L8 LC, Little Crittenden, McNair 373, McNair 944, male sterile KY 14×L8, Narrow Leaf Madole, MS KY171, Narrow Leaf Madole (phph), MS Narrow Leaf Madole, MS TND950, PD 7302LC, PD 7305LC, PD 7309LC, PD 7312LC, PD 7318LC, PD 7319LC, MSTKS 2002, TKF 2002, TKF 6400, TKF 4028, TKF 4024, KT206LC, KT209LC, KT210LC, KT212LC, NC 100, NC 102, NC 2000, NC 291, NC 297, NC 299, NC 3, NC 4, NC 5, NC 6, NC7, NC 606, NC 71, NC 72, NC 810, NC BH 129, NC 2002, Neal Smith Madole, OXFORD 207, ‘Perique’, PVH03, PVH09, PVH19, PVH50, PVH51, R 610, R 630, R 7-11, R 7-12, RG 17, RG 81, RG H51, RGH 4, RGH 51, RS 1410, Speight 168, Speight 172, Speight 179, Speight 210, Speight 220, Speight 225, Speight 227, Speight 234, Speight G-28, Speight G-70, Speight H-6, Speight H20, Speight NF3, TI 1406, TI 1269, TN 86, TN86LC, TN 90, TN90LC, TN 97, TN97LC, TN D94, TN D950, a TR (Tom Rosson) Madole, VA 309, VA 359, or any commercial tobacco variety according to standard tobacco breeding techniques known in the art.

All foregoing mentioned specific varieties of dark air-cured, Burley, Maryland, dark fire-cured, or Oriental type are only listed for exemplary purposes. Any additional dark air-cured, Burley, Maryland, dark fire-cured, Oriental varieties are also contemplated in the present application.

Also provided herein are populations of tobacco plants described herein. In one aspect, a population of tobacco plants provided herein has a planting density of between 5,000 and 8000, between 5,000 and 7,600, between 5,000 and 7,200, between 5,000 and 6,800, between 5,000 and 6,400, between 5,000 and 6,000, between 5,000 and 5,600, between 5,000 and 5,200, between 5,200 and 8,000, between 5,600 and 8,000, between 6,000 and 8,000, between 6,400 and 8,000, between 6,800 and 8,000, between 7,200 and 8,000, or between 7,600 and 8,000 plants per acre.

Also provided herein are containers of seeds from tobacco plants described herein. A container of tobacco seeds of the present disclosure may contain any number, weight, or volume of seeds. For example, a container can contain at least or greater than 10 seeds; at least or greater than 25 seeds; at least or greater than 50 seeds; at least, or greater than, 100 seeds; at least, or greater than, 200 seeds; at least, or greater than, 300 seeds; at least, or greater than, 400 seeds; at least, or greater than, 500 seeds; at least, or greater than, 600 seeds; at least, or greater than, 700 seeds; at least, or greater than, 800 seeds; at least, or greater than, 900 seeds; at least, or greater than, 1000 seeds; at least, or greater than, 1500 seeds; at least, or greater than, 2000 seeds; at least, or greater than, 2500 seeds; at least, or greater than, 3000 seeds; at least, or greater than, 3500 seeds; at least, or greater than, 4000 seeds; or at least, or greater than 5000 seeds. Alternatively, the container can contain at least, or greater than, 1 ounce of seeds; at least, or greater than, 5 grams of seeds; at least, or greater than, 10 grams of seeds; at least, or greater than, 30 grams of seeds; at least, or greater than, 50 grams of seeds; at least, or greater than, 100 grams of seeds; at least, or greater than, 500 grams of seeds; at least, or greater than, 1 kilogram of seeds; at least, or greater than 1.5 kilograms of seeds; at least, or greater than 2 kilograms of seeds; at least, or greater than, 5 kilograms of seeds; or at least, or greater than, 10 kilograms of seeds. Containers of tobacco seeds may be any container available in the art. By way of non-limiting example, a container may be a box, a bag, a packet, a pouch, a tape roll, a tube, or a bottle.

In one aspect, present disclosure provides cured leaf from a modified tobacco plant comprising a reduced level of one or more TSNAs. In one aspect, reduced one or more TSNAs are selected from the group consisting of N′-nitrosonornicotine (NNN), 4-methylnitrosoamino-l-(3-pyridyl)-1-butanone (NNK), N′-nitrosoanatabine (NAT) N′-nitrosoanabasine (NAB), and any combination thereof. In one aspect, the level of total TSNAs or an individual TSNA is measured based on a freeze-dried cured leaf sample using liquid chromatograph with tandem mass spectrometry (LC/MS/MS).

In one aspect, present disclosure provides cured leaf from a modified tobacco plant comprising a reduced level of one or more alkaloids. In one aspect, reduced one or more alkaloids are selected from the group consisting of nicotine, nornicotine, anabasine, anatabine.

The present disclosure also provides methods for breeding tobacco lines, cultivars, or varieties comprising enhanced nitrogen utilization efficiency. Breeding can be carried out via any known procedures. DNA fingerprinting, SNP mapping, haplotype mapping or similar technologies may be used in a marker-assisted selection (MAS) breeding program to transfer or breed a desirable trait or allele into a tobacco plant. For example, a breeder can create segregating populations in an F₂ or backcross generation using F₁ hybrid plants provided herein or further crossing the F₁ hybrid plants with other donor plants with an agronomically desirable genotype. Plants in the F₂ or backcross generations can be screened for a desired agronomic trait or a desirable chemical profile using one of the techniques known in the art or listed herein. Depending on the expected inheritance pattern or the MAS technology used, self-pollination of selected plants before each cycle of backcrossing to aid identification of the desired individual plants can be performed. Backcrossing or other breeding procedure can be repeated until the desired phenotype of the recurrent parent is recovered. In one aspect, a recurrent parent in the present disclosure can be a flue-cured variety, a Burley variety, a dark air-cured variety, a dark fire-cured variety, or an Oriental variety. In another aspect, a recurrent parent can be a modified tobacco plant, line, or variety. In one aspect, a recurrent parent provided herein is TN90. In another aspect, a recurrent parent provided herein is MD609. Other breeding techniques can be found, for example, in Wernsman, E. A., and Rufty, R. C. 1987. Chapter Seventeen. Tobacco. Pages 669-698 In: Cultivar Development. Crop Species. W. H. Fehr (ed.), MacMillan Publishing Go., Inc., New York, N.Y., incorporated herein by reference in their entirety.

Results of a plant breeding program using modified tobacco plants described herein includes useful lines, cultivars, varieties, progeny, inbreds, and hybrids of the present disclosure. As used herein, the term “variety” refers to a population of plants that share constant characteristics which separate them from other plants of the same species. A variety is often, although not always, sold commercially. While possessing one or more distinctive traits, a variety is further characterized by a very small overall variation between individuals within that variety. A “pure line” variety may be created by several generations of self-pollination and selection, or vegetative propagation from a single parent using tissue or cell culture techniques. A variety can be essentially derived from another line or variety. As defined by the International Convention for the Protection of New Varieties of Plants (Dec. 2, 1961, as revised at Geneva on Nov. 10, 1972; on Oct. 23, 1978; and on Mar. 19, 1991), a variety is “essentially derived” from an initial variety if: a) it is predominantly derived from the initial variety, or from a variety that is predominantly derived from the initial variety, while retaining the expression of the essential characteristics that result from the genotype or combination of genotypes of the initial variety; b) it is clearly distinguishable from the initial variety; and c) except for the differences which result from the act of derivation, it conforms to the initial variety in the expression of the essential characteristics that result from the genotype or combination of genotypes of the initial variety. Essentially derived varieties can be obtained, for example, by the selection of a natural or induced mutant, a somaclonal variant, a variant individual from plants of the initial variety, backcrossing, or transformation. A first tobacco variety and a second tobacco variety from which the first variety is essentially derived, are considered as having essentially identical genetic background. A “line” as distinguished from a variety most often denotes a group of plants used non-commercially, for example in plant research. A line typically displays little overall variation between individuals for one or more traits of interest, although there may be some variation between individuals for other traits.

In one aspect, the present disclosure provides a method of producing a tobacco plant comprising crossing at least one tobacco plant of a first tobacco variety with at least one tobacco plant of a second tobacco variety, where the at least one tobacco plant of the first tobacco variety exhibits enhanced nitrogen utilization efficiency compared to a control tobacco plant of the same variety grown under comparable conditions; and selecting for progeny tobacco plants that exhibit enhanced nitrogen utilization efficiency compared to a control tobacco plant of the same cross grown under comparable conditions. In one aspect, a first tobacco variety provided herein comprises modified tobacco plants. In another aspect, a second tobacco variety provided herein comprises modified tobacco plants. In one aspect, a first or second tobacco variety is male sterile. In another aspect, a first or second tobacco variety is cytoplasmically male sterile. In another aspect, a first or second tobacco variety is female sterile. In one aspect, a first or second tobacco variety is an elite variety. In another aspect, a first or second tobacco variety is a hybrid.

In one aspect, the present disclosure provides a method of introgressing one or more transgenes into a tobacco variety, the method comprising: (a) crossing a first tobacco variety comprising one or more transgenes with a second tobacco variety without the one or more transgenes to produce one or more progeny tobacco plants; (b) genotyping the one or more progeny tobacco plants for the one or more transgenes; and (c) selecting a progeny tobacco plant comprising the one or more transgenes. In another aspect, these methods further comprise backcrossing the selected progeny tobacco plant with the second tobacco variety. In further aspects, these methods further comprise: (d) crossing the selected progeny plant with itself or with the second tobacco variety to produce one or more further progeny tobacco plants; and (e) selecting a further progeny tobacco plant comprising the one or more transgenes. In one aspect, the second tobacco variety is an elite variety.

In one aspect, the present disclosure provides a method of introgressing one or more mutations into a tobacco variety, the method comprising: (a) crossing a first tobacco variety comprising one or more mutations with a second tobacco variety without the one or more mutations to produce one or more progeny tobacco plants; (b) genotyping the one or more progeny tobacco plants for the one or more mutations; and (c) selecting a progeny tobacco plant comprising the one or more mutations. In another aspect, these methods further comprise backcrossing the selected progeny tobacco plant with the second tobacco variety. In further aspects, these methods further comprise: (d) crossing the selected progeny plant with itself or with the second tobacco variety to produce one or more further progeny tobacco plants; and (e) selecting a further progeny tobacco plant comprising the one or more mutations. In one aspect, the second tobacco variety is an elite variety.

In one aspect, the present disclosure provides a method of growing a population of modified tobacco plants comprising enhanced nitrogen utilization efficiency, where the method comprises planting a population of tobacco seeds comprising one or more mutations, one or more transgenes, or both, where the one or more modified tobacco plants exhibit enhanced nitrogen utilization efficiency compared to control tobacco plants of the same variety when grown under comparable conditions.

In one aspect, this disclosure provides a method for manufacturing a modified seed, comprising introducing a recombinant DNA construct provided herein into a plant cell; screening a population of plant cells for the recombinant DNA construct; selecting one or more plant cells from the population; generating one or more modified plants from the one or more plant cells; and collecting one or more modified seeds from the one or more modified plants.

As used herein, “locus” is a chromosome region where a polymorphic nucleic acid, trait determinant, gene, or marker is located. The loci of this disclosure comprise one or more polymorphisms in a population; e.g., alternative alleles are present in some individuals. As used herein, “allele” refers to an alternative nucleic acid sequence at a particular locus. The length of an allele can be as small as 1 nucleotide base, but is typically larger. For example, a first allele can occur on one chromosome, while a second allele occurs on a second homologous chromosome, e.g., as occurs for different chromosomes of a heterozygous individual, or between different homozygous or heterozygous individuals in a population. As used herein, a chromosome in a diploid plant is “hemizygous” when only one copy of a locus is present. For example, an inserted transgene is hemizygous when it only inserts into one sister chromosome (e.g., the second sister chromosome does not contain the inserted transgene).

In one aspect, a modified plant, seed, plant component, plant cell, or plant genome is homozygous for a transgene provided herein. In another aspect, a modified plant, seed, plant component, plant cell, or plant genome is heterozygous for a transgene provided herein. In one aspect, a modified plant, seed, plant component, plant cell, or plant genome is hemizygous for a transgene provided herein. In one aspect, a modified plant, seed, plant component, plant cell, or plant genome is homozygous for a mutation provided herein. In another aspect, a modified plant, seed, plant component, plant cell, or plant genome is heterozygous for a mutation provided herein. In one aspect, a modified plant, seed, plant component, plant cell, or plant genome is hemizygous for a mutation provided herein.

As used herein, “introgression” or “introgress” refers to the transmission of a desired allele of a genetic locus from one genetic background to another.

As used herein, “crossed” or “cross” means to produce progeny via fertilization (e.g. cells, seeds or plants) and includes crosses between different plants (sexual) and self-fertilization (selfing).

As used herein, “backcross” and “backcrossing” refer to the process whereby a progeny plant is repeatedly crossed back to one of its parents. In a backcrossing scheme, the “donor” parent refers to the parental plant with the desired gene or locus to be introgressed. The “recipient” parent (used one or more times) or “recurrent” parent (used two or more times) refers to the parental plant into which the gene or locus is being introgressed. The initial cross gives rise to the F₁ generation. The term “BC1” refers to the second use of the recurrent parent, “BC2” refers to the third use of the recurrent parent, and so on. In one aspect, a backcross is performed repeatedly, with a progeny individual of each successive backcross generation being itself backcrossed to the same parental genotype.

As used herein, “elite variety” means any variety that has resulted from breeding and selection for superior agronomic performance.

As used herein, “selecting” or “selection” in the context of breeding refer to the act of picking or choosing desired individuals, normally from a population, based on certain pre-determined criteria.

In one aspect, tobacco plants provided herein are hybrid plants. Hybrids can be produced by preventing self-pollination of female parent plants (e.g., seed parents) of a first variety, permitting pollen from male parent plants of a second variety to fertilize the female parent plants, and allowing F₁ hybrid seeds to form on the female plants. Self-pollination of female plants can be prevented by emasculating the flowers at an early stage of flower development. Alternatively, pollen formation can be prevented on the female parent plants using a form of male sterility. For example, male sterility can be produced by male sterility (MS), or transgenic male sterility where a transgene inhibits microsporogenesis and/or pollen formation, or self-incompatibility. Female parent plants containing MS are particularly useful. In aspects in which the female parent plants are MS, pollen may be harvested from male fertile plants and applied manually to the stigmas of MS female parent plants, and the resulting F₁ seed is harvested. Additionally, female sterile plants can also be used to prevent self-fertilization.

Plants can be used to form single-cross tobacco F₁ hybrids. Pollen from a male parent plant is manually transferred to an emasculated female parent plant or a female parent plant that is male sterile to form F₁ seed. Alternatively, three-way crosses can be carried out where a single-cross F₁ hybrid is used as a female parent and is crossed with a different male parent. As another alternative, double-cross hybrids can be created where the F₁ progeny of two different single-crosses are themselves crossed. Self-incompatibility can be used to particular advantage to prevent self-pollination of female parents when forming a double-cross hybrid.

In one aspect, a tobacco variety provided herein is male sterile. In another aspect, a tobacco variety provided herein is cytoplasmic male sterile (CMS). Male sterile tobacco plants may be produced by any method known in the art. Methods of producing male sterile tobacco are described in Wernsman, E. A., and Rufty, R. C. 1987. Chapter Seventeen. Tobacco. Pages 669-698 In: Cultivar Development. Crop Species. W. H. Fehr (ed.), MacMillan Publishing Go., Inc., New York, N.Y. 761 pp. In another aspect, a tobacco variety provided herein is female sterile. As a non-limiting example, female sterile plants can be made by mutating the STIG1 gene. See, for example, Goldman et al. 1994, EMBO Journal 13:2976-2984.

In one aspect, the present disclosure provides for, and includes, a method of determining the NUE of a tobacco line comprising obtaining at least one metabolite from a tobacco plant of a tobacco line, determining the amount of the at least one obtained metabolites, and determining the NUE of the tobacco line based on the amount of the at least one metabolite determined. In a further aspect, the at least one metabolite is obtained from a plant tissue selected from the group consisting of root tissue, leaf tissue, floral tissue, meristem tissue, and stem tissue. In a further aspect of this method, at least two metabolites are obtained. In a further aspect of this method, at least three metabolites are obtained. In a further aspect of this method, at least four metabolites are obtained. In a further aspect of this method, at least five metabolites are obtained. In a further aspect of this method, at least six metabolites are obtained. In a further aspect of this method, at least seven metabolites are obtained. In a further aspect of this method, at least eight metabolites are obtained. In a further aspect of this method, at least nine metabolites are obtained. In a further aspect of this method, at least ten metabolites are obtained. In a further aspect of this method, the amount of at least two metabolites is determined. In a further aspect of this method, the amount of at least three metabolites is determined. In a further aspect of this method, the amount of at least four metabolites is determined. In a further aspect of this method, the amount of at least five metabolites is determined. In a further aspect of this method, the amount of at least six metabolites is determined. In a further aspect of this method, the amount of at least seven metabolites is determined. In a further aspect of this method, the amount of at least eight metabolites is determined. In a further aspect of this method, the amount of at least nine metabolites is determined. In a further aspect of this method, the amount of at least ten metabolites is determined.

In another aspect of a method provided herein, the amount of a metabolite selected from the group consisting of X-2357, N-acetylmuramate, X-23319, X-23852, X-23330, alpha-ketoglutarate, X-21756, 4-hydroxy-2-oxoglutaric acid, D-23937, X-23937, X-23916, 1-methyladenine, 4-guanidinobutanoate, syringaldehyde, thiamin, p-hydroxybenzaldehyde, X-23453, X-11429, X-21796, N′-methylnicotinamide, cotinine, X-23389, N-acetylarginine, X-23366, N-acetylphenylalanine, naringenin, X-23454, X-23580, and X-23852 is determined.

In another aspect of a method provided herein, a tobacco plant with enhanced NUE comprises enhanced NUE as compared to a tobacco plant that comprises a lower amount of at least one metabolite in at least one tissue. In a further aspect, a tobacco plant with enhanced NUE comprises a lower amount of at least two metabolites in at least one tissue. In a further aspect, a tobacco plant with enhanced NUE comprises a lower amount of at least three metabolites in at least one tissue. In a further aspect, a tobacco plant with enhanced NUE comprises a lower amount of at least four metabolites in at least one tissue. In a further aspect, a tobacco plant with enhanced NUE comprises a lower amount of at least five metabolites in at least one tissue. In a further aspect, a tobacco plant with enhanced NUE comprises a lower amount of at least one metabolite in two tissues. In a further aspect, a tobacco plant with enhanced NUE comprises a lower amount of at least two metabolites in at least two tissues. In a further aspect, a tobacco plant with enhanced NUE comprises a lower amount of at least three metabolites in at least two tissues. In a further aspect, a tobacco plant with enhanced NUE comprises a lower amount of at least four metabolites in at least two tissues. In a further aspect, a tobacco plant with enhanced NUE comprises a lower amount of at least five metabolites in at least two tissues. In a further aspect, a tobacco plant with enhanced NUE comprises a lower amount of at least one metabolite in three tissues. In a further aspect, a tobacco plant with enhanced NUE comprises a lower amount of at least two metabolites in at least three tissues. In a further aspect, a tobacco plant with enhanced NUE comprises a lower amount of at least three metabolites in at least three tissues. In a further aspect, a tobacco plant with enhanced NUE comprises a lower amount of at least four metabolites in at least three tissues. In a further aspect, a tobacco plant with enhanced NUE comprises a lower amount of at least five metabolites in at least three tissues. In a further aspect, a tobacco plant with enhanced NUE comprises a lower amount of at least one metabolite in four tissues. In a further aspect, a tobacco plant with enhanced NUE comprises a lower amount of at least two metabolites in at least four tissues. In a further aspect, a tobacco plant with enhanced NUE comprises a lower amount of at least three metabolites in at least four tissues. In a further aspect, a tobacco plant with enhanced NUE comprises a lower amount of at least four metabolites in at least four tissues. In a further aspect, a tobacco plant with enhanced NUE comprises a lower amount of at least five metabolites in at least four tissues. In a further aspect, a tobacco plant with enhanced NUE comprises a lower amount of at least one metabolite in five tissues. In a further aspect, a tobacco plant with enhanced NUE comprises a lower amount of at least two metabolites in at least five tissues. In a further aspect, a tobacco plant with enhanced NUE comprises a lower amount of at least three metabolites in at least five tissues. In a further aspect, a tobacco plant with enhanced NUE comprises a lower amount of at least four metabolites in at least five tissues. In a further aspect, a tobacco plant with enhanced NUE comprises a lower amount of at least five metabolites in at least five tissues.

In another aspect of a method provided herein, a tobacco plant with enhanced NUE comprises enhanced NUE as compared to a tobacco line that comprises a higher amount of at least one metabolite in at least one tissue. In a further aspect, a tobacco plant with enhanced NUE comprises a higher amount of at least two metabolites in at least one tissue. In a further aspect, a tobacco plant with enhanced NUE comprises a higher amount of at least three metabolites in at least one tissue. In a further aspect, a tobacco plant with enhanced NUE comprises a higher amount of at least four metabolites in at least one tissue. In a further aspect, a tobacco plant with enhanced NUE comprises a higher amount of at least five metabolites in at least one tissue. In a further aspect, a tobacco plant with enhanced NUE comprises a higher amount of at least one metabolite in two tissues. In a further aspect, a tobacco plant with enhanced NUE comprises a higher amount of at least two metabolites in at least two tissues. In a further aspect, a tobacco plant with enhanced NUE comprises a higher amount of at least three metabolites in at least two tissues. In a further aspect, a tobacco plant with enhanced NUE comprises a higher amount of at least four metabolites in at least two tissues. In a further aspect, a tobacco plant with enhanced NUE comprises a higher amount of at least five metabolites in at least two tissues. In a further aspect, a tobacco plant with enhanced NUE comprises a higher amount of at least one metabolite in three tissues. In a further aspect, a tobacco plant with enhanced NUE comprises a higher amount of at least two metabolites in at least three tissues. In a further aspect, a tobacco plant with enhanced NUE comprises a higher amount of at least three metabolites in at least three tissues. In a further aspect, a tobacco plant with enhanced NUE comprises a higher amount of at least four metabolites in at least three tissues. In a further aspect, a tobacco plant with enhanced NUE comprises a higher amount of at least five metabolites in at least three tissues. In a further aspect, a tobacco plant with enhanced NUE comprises a higher amount of at least one metabolite in four tissues. In a further aspect, a tobacco plant with enhanced NUE comprises a higher amount of at least two metabolites in at least four tissues. In a further aspect, a tobacco plant with enhanced NUE comprises a higher amount of at least three metabolites in at least four tissues. In a further aspect, a tobacco plant with enhanced NUE comprises a higher amount of at least four metabolites in at least four tissues. In a further aspect, a tobacco plant with enhanced NUE comprises a higher amount of at least five metabolites in at least four tissues. In a further aspect, a tobacco plant with enhanced NUE comprises a higher amount of at least one metabolite in five tissues. In a further aspect, a tobacco plant with enhanced NUE comprises a higher amount of at least two metabolites in at least five tissues. In a further aspect, a tobacco plant with enhanced NUE comprises a higher amount of at least three metabolites in at least five tissues. In a further aspect, a tobacco plant with enhanced NUE comprises a higher amount of at least four metabolites in at least five tissues. In a further aspect, a tobacco plant with enhanced NUE comprises a higher amount of at least five metabolites in at least five tissues.

In another aspect of a method provided herein, a tobacco plant with enhanced NUE comprises enhanced NUE as compared to a tobacco line that comprises an equal amount of at least one metabolite in at least one tissue. In a further aspect, a tobacco plant with enhanced NUE comprises an equal amount of at least two metabolites in at least one tissue. In a further aspect, a tobacco plant with enhanced NUE comprises an equal amount of at least three metabolites in at least one tissue. In a further aspect, a tobacco plant with enhanced NUE comprises an equal amount of at least four metabolites in at least one tissue. In a further aspect, a tobacco plant with enhanced NUE comprises an equal amount of at least five metabolites in at least one tissue. In a further aspect, a tobacco plant with enhanced NUE comprises an equal amount of at least one metabolite in two tissues. In a further aspect, a tobacco plant with enhanced NUE comprises an equal amount of at least two metabolites in at least two tissues. In a further aspect, a tobacco plant with enhanced NUE comprises an equal amount of at least three metabolites in at least two tissues. In a further aspect, a tobacco plant with enhanced NUE comprises an equal amount of at least four metabolites in at least two tissues. In a further aspect, a tobacco plant with enhanced NUE comprises an equal amount of at least five metabolites in at least two tissues. In a further aspect, a tobacco plant with enhanced NUE comprises an equal amount of at least one metabolite in three tissues. In a further aspect, a tobacco plant with enhanced NUE comprises an equal amount of at least two metabolites in at least three tissues. In a further aspect, a tobacco plant with enhanced NUE comprises an equal amount of at least three metabolites in at least three tissues. In a further aspect, a tobacco plant with enhanced NUE comprises an equal amount of at least four metabolites in at least three tissues. In a further aspect, a tobacco plant with enhanced NUE comprises an equal amount of at least five metabolites in at least three tissues. In a further aspect, a tobacco plant with enhanced NUE comprises an equal amount of at least one metabolite in four tissues. In a further aspect, a tobacco plant with enhanced NUE comprises an equal amount of at least two metabolites in at least four tissues. In a further aspect, a tobacco plant with enhanced NUE comprises an equal amount of at least three metabolites in at least four tissues. In a further aspect, a tobacco plant with enhanced NUE comprises an equal amount of at least four metabolites in at least four tissues. In a further aspect, a tobacco plant with enhanced NUE comprises an equal amount of at least five metabolites in at least four tissues. In a further aspect, a tobacco plant with enhanced NUE comprises an equal amount of at least one metabolite in five tissues. In a further aspect, a tobacco plant with enhanced NUE comprises an equal amount of at least two metabolites in at least five tissues. In a further aspect, a tobacco plant with enhanced NUE comprises an equal amount of at least three metabolites in at least five tissues. In a further aspect, a tobacco plant with enhanced NUE comprises an equal amount of at least four metabolites in at least five tissues. In a further aspect, a tobacco plant with enhanced NUE comprises an equal amount of at least five metabolites in at least five tissues.

In another aspect of a method provided herein, a tobacco plant with enhanced NUE comprises decreased NUE as compared to a tobacco line that comprises a lower amount of at least one metabolite in at least one tissue. In a further aspect, a tobacco plant with enhanced NUE comprises a lower amount of at least two metabolites in at least one tissue. In a further aspect, a tobacco plant with enhanced NUE comprises a lower amount of at least three metabolites in at least one tissue. In a further aspect, a tobacco plant with enhanced NUE comprises a lower amount of at least four metabolites in at least one tissue. In a further aspect, a tobacco plant with enhanced NUE comprises a lower amount of at least five metabolites in at least one tissue. In a further aspect, a tobacco plant with enhanced NUE comprises a lower amount of at least one metabolite in two tissues. In a further aspect, a tobacco plant with enhanced NUE comprises a lower amount of at least two metabolites in at least two tissues. In a further aspect, a tobacco plant with enhanced NUE comprises a lower amount of at least three metabolites in at least two tissues. In a further aspect, a tobacco plant with enhanced NUE comprises a lower amount of at least four metabolites in at least two tissues. In a further aspect, a tobacco plant with enhanced NUE comprises a lower amount of at least five metabolites in at least two tissues. In a further aspect, a tobacco plant with enhanced NUE comprises a lower amount of at least one metabolite in three tissues. In a further aspect, a tobacco plant with enhanced NUE comprises a lower amount of at least two metabolites in at least three tissues. In a further aspect, a tobacco plant with enhanced NUE comprises a lower amount of at least three metabolites in at least three tissues. In a further aspect, a tobacco plant with enhanced NUE comprises a lower amount of at least four metabolites in at least three tissues. In a further aspect, a tobacco plant with enhanced NUE comprises a lower amount of at least five metabolites in at least three tissues. In a further aspect, a tobacco plant with enhanced NUE comprises a lower amount of at least one metabolite in four tissues. In a further aspect, a tobacco plant with enhanced NUE comprises a lower amount of at least two metabolites in at least four tissues. In a further aspect, a tobacco plant with enhanced NUE comprises a lower amount of at least three metabolites in at least four tissues. In a further aspect, a tobacco plant with enhanced NUE comprises a lower amount of at least four metabolites in at least four tissues. In a further aspect, a tobacco plant with enhanced NUE comprises a lower amount of at least five metabolites in at least four tissues. In a further aspect, a tobacco plant with enhanced NUE comprises a lower amount of at least one metabolite in five tissues. In a further aspect, a tobacco plant with enhanced NUE comprises a lower amount of at least two metabolites in at least five tissues. In a further aspect, a tobacco plant with enhanced NUE comprises a lower amount of at least three metabolites in at least five tissues. In a further aspect, a tobacco plant with enhanced NUE comprises a lower amount of at least four metabolites in at least five tissues. In a further aspect, a tobacco plant with enhanced NUE comprises a lower amount of at least five metabolites in at least five tissues.

In another aspect of a method provided herein, a tobacco plant with enhanced NUE comprises decreased NUE as compared to a tobacco line that comprises a higher amount of at least one metabolite in at least one tissue. In a further aspect, a tobacco plant with enhanced NUE comprises a higher amount of at least two metabolites in at least one tissue. In a further aspect, a tobacco plant with enhanced NUE comprises a higher amount of at least three metabolites in at least one tissue. In a further aspect, a tobacco plant with enhanced NUE comprises a higher amount of at least four metabolites in at least one tissue. In a further aspect, a tobacco plant with enhanced NUE comprises a higher amount of at least five metabolites in at least one tissue. In a further aspect, a tobacco plant with enhanced NUE comprises a higher amount of at least one metabolite in two tissues. In a further aspect, a tobacco plant with enhanced NUE comprises a higher amount of at least two metabolites in at least two tissues. In a further aspect, a tobacco plant with enhanced NUE comprises a higher amount of at least three metabolites in at least two tissues. In a further aspect, a tobacco plant with enhanced NUE comprises a higher amount of at least four metabolites in at least two tissues. In a further aspect, a tobacco plant with enhanced NUE comprises a higher amount of at least five metabolites in at least two tissues. In a further aspect, a tobacco plant with enhanced NUE comprises a higher amount of at least one metabolite in three tissues. In a further aspect, a tobacco plant with enhanced NUE comprises a higher amount of at least two metabolites in at least three tissues. In a further aspect, a tobacco plant with enhanced NUE comprises a higher amount of at least three metabolites in at least three tissues. In a further aspect, a tobacco plant with enhanced NUE comprises a higher amount of at least four metabolites in at least three tissues. In a further aspect, a tobacco plant with enhanced NUE comprises a higher amount of at least five metabolites in at least three tissues. In a further aspect, a tobacco plant with enhanced NUE comprises a higher amount of at least one metabolite in four tissues. In a further aspect, a tobacco plant with enhanced NUE comprises a higher amount of at least two metabolites in at least four tissues. In a further aspect, a tobacco plant with enhanced NUE comprises a higher amount of at least three metabolites in at least four tissues. In a further aspect, a tobacco plant with enhanced NUE comprises a higher amount of at least four metabolites in at least four tissues. In a further aspect, a tobacco plant with enhanced NUE comprises a higher amount of at least five metabolites in at least four tissues. In a further aspect, a tobacco plant with enhanced NUE comprises a higher amount of at least one metabolite in five tissues. In a further aspect, a tobacco plant with enhanced NUE comprises a higher amount of at least two metabolites in at least five tissues. In a further aspect, a tobacco plant with enhanced NUE comprises a higher amount of at least three metabolites in at least five tissues. In a further aspect, a tobacco plant with enhanced NUE comprises a higher amount of at least four metabolites in at least five tissues. In a further aspect, a tobacco plant with enhanced NUE comprises a higher amount of at least five metabolites in at least five tissues.

In another aspect of a method provided herein, a tobacco plant with enhanced NUE comprises decreased NUE as compared to a tobacco line that comprises an equal amount of at least one metabolite in at least one tissue. In a further aspect, a tobacco plant with enhanced NUE comprises an equal amount of at least two metabolites in at least one tissue. In a further aspect, a tobacco plant with enhanced NUE comprises an equal amount of at least three metabolites in at least one tissue. In a further aspect, a tobacco plant with enhanced NUE comprises an equal amount of at least four metabolites in at least one tissue. In a further aspect, a tobacco plant with enhanced NUE comprises an equal amount of at least five metabolites in at least one tissue. In a further aspect, a tobacco plant with enhanced NUE comprises an equal amount of at least one metabolite in two tissues. In a further aspect, a tobacco plant with enhanced NUE comprises an equal amount of at least two metabolites in at least two tissues. In a further aspect, a tobacco plant with enhanced NUE comprises an equal amount of at least three metabolites in at least two tissues. In a further aspect, a tobacco plant with enhanced NUE comprises an equal amount of at least four metabolites in at least two tissues. In a further aspect, a tobacco plant with enhanced NUE comprises an equal amount of at least five metabolites in at least two tissues. In a further aspect, a tobacco plant with enhanced NUE comprises an equal amount of at least one metabolite in three tissues. In a further aspect, a tobacco plant with enhanced NUE comprises an equal amount of at least two metabolites in at least three tissues. In a further aspect, a tobacco plant with enhanced NUE comprises an equal amount of at least three metabolites in at least three tissues. In a further aspect, a tobacco plant with enhanced NUE comprises an equal amount of at least four metabolites in at least three tissues. In a further aspect, a tobacco plant with enhanced NUE comprises an equal amount of at least five metabolites in at least three tissues. In a further aspect, a tobacco plant with enhanced NUE comprises an equal amount of at least one metabolite in four tissues. In a further aspect, a tobacco plant with enhanced NUE comprises an equal amount of at least two metabolites in at least four tissues. In a further aspect, a tobacco plant with enhanced NUE comprises an equal amount of at least three metabolites in at least four tissues. In a further aspect, a tobacco plant with enhanced NUE comprises an equal amount of at least four metabolites in at least four tissues. In a further aspect, a tobacco plant with enhanced NUE comprises an equal amount of at least five metabolites in at least four tissues. In a further aspect, a tobacco plant with enhanced NUE comprises an equal amount of at least one metabolite in five tissues. In a further aspect, a tobacco plant with enhanced NUE comprises an equal amount of at least two metabolites in at least five tissues. In a further aspect, a tobacco plant with enhanced NUE comprises an equal amount of at least three metabolites in at least five tissues. In a further aspect, a tobacco plant with enhanced NUE comprises an equal amount of at least four metabolites in at least five tissues. In a further aspect, a tobacco plant with enhanced NUE comprises an equal amount of at least five metabolites in at least five tissues.

In another aspect, a method provided herein comprises determining the amount of a metabolite using a method selected from the group consisting of liquid chromatography/mass spectrometry (LC/MS), high-performance liquid chromatography (HPLC), ultra HPLC (UHPLC), mass spectrometry (MS), tandem mass spectrometry (MS/MS), matrix assisted laser desorption/ionization mass spectrometry (MALDI-MS), X-ray fluorescence spectrometry (XRF), ion chromatography (IC), gas chromatography (GC), gas chromatography/mass spectrometry (GC/MS), capillary electrophoresis/mass spectrometry (CE-MS), ion mobility spectrometry/mass spectrometry (IMS/MS), X-ray diffraction, nuclear magnetic resonance (NMR), emission spectral analysis, polarography, ultraviolet-visual spectrometry, infrared spectrometry, and thin-layer chromatography.

In one aspect, the present specification provides for, and includes, a method of determining the NUE of a tobacco line using a metabolite signature comprising isolating a metabolite signature from a tobacco plant of a tobacco line, determining the amount of each metabolite comprising a metabolite signature, and determining the NUE of a tobacco line by comparing the metabolite signature to a control metabolite signature from a control tobacco line comprising a known NUE. In a further aspect of this method, NUE comprises enhanced NUE as compared to a control tobacco line. In another aspect of this method, a metabolite signature is isolated from a plant tissue selected from the group consisting of root tissue, leaf tissue, floral tissue, meristem tissue, and stem tissue.

In an aspect of a method provided herein, a metabolite signature comprises at least two metabolites. In a further aspect, a metabolite signature comprises at least three metabolites. In a further aspect, a metabolite signature comprises at least four metabolites. In a further aspect, a metabolite signature comprises at least five metabolites. In a further aspect, a metabolite signature comprises at least six metabolites. In a further aspect, a metabolite signature comprises at least seven metabolites. In a further aspect, a metabolite signature comprises at least eight metabolites. In a further aspect, a metabolite signature comprises at least nine metabolites. In a further aspect, a metabolite signature comprises at least ten metabolites. In a further aspect, a metabolite signature comprises at least eleven metabolites. In a further aspect, a metabolite signature comprises at least twelve metabolites. In a further aspect, a metabolite signature comprises at least thirteen metabolites. In a further aspect, a metabolite signature comprises at least fourteen metabolites. In a further aspect, a metabolite signature comprises at least fifteen metabolites. In a further aspect, a metabolite signature comprises at least twenty metabolites. In a further aspect, a metabolite signature comprises at least twenty-five metabolites. In a further aspect, a metabolite signature comprises at least thirty metabolites. In a further aspect, a metabolite signature comprises at least thirty-five metabolites. In a further aspect, a metabolite signature comprises at least forty metabolites. In a further aspect, a metabolite signature comprises at least forty-five metabolites. In a further aspect, a metabolite signature comprises at least fifty metabolites. In a further aspect, metabolite signature comprises between two and fifty metabolites. In a further aspect, metabolite signature comprises between three and forty-five metabolites. In a further aspect, metabolite signature comprises between three and forty metabolites. In a further aspect, metabolite signature comprises between four and thirty-five metabolites. In a further aspect, metabolite signature comprises between five and thirty metabolites. In a further aspect, metabolite signature comprises between six and twenty-five metabolites. In a further aspect, metabolite signature comprises between seven and twenty metabolites. In a further aspect, metabolite signature comprises between eight and fifteen metabolites. In a further aspect, metabolite signature comprises between nine and fourteen metabolites. In a further aspect, metabolite signature comprises between ten and thirteen metabolites. In a further aspect, metabolite signature comprises between ten and twelve metabolites.

In one aspect, the current specification provides for, and includes, a method of breeding a tobacco line comprising a metabolite signature associated with enhanced NUE comprising determining the metabolite signature of a first tobacco plant from a first tobacco line, where a first tobacco plant comprises enhanced NUE as compared to a control tobacco plant lacking the metabolite signature, crossing the first plant with a second plant of a second tobacco line, and obtaining at least one progeny seed from the crossing, where a progeny plant grown from at least one progeny seed comprises the metabolite signature, and where the progeny plant comprises enhanced NUE as compared to a control plant lacking the metabolite signature. In a further aspect of this method, a progeny plant is crossed to third plant that is from the first tobacco line. In another aspect, a first tobacco line is selected from the group consisting of MD609, MD601, Banket A1, K326, K346, K358, K394, K399, K730, NC196, NC37NF, NC471, NC55, NC92, NC2326, NC95, NC925. In another aspect, a second tobacco line is selected from the group consisting of TN86, TN86LC, TN90, TN90LC, TN97, TN97LC. In a further aspect, a metabolite signature comprises a leaf metabolite signature. In a further aspect, a metabolite signature comprises a root metabolite signature. In another aspect, a metabolite signature comprises higher amounts of 4-guanidinobutanoate, syringaldehyde, thiamin, p-hydroxybenzaldehyde, X-23454, X-23580, X-23852, or any combination thereof as compared to the metabolite signature of a control tobacco plant. In another aspect, a metabolite signature comprises lower amounts of X-2357, N-acetylmuramate, X-23319, X-23852, X-23330, alpha-ketoglutarate, X-21756, 4-hydroxy-2-oxoglutaric acid, X-23937, X-23916, 1-methyladenine, X-23453, X-11429, X-21796, N′-methylnicotinamide, cotinine, X-23389, N-acetylarginine, N-23366, N-acetylphenylalanine, naringenin, or any combination thereof as compared to the metabolite signature of a control tobacco plant.

In another aspect, a method provided herein comprises tobacco plants comprising enhanced NUE where enhanced NUE comprises an increased partial factor productivity (PFP) compared to a tobacco plant lacking enhanced NUE grown in the same conditions. In a further aspect, enhanced NUE comprises an increased agronomic efficiency (AE) compared to a tobacco plant lacking enhanced NUE grown in the same conditions. In a further aspect, enhanced NUE comprises an increased recovery efficiency (RE) compared to a tobacco plant lacking enhanced NUE grown in the same conditions. In a further aspect, enhanced NUE comprises an increased physiological efficiency (PE) compared to a tobacco plant lacking said enhanced NUE grown in the same conditions. In a further aspect, enhanced NUE comprises an increased internal efficiency (IE) compared to a tobacco plant lacking said enhanced NUE grown in the same conditions.

In one aspect, the present specification provides for, and includes, a method of selecting a tobacco plant comprising obtaining a population of tobacco plants, isolating at least one metabolite associated with enhanced NUE from at least one tobacco plant from the population of tobacco plants, and selecting at least one tobacco plant that comprises a higher amount of at least one metabolite as compared to a control tobacco plant. In a further aspect of this method, a selected tobacco plant comprises enhanced NUE as compared to a control tobacco plant. In a further aspect of this method, at least one metabolite is selected from the group consisting of 4-guanidinobutanoate, syringaldehyde, thiamin, p-hydroxybenzaldehyde, X-23454, X-23580, X-23852, or any combination thereof. In a further aspect of this method, a metabolite is isolated from a plant tissue selected from the group consisting of root tissue, leaf tissue, floral tissue, meristem tissue, and stem tissue.

In one aspect, the present specification provides for, and includes, a method of selecting a tobacco plant comprising obtaining a population of tobacco plants, isolating at least one metabolite associated with enhanced NUE from at least one tobacco plant from the population of tobacco plants, and selecting at least one tobacco plant that comprises a lower amount of at least one metabolite as compared to a control tobacco plant. In a further aspect of this method, a selected tobacco plant comprises a enhanced NUE as compared to a control tobacco plant. In a further aspect of this method, at least one metabolite is selected from the group consisting of X-2357, N-acetylmuramate, X-23319, X-23852, X-23330, alpha-ketoglutarate, X-21756, 4-hydroxy-2-oxoglutaric acid, X-23937, X-23916, 1-methyladenine, X-23453, X-11429, X-21796, N′-methylnicotinamide, cotinine, X-23389, N-acetylarginine, N-23366, N-acetylphenylalanine, naringenin, or any combination thereof. In a further aspect of this method, a metabolite is isolated from a plant tissue selected from the group consisting of root tissue, leaf tissue, floral tissue, meristem tissue, and stem tissue.

In one aspect, the present specification provides for, and includes, a method of screening a tobacco plant for a first metabolite signature associated with enhanced NUE comprising isolating a first metabolite signature from a tobacco plant, determining the amount of at least one metabolite that comprises that first metabolite signature, comparing the first metabolite signature to a second metabolite signature of a control tobacco plant comprising a known NUE, and determining if the first metabolite signature is associated with enhanced NUE.

In one aspect, the present specification provides for, and includes, a modified tobacco seed, or tobacco plant grown therefrom, comprising a cisgenic polynucleotide comprising a heterologous promoter operably linked to a coding region, where the modified tobacco plant comprises enhanced nitrogen utilization efficiency as compared to an unmodified control tobacco plant lacking the cisgenic polynucleotide when grown under the same conditions. In a further aspect, a modified tobacco seed or tobacco plant comprises a heterologous promoter that is selected from the group consisting of a constitutive promoter, an inducible promoter, a tissue-preferred promoter, and a tissue-specific promoter. In another aspect, a heterologous promoter comprises a polynucleotide sequence from a tobacco genome. In another aspect, a heterologous promoter comprises a polynucleotide sequence from a plant genome. In another aspect, a tissue-preferred promoter is a leaf-preferred promoter. In another aspect, a tissue-preferred promoter is a root-preferred promoter. In a further aspect, a modified tobacco seed or tobacco plant is of a Burley variety.

In a further aspect, a modified tobacco seed or tobacco plant of the present specification comprises lower amounts of TSNAs as compared to an unmodified tobacco plant lacking the cisgenic polynucleotide when grown under the same conditions. In a further aspect, a modified tobacco seed or tobacco plant comprises lower amount N′-nitrosonornicotine (NNN) as compared to an unmodified tobacco plant lacking the cisgenic polynucleotide when grown under the same conditions. In a further aspect, a modified tobacco seed or tobacco plant comprises lower amount 4-methylnitrosoamino-1-(3-pyridyl)-1-butanone (NNK) as compared to an unmodified tobacco plant lacking the cisgenic polynucleotide when grown under the same conditions. In a further aspect, a modified tobacco seed or tobacco plant comprises lower amount N′-nitrosoanatabine (NAT) as compared to an unmodified tobacco plant lacking the cisgenic polynucleotide when grown under the same conditions. In a further aspect, a modified tobacco seed or tobacco plant comprises lower amount N′-nitrosoanabasine (NAB) as compared to an unmodified tobacco plant lacking the cisgenic polynucleotide when grown under the same conditions. In a further aspect, a modified tobacco seed or tobacco plant comprises lower amounts of alkaloids as compared to an unmodified tobacco plant lacking the cisgenic polynucleotide when grown under the same conditions. In a further aspect, a modified tobacco seed or tobacco plant comprises lower amounts of nicotine as compared to an unmodified tobacco plant lacking the cisgenic polynucleotide when grown under the same conditions. In a further aspect, a modified tobacco seed or tobacco plant comprises lower amounts of nornicotine as compared to an unmodified tobacco plant lacking the cisgenic polynucleotide when grown under the same conditions. In a further aspect, a modified tobacco seed or tobacco plant comprises lower amounts of anabasine as compared to an unmodified tobacco plant lacking the cisgenic polynucleotide when grown under the same conditions. In a further aspect, a modified tobacco seed or tobacco plant comprises lower amounts of anatabine as compared to an unmodified tobacco plant lacking the cisgenic polynucleotide when grown under the same conditions.

In a further aspect, a modified tobacco seed or tobacco plant of the present specification comprises a coding region encoding a polypeptide that is at least 70% identical or similar to a sequence selected from the group consisting of SEQ ID NOs:1 to 8. In a further aspect, a modified tobacco seed or tobacco plant comprises a coding region encoding a polypeptide that is at least 75% identical or similar to a sequence selected from the group consisting of SEQ ID NOs:1 to 8. In a further aspect, a modified tobacco seed or tobacco plant comprises a coding region encoding a polypeptide that is at least 80% identical or similar to a sequence selected from the group consisting of SEQ ID NOs:1 to 8. In a further aspect, a modified tobacco seed or tobacco plant comprises a coding region encoding a polypeptide that is at least 85% identical or similar to a sequence selected from the group consisting of SEQ ID NOs:1 to 8. In a further aspect, a modified tobacco seed or tobacco plant comprises a coding region encoding a polypeptide that is at least 90% identical or similar to a sequence selected from the group consisting of SEQ ID NOs:1 to 8. In a further aspect, a modified tobacco seed or tobacco plant comprises a coding region encoding a polypeptide that is at least 95% identical or similar to a sequence selected from the group consisting of SEQ ID NOs:1 to 8. In a further aspect, a modified tobacco seed or tobacco plant comprises a coding region encoding a polypeptide that is at least 96% identical or similar to a sequence selected from the group consisting of SEQ ID NOs:1 to 8. In a further aspect, a modified tobacco seed or tobacco plant comprises a coding region encoding a polypeptide that is at least 97% identical or similar to a sequence selected from the group consisting of SEQ ID NOs:1 to 8. In a further aspect, a modified tobacco seed or tobacco plant comprises a coding region encoding a polypeptide that is at least 98% identical or similar to a sequence selected from the group consisting of SEQ ID NOs:1 to 8. In a further aspect, a modified tobacco seed or tobacco plant comprises a coding region encoding a polypeptide that is at least 99% identical or similar to a sequence selected from the group consisting of SEQ ID NOs:1 to 8. In a further aspect, a modified tobacco seed or tobacco plant comprises a coding region encoding a polypeptide that is 100% identical to a sequence selected from the group consisting of SEQ ID NOs:1 to 8.

In a further aspect, a modified tobacco seed or tobacco plant of the present specification comprises a coding region encoding a polynucleotide that is at least 70% identical or similar to a sequence selected from the group consisting of SEQ ID NOs:9 to 16. In a further aspect, a modified tobacco seed or tobacco plant comprises a coding region encoding a polynucleotide that is at least 75% identical or similar to a sequence selected from the group consisting of SEQ ID NOs:9 to 16. In a further aspect, a modified tobacco seed or tobacco plant comprises a coding region encoding a polynucleotide that is at least 80% identical or similar to a sequence selected from the group consisting of SEQ ID NOs:9 to 16. In a further aspect, a modified tobacco seed or tobacco plant comprises a coding region encoding a polynucleotide that is at least 85% identical or similar to a sequence selected from the group consisting of SEQ ID NOs:9 to 16. In a further aspect, a modified tobacco seed or tobacco plant comprises a coding region encoding a polynucleotide that is at least 90% identical or similar to a sequence selected from the group consisting of SEQ ID NOs:9 to 16. In a further aspect, a modified tobacco seed or tobacco plant comprises a coding region encoding a polynucleotide that is at least 95% identical or similar to a sequence selected from the group consisting of SEQ ID NOs:9 to 16. In a further aspect, a modified tobacco seed or tobacco plant comprises a coding region encoding a polynucleotide that is at least 96% identical or similar to a sequence selected from the group consisting of SEQ ID NOs:9 to 16. In a further aspect, a modified tobacco seed or tobacco plant comprises a coding region encoding a polynucleotide that is at least 97% identical or similar to a sequence selected from the group consisting of SEQ ID NOs:9 to 16. In a further aspect, a modified tobacco seed or tobacco plant comprises a coding region encoding a polynucleotide that is at least 98% identical or similar to a sequence selected from the group consisting of SEQ ID NOs:9 to 16. In a further aspect, a modified tobacco seed or tobacco plant comprises a coding region encoding a polynucleotide that is at least 99% identical or similar to a sequence selected from the group consisting of SEQ ID NOs:9 to 16. In a further aspect, a modified tobacco seed or tobacco plant comprises a coding region encoding a polynucleotide that is identical to a sequence selected from the group consisting of SEQ ID NOs:9 to 16.

In a further aspect, a modified tobacco seed or tobacco plant comprises a leaf-preferred promoter that is encoded by a sequence at least 70% identical to a sequence selected from the group consisting of SEQ ID NOs:17 to 19, or a functional fragment thereof. In a further aspect, a leaf-preferred promoter is encoded by a sequence at least 75% identical to a sequence selected from the group consisting of SEQ ID NOs:17 to 19, or a functional fragment thereof. In a further aspect, a leaf-preferred promoter is encoded by a sequence at least 80% identical to a sequence selected from the group consisting of SEQ ID NOs:17 to 19, or a functional fragment thereof. In a further aspect, a leaf-preferred promoter is encoded by a sequence at least 85% identical to a sequence selected from the group consisting of SEQ ID NOs:17 to 19, or a functional fragment thereof. In a further aspect, a leaf-preferred promoter is encoded by a sequence at least 90% identical to a sequence selected from the group consisting of SEQ ID NOs:17 to 19, or a functional fragment thereof. In a further aspect, a leaf-preferred promoter is encoded by a sequence at least 95% identical to a sequence selected from the group consisting of SEQ ID NOs:17 to 19, or a functional fragment thereof. In a further aspect, a leaf-preferred promoter is encoded by a sequence at least 96% identical to a sequence selected from the group consisting of SEQ ID NOs:17 to 19, or a functional fragment thereof. In a further aspect, a leaf-preferred promoter is encoded by a sequence at least 97% identical to a sequence selected from the group consisting of SEQ ID NOs:17 to 19, or a functional fragment thereof. In a further aspect, a leaf-preferred promoter is encoded by a sequence at least 98% identical to a sequence selected from the group consisting of SEQ ID NOs:17 to 19, or a functional fragment thereof. In a further aspect, a leaf-preferred promoter is encoded by a sequence at least 99% identical to a sequence selected from the group consisting of SEQ ID NOs:17 to 19, or a functional fragment thereof. In a further aspect, a leaf-preferred promoter is encoded by to a sequence selected from the group consisting of SEQ ID NOs:17 to 19, or a functional fragment thereof.

In a further aspect, a modified tobacco seed or tobacco plant comprises a root-preferred promoter that is encoded by a sequence at least 70% identical to a sequence selected from the group consisting of SEQ ID NOs:20 to 24, or a functional fragment thereof. In a further aspect, a leaf-preferred promoter is encoded by a sequence at least 75% identical to a sequence selected from the group consisting of SEQ ID NOs:20 to 24, or a functional fragment thereof. In a further aspect, a leaf-preferred promoter is encoded by a sequence at least 80% identical to a sequence selected from the group consisting of SEQ ID NOs:20 to 24, or a functional fragment thereof. In a further aspect, a leaf-preferred promoter is encoded by a sequence at least 85% identical to a sequence selected from the group consisting of SEQ ID NOs:20 to 24, or a functional fragment thereof. In a further aspect, a leaf-preferred promoter is encoded by a sequence at least 90% identical to a sequence selected from the group consisting of SEQ ID NOs:20 to 24, or a functional fragment thereof. In a further aspect, a leaf-preferred promoter is encoded by a sequence at least 95% identical to a sequence selected from the group consisting of SEQ ID NOs:20 to 24, or a functional fragment thereof. In a further aspect, a leaf-preferred promoter is encoded by a sequence at least 96% identical to a sequence selected from the group consisting of SEQ ID NOs:20 to 24, or a functional fragment thereof. In a further aspect, a leaf-preferred promoter is encoded by a sequence at least 97% identical to a sequence selected from the group consisting of SEQ ID NOs:20 to 24, or a functional fragment thereof. In a further aspect, a leaf-preferred promoter is encoded by a sequence at least 98% identical to a sequence selected from the group consisting of SEQ ID NOs:20 to 24, or a functional fragment thereof. In a further aspect, a leaf-preferred promoter is encoded by a sequence at least 99% identical to a sequence selected from the group consisting of SEQ ID NOs:20 to 24, or a functional fragment thereof. In a further aspect, a leaf-preferred promoter is encoded by to a sequence selected from the group consisting of SEQ ID NOs:20 to 24, or a functional fragment thereof.

In a further aspect, a modified tobacco plant of the present specification comprising a cisgenic polynucleotide comprises higher levels of a metabolite selected from the group consisting of 4-guanidinobutanoate, syringaldehyde, thiamin, and p-hydroxybenzaldehyde in root tissue as compared to an unmodified tobacco plant lacking the cisgenic polynucleotide when grown under the same conditions.

In a further aspect, a modified tobacco plant of the present specification comprising a cisgenic polynucleotide comprises higher levels of a metabolite selected from the group consisting of 4-guanidinobutanoate, X-23454, X-23580, and X-23852 in leaf tissue as compared to an unmodified tobacco plant lacking the cisgenic polynucleotide when grown under the same conditions.

In a further aspect, a modified tobacco plant of the present specification comprising a cisgenic polynucleotide comprises lower levels of a metabolite selected from the group consisting of X-2357, N-acetylmuramate, X-23319, X-23852, X-23330, alpha-ketoglutarate, X-21756, 4-hydroxy-2-oxoglutaric acid, X-23937, X-23916, and 1-methyladenine in root tissue as compared to an unmodified tobacco plant lacking the cisgenic polynucleotide when grown under the same conditions.

In a further aspect, a modified tobacco plant of the present specification comprising a cisgenic polynucleotide comprises lower levels of a metabolite selected from the group consisting of X-23453, X-21756, X-11429, X-21796, N′-methylnicotinamide, cotinine, X-23389, N-acetylarginine, N-23366, N-acetylphenylalanine, and naringenin in leaf tissue as compared to an unmodified tobacco plant lacking the cisgenic polynucleotide when grown under the same conditions.

In one aspect, the present specification provides for, and includes, a recombinant DNA construct comprising a heterologous promoter operably linked to a polynucleotide encoding a polypeptide at least 70% identical or similar to a polypeptide selected from the group consisting of SEQ ID NOs:1 to 8. In a further aspect, a recombinant DNA construct comprises a polynucleotide encoding a polypeptide at least 75% identical or similar to a polypeptide selected from the group consisting of SEQ ID NOs:1 to 8. In a further aspect, a recombinant DNA construct comprises a polynucleotide encoding a polypeptide at least 80% identical or similar to a polypeptide selected from the group consisting of SEQ ID NOs:1 to 8. In a further aspect, a recombinant DNA construct comprises a polynucleotide encoding a polypeptide at least 85% identical or similar to a polypeptide selected from the group consisting of SEQ ID NOs:1 to 8. In a further aspect, a recombinant DNA construct comprises a polynucleotide encoding a polypeptide at least 90% identical or similar to a polypeptide selected from the group consisting of SEQ ID NOs:1 to 8. In a further aspect, a recombinant DNA construct comprises a polynucleotide encoding a polypeptide at least 95% identical or similar to a polypeptide selected from the group consisting of SEQ ID NOs:1 to 8. In a further aspect, a recombinant DNA construct comprises a polynucleotide encoding a polypeptide at least 96% identical or similar to a polypeptide selected from the group consisting of SEQ ID NOs:1 to 8. In a further aspect, a recombinant DNA construct comprises a polynucleotide encoding a polypeptide at least 97% identical or similar to a polypeptide selected from the group consisting of SEQ ID NOs:1 to 8. In a further aspect, a recombinant DNA construct comprises a polynucleotide encoding a polypeptide at least 98% identical or similar to a polypeptide selected from the group consisting of SEQ ID NOs:1 to 8. In a further aspect, a recombinant DNA construct comprises a polynucleotide encoding a polypeptide at least 99% identical or similar to a polypeptide selected from the group consisting of SEQ ID NOs:1 to 8. In a further aspect, a recombinant DNA construct comprises a polynucleotide encoding a polypeptide 100% identical to a polypeptide selected from the group consisting of SEQ ID NOs:1 to 8.

In one aspect, the present specification provides for, and includes, cured tobacco material, or a tobacco product comprising the cured tobacco material, where the cured tobacco material is made from a tobacco plant comprising a cisgenic polynucleotide comprising a heterologous promoter operably linked to a coding region, where the modified tobacco plant comprises enhanced nitrogen utilization efficiency as compared to an unmodified control tobacco plant lacking the cisgenic polynucleotide when grown under the same conditions.

In one aspect, the present specification provides for, and includes, a greenhouse, growth chamber, or field comprising the modified tobacco seed or plant disclosed herein. In one aspect, the present specification provides for, and includes, a method to grow tobacco plants of the present specification in a greenhouse, growth chamber, or field.

In one aspect, the present specification provides for, and includes, a modified tobacco seed, or tobacco plant grown therefrom, comprising at least one mutation in an endogenous locus encoding a polypeptide selected from the group consisting of SEQ ID NOs: 25 to 40, and where a modified tobacco seed or tobacco plant comprises enhanced nitrogen utilization efficiency as compared to an unmodified control tobacco plant lacking at least one mutation when grown under the same conditions. In a further aspect, a mutation in an endogenous locus is selected from the group consisting of an insertion, a deletion, a substitution, and an inversion. In another aspect, a mutation in an endogenous locus is a silent mutation, a non-silent mutation, or a null mutation. In a further aspect, a modified tobacco seed or modified tobacco plant is of a Burley variety.

In a further aspect, a modified tobacco plant comprises higher levels of a metabolite selected from the group consisting of 4-guanidinobutanoate, syringaldehyde, thiamin, and p-hydroxybenzaldehyde in root tissue as compared to an unmodified tobacco plant when grown under the same conditions. In a further aspect, a modified tobacco plant comprises higher levels of a metabolite selected from the group consisting of 4-guanidinobutanoate, X-23454, X-23580, and X-23852 in leaf tissue as compared to an unmodified tobacco plant when grown under the same conditions. In a further aspect, a modified tobacco plant comprises lower levels of a metabolite selected from the group consisting of X-2357, N-acetylmuramate, X-23319, X-23852, X-23330, alpha-ketoglutarate, X-21756, 4-hydroxy-2-oxoglutaric acid, X-23937, X-23916, and 1-methyladenine in root tissue as compared to an unmodified tobacco plant lacking when grown under the same conditions. In a further aspect, a modified tobacco plant comprises lower levels of a metabolite selected from the group consisting of X-23453, X-21756, X-11429, X-21796, N′-methylnicotinamide, cotinine, X-23389, N-acetylarginine, N-23366, N-acetylphenylalanine, and naringenin in leaf tissue as compared to an unmodified tobacco plant when grown under the same conditions.

In one aspect, the present specification provides for, and includes, a recombinant DNA construct comprising a heterologous promoter operably linked to a guide RNA comprising at least 18 contiguous nucleotides identical or complementary to a polynucleotide encoding a polypeptide selected from the group consisting of SEQ ID NOs:25 to 40. In a further aspect, a guide RNA comprises at least 19 contiguous nucleotides identical or complementary to a polynucleotide encoding a polypeptide selected from the group consisting of SEQ ID NOs:25 to 40. In a further aspect, a guide RNA comprises at least 20 contiguous nucleotides identical or complementary to a polynucleotide encoding a polypeptide selected from the group consisting of SEQ ID NOs:25 to 40. In a further aspect, a guide RNA comprises at least 21 contiguous nucleotides identical or complementary to a polynucleotide encoding a polypeptide selected from the group consisting of SEQ ID NOs:25 to 40. In a further aspect, a guide RNA comprises at least 22 contiguous nucleotides identical or complementary to a polynucleotide encoding a polypeptide selected from the group consisting of SEQ ID NOs:25 to 40. In a further aspect, a guide RNA comprises at least 23 contiguous nucleotides identical or complementary to a polynucleotide encoding a polypeptide selected from the group consisting of SEQ ID NOs:25 to 40. In a further aspect, a guide RNA comprises at least 24 contiguous nucleotides identical or complementary to a polynucleotide encoding a polypeptide selected from the group consisting of SEQ ID NOs:25 to 40. In a further aspect, a guide RNA comprises at least 25 contiguous nucleotides identical or complementary to a polynucleotide encoding a polypeptide selected from the group consisting of SEQ ID NOs:25 to 40. In a further aspect, a guide RNA comprises at least 26 contiguous nucleotides identical or complementary to a polynucleotide encoding a polypeptide selected from the group consisting of SEQ ID NOs:25 to 40. In a further aspect, a guide RNA comprises at least 27 contiguous nucleotides identical or complementary to a polynucleotide encoding a polypeptide selected from the group consisting of SEQ ID NOs:25 to 40. In a further aspect, a guide RNA comprises at least 28 contiguous nucleotides 100% identical or complementary to a polynucleotide encoding a polypeptide selected from the group consisting of SEQ ID NOs:25 to 40.

In one aspect, the present specification provides for, and includes, cured tobacco material, or a tobacco product comprising the cured tobacco material, where the cured tobacco material is made from a tobacco plant comprising at least one mutation in an endogenous locus encoding a polypeptide selected from the group consisting of SEQ ID NOs:25 to 40, and where the modified tobacco seed or tobacco plant comprises enhanced NUE as compared to an unmodified control tobacco plant lacking at least one mutation when grown under the same conditions. In a further aspect, a tobacco plant comprises at least two mutations in an endogenous locus encoding a polypeptide selected from the group consisting of SEQ ID NOs:25 to 40. In a further aspect, a tobacco plant comprises at least three mutations in an endogenous locus encoding a polypeptide selected from the group consisting of SEQ ID NOs:25 to 40. In a further aspect, a tobacco plant comprises at least four mutations in an endogenous locus encoding a polypeptide selected from the group consisting of SEQ ID NOs:25 to 40. In a further aspect, a tobacco plant comprises at least five mutations in an endogenous locus encoding a polypeptide selected from the group consisting of SEQ ID NOs:25 to 40. In a further aspect, a tobacco plant comprises at least six mutations in an endogenous locus encoding a polypeptide selected from the group consisting of SEQ ID NOs:25 to 40. In a further aspect, a tobacco plant comprises at least seven mutations in an endogenous locus encoding a polypeptide selected from the group consisting of SEQ ID NOs:25 to 40. In a further aspect, a tobacco plant comprises at least eight mutations in an endogenous locus encoding a polypeptide selected from the group consisting of SEQ ID NOs:25 to 40. In a further aspect, a tobacco plant comprises at least nine mutations in an endogenous locus encoding a polypeptide selected from the group consisting of SEQ ID NOs:25 to 40. In a further aspect, a tobacco plant comprises at least ten mutations in an endogenous locus encoding a polypeptide selected from the group consisting of SEQ ID NOs:25 to 40.

In one aspect, the present specification provides for, and includes, a modified tobacco seed, or tobacco plant grown therefrom, comprising a cisgenic polynucleotide comprising a heterologous promoter operably linked to a polynucleotide encoding a small RNA (sRNA) at least 85% identical or complementary to a polynucleotide selected from the group consisting of SEQ ID NOs:41 to 56, and where the modified tobacco seed or tobacco plant comprises enhanced NUE as compared to an unmodified control tobacco plant lacking the cisgenic polynucleotide when grown under the same conditions. In a further aspect, a cisgenic polynucleotide comprises a polynucleotide encoding a sRNA at least 90% identical or complementary to a polynucleotide selected from the group consisting of SEQ ID NOs:41 to 56. In a further aspect, a cisgenic polynucleotide comprises a polynucleotide encoding a sRNA at least 91% identical or complementary to a polynucleotide selected from the group consisting of SEQ ID NOs:41 to 56. In a further aspect, a cisgenic polynucleotide comprises a polynucleotide encoding a sRNA at least 92% identical or complementary to a polynucleotide selected from the group consisting of SEQ ID NOs:41 to 56. In a further aspect, a cisgenic polynucleotide comprises a polynucleotide encoding a sRNA at least 93% identical or complementary to a polynucleotide selected from the group consisting of SEQ ID NOs:41 to 56. In a further aspect, a cisgenic polynucleotide comprises a polynucleotide encoding a sRNA at least 94% identical or complementary to a polynucleotide selected from the group consisting of SEQ ID NOs:41 to 56. In a further aspect, a cisgenic polynucleotide comprises a polynucleotide encoding a sRNA at least 95% identical or complementary to a polynucleotide selected from the group consisting of SEQ ID NOs:41 to 56. In a further aspect, a cisgenic polynucleotide comprises a polynucleotide encoding a sRNA at least 96% identical or complementary to a polynucleotide selected from the group consisting of SEQ ID NOs:41 to 56. In a further aspect, a cisgenic polynucleotide comprises a polynucleotide encoding a sRNA at least 97% identical or complementary to a polynucleotide selected from the group consisting of SEQ ID NOs:41 to 56. In a further aspect, a cisgenic polynucleotide comprises a polynucleotide encoding a sRNA at least 98% identical or complementary to a polynucleotide selected from the group consisting of SEQ ID NOs:41 to 56. In a further aspect, a cisgenic polynucleotide comprises a polynucleotide encoding a sRNA at least 99% identical or complementary to a polynucleotide selected from the group consisting of SEQ ID NOs:41 to 56. In a further aspect, a cisgenic polynucleotide comprises a polynucleotide encoding a sRNA 100% identical or complementary to a polynucleotide selected from the group consisting of SEQ ID NOs:41 to 56. In a further aspect, a heterologous promoter is selected from the group consisting of a constitutive promoter, an inducible promoter, a tissue-preferred promoter, and a tissue-specific promoter. In a further aspect, a tissue-preferred promoter is a leaf-preferred promoter. In a further aspect, a tissue-preferred promoter is a root-preferred promoter.

In a further aspect, a sRNA having at least 18 nucleotides. In a further aspect, a sRNA comprises at least 19 nucleotides. In a further aspect, a sRNA comprises at least 20 nucleotides. In a further aspect, a sRNA comprises at least 21 nucleotides. In a further aspect, a sRNA comprises at least 22 nucleotides. In a further aspect, a sRNA comprises at least 23 nucleotides. In a further aspect, a sRNA comprises at least 24 nucleotides. In a further aspect, a sRNA comprises at least 25 nucleotides. In a further aspect, a sRNA comprises at least 26 nucleotides. In a further aspect, a sRNA comprises at least 27 nucleotides. In a further aspect, a sRNA comprises at least 28 nucleotides. In a further aspect, a sRNA is selected from the group consisting of a microRNA, a small-interfering RNA (siRNA), a trans-acting siRNA, and precursors thereof. In a further aspect, a sRNA down-regulates the expression or translation of a polynucleotide selected from the group consisting of SEQ ID NOs:41 to 56.

In one aspect, the present specification provides for, and includes, a recombinant DNA construct comprising a heterologous promoter operably linked to a polynucleotide encoding a small RNA (sRNA) at least 85% identical or complementary to a polynucleotide selected from the group consisting of SEQ ID NOs:41 to 56. In a further aspect, a recombinant DNA construct comprises a polynucleotide encoding a sRNA at least 90% identical or complementary to a polynucleotide selected from the group consisting of SEQ ID NOs:41 to 56. In a further aspect, a recombinant DNA construct comprises a polynucleotide encoding a sRNA at least 91% identical or complementary to a polynucleotide selected from the group consisting of SEQ ID NOs:41 to 56. In a further aspect, a recombinant DNA construct comprises a polynucleotide encoding a sRNA at least 92% identical or complementary to a polynucleotide selected from the group consisting of SEQ ID NOs:41 to 56. In a further aspect, a recombinant DNA construct comprises a polynucleotide encoding a sRNA at least 93% identical or complementary to a polynucleotide selected from the group consisting of SEQ ID NOs:41 to 56. In a further aspect, a recombinant DNA construct comprises a polynucleotide encoding a sRNA at least 94% identical or complementary to a polynucleotide selected from the group consisting of SEQ ID NOs:41 to 56. In a further aspect, a recombinant DNA construct comprises a polynucleotide encoding a sRNA at least 95% identical or complementary to a polynucleotide selected from the group consisting of SEQ ID NOs:41 to 56. In a further aspect, a recombinant DNA construct comprises a polynucleotide encoding a sRNA at least 96% identical or complementary to a polynucleotide selected from the group consisting of SEQ ID NOs:41 to 56. In a further aspect, a recombinant DNA construct comprises a polynucleotide encoding a sRNA at least 97% identical or complementary to a polynucleotide selected from the group consisting of SEQ ID NOs:41 to 56. In a further aspect, a recombinant DNA construct comprises a polynucleotide encoding a sRNA at least 98% identical or complementary to a polynucleotide selected from the group consisting of SEQ ID NOs:41 to 56. In a further aspect, a recombinant DNA construct comprises a polynucleotide encoding a sRNA at least 99% identical or complementary to a polynucleotide selected from the group consisting of SEQ ID NOs:41 to 56. In a further aspect, a recombinant DNA construct comprises a polynucleotide encoding a sRNA 100% identical or complementary to a polynucleotide selected from the group consisting of SEQ ID NOs:41 to 56.

In one aspect, the present specification provides for, and includes, cured tobacco material, or a tobacco product comprising the cured tobacco material, where the cured tobacco material is made from a tobacco plant comprising a cisgenic polynucleotide comprising a heterologous promoter operably linked to a polynucleotide encoding a sRNA at least 85% identical or complementary to a polynucleotide selected from the group consisting of SEQ ID NOs:41 to 56, and where the modified tobacco seed or tobacco plant comprises enhanced NUE as compared to an unmodified control tobacco plant lacking the cisgenic polynucleotide when grown under the same conditions. In a further aspect, a cisgenic polynucleotide encodes a sRNA at least 90% identical or complementary to a polynucleotide selected from the group consisting of SEQ ID NOs:41 to 56. In a further aspect, a cisgenic polynucleotide encodes a sRNA at least 91% identical or complementary to a polynucleotide selected from the group consisting of SEQ ID NOs:41 to 56. In a further aspect, a cisgenic polynucleotide encodes a sRNA at least 92% identical or complementary to a polynucleotide selected from the group consisting of SEQ ID NOs:41 to 56. In a further aspect, a cisgenic polynucleotide encodes a sRNA at least 93% identical or complementary to a polynucleotide selected from the group consisting of SEQ ID NOs:41 to 56. In a further aspect, a cisgenic polynucleotide encodes a sRNA at least 94% identical or complementary to a polynucleotide selected from the group consisting of SEQ ID NOs:41 to 56. In a further aspect, a cisgenic polynucleotide encodes a sRNA at least 95% identical or complementary to a polynucleotide selected from the group consisting of SEQ ID NOs:41 to 56. In a further aspect, a cisgenic polynucleotide encodes a sRNA at least 96% identical or complementary to a polynucleotide selected from the group consisting of SEQ ID NOs:41 to 56. In a further aspect, a cisgenic polynucleotide encodes a sRNA at least 97% identical or complementary to a polynucleotide selected from the group consisting of SEQ ID NOs:41 to 56. In a further aspect, a cisgenic polynucleotide encodes a sRNA at least 98% identical or complementary to a polynucleotide selected from the group consisting of SEQ ID NOs:41 to 56. In a further aspect, a cisgenic polynucleotide encodes a sRNA at least 99% identical or complementary to a polynucleotide selected from the group consisting of SEQ ID NOs:41 to 56. In a further aspect, a cisgenic polynucleotide encodes a sRNA 100% identical or complementary to a polynucleotide selected from the group consisting of SEQ ID NOs:41 to 56.

In one aspect, the present specification provides for, and includes, a method of enhancing the NUE of a tobacco plant comprising introducing a cisgenic nucleic acid molecule into a tobacco cell, and regenerating a modified tobacco plant from that tobacco cell where the modified tobacco plant comprises enhanced NUE as compared to a tobacco plant lacking the cisgenic nucleic acid molecule. In another aspect, the method further comprises crossing the modified tobacco plant with a second tobacco plant or self-pollinating the modified tobacco plant.

In one aspect, the present specification provides for, and includes, a method of enhancing the NUE of a tobacco plant comprising introducing a modification to a nucleic acid molecule encoding a gene having a sequence selected from the group consisting of SEQ ID NOs:41 to 56 in a tobacco cell and regenerating a modified tobacco plant from the tobacco cell, where the modified tobacco plant comprises enhanced NUE as compared to a tobacco plant lacking the modification. In another aspect, the method further comprises crossing the modified tobacco plant with a second tobacco plant or self-pollinating the modified tobacco plant. In a further aspect, a modification is introducing via a method comprising the use of an RNA-guided nuclease. In a further aspect, a RNA-guided nuclease is selected from the group consisting of a Cas9 nuclease, a Cpf1 nuclease, a CasX nuclease, a CasY nuclease, and functional homologues thereof. In a further aspect, the modification is selected from the group consisting of an insertion, a substitution, an inversion, and a deletion

In one aspect, the present specification provides for, and includes, a method of enhancing the NUE of a tobacco plant comprising introducing a nucleic acid encoding a small RNA (sRNA) homologous to at least 18 contiguous nucleic acids of a nucleic acid molecule encoding a gene having a sequence selected from the group consisting of SEQ ID NOs.41 to 56 in a tobacco cell, and regenerating a modified tobacco plant from the tobacco cell, where the modified tobacco plant comprises enhanced NUE as compared to a tobacco plant lacking the sRNA. In another aspect, the method further comprises crossing the modified tobacco plant with a second tobacco plant or self-pollinating the modified tobacco plant. In a further aspect, the method comprises introducing a sRNA selected from the group consisting of a microRNA, a small-interfering RNA (siRNA), a trans-acting siRNA, and precursors thereof.

In one aspect, the present specification provides for, and includes, a method comprising providing a first population of tobacco plants comprising enhanced NUE, genotyping a first population of tobacco plants for the presence of a molecular marker within 20 cM of an enhanced NUE locus; and selecting one or more tobacco plants genotyped and found to comprise the molecular marker. In a further aspect, a method disclosed herein comprises genotyping a first population of tobacco plants for the presence of a molecular marker within 15 cM of an enhanced NUE locus. In a further aspect, a method disclosed herein comprises genotyping a first population of tobacco plants for the presence of a molecular marker within 10 cM of an enhanced NUE locus. In a further aspect, a method disclosed herein comprises genotyping a first population of tobacco plants for the presence of a molecular marker within 9 cM of an enhanced NUE locus. In a further aspect, a method disclosed herein comprises genotyping a first population of tobacco plants for the presence of a molecular marker within 8 cM of an enhanced NUE locus. In a further aspect, a method disclosed herein comprises genotyping a first population of tobacco plants for the presence of a molecular marker within 7 cM of an enhanced NUE locus. In a further aspect, a method disclosed herein comprises genotyping a first population of tobacco plants for the presence of a molecular marker within 6 cM of an enhanced NUE locus. In a further aspect, a method disclosed herein comprises genotyping a first population of tobacco plants for the presence of a molecular marker within 5 cM of an enhanced NUE locus. In a further aspect, a method disclosed herein comprises genotyping a first population of tobacco plants for the presence of a molecular marker within 4 cM of an enhanced NUE locus. In a further aspect, a method disclosed herein comprises genotyping a first population of tobacco plants for the presence of a molecular marker within 3 cM of an enhanced NUE locus. In a further aspect, a method disclosed herein comprises genotyping a first population of tobacco plants for the presence of a molecular marker within 2 cM of an enhanced NUE locus. In a further aspect, a method disclosed herein comprises genotyping a first population of tobacco plants for the presence of a molecular marker within 1 cM of an enhanced NUE locus. In a further aspect, a method disclosed herein comprises genotyping a first population of tobacco plants for the presence of a molecular marker within 0.5 cM of an enhanced NUE locus. In a further aspect, the method comprises crossing one or more selected tobacco plants to a second tobacco plant; and obtaining progeny seed from that cross. In a further aspect, a molecular marker is selected from the group consisting of a SNP marker, an INDEL marker, an RFLP marker, an SSR marker, an AFLP marker, and a RAPD marker.

In a further aspect, a method provided herein comprises a tobacco plant comprising an enhanced NUE locus comprising a polynucleotide encoding a polypeptide at least 70% identical or similar to a polypeptide selected from the group consisting of SEQ ID NOs:1 to 8. In a further aspect, a polynucleotide encodes a polypeptide at least 75% identical or similar to a polypeptide selected from the group consisting of SEQ ID NOs:1 to 8. In a further aspect, a polynucleotide encodes a polypeptide at least 80% identical or similar to a polypeptide selected from the group consisting of SEQ ID NOs:1 to 8. In a further aspect, a polynucleotide encodes a polypeptide at least 85% identical or similar to a polypeptide selected from the group consisting of SEQ ID NOs:1 to 8. In a further aspect, a polynucleotide encodes a polypeptide at least 90% identical or similar to a polypeptide selected from the group consisting of SEQ ID NOs:1 to 8. In a further aspect, a polynucleotide encodes a polypeptide at least 95% identical or similar to a polypeptide selected from the group consisting of SEQ ID NOs:1 to 8. In a further aspect, a polynucleotide encodes a polypeptide at least 96% identical or similar to a polypeptide selected from the group consisting of SEQ ID NOs:1 to 8. In a further aspect, a polynucleotide encodes a polypeptide at least 97% identical or similar to a polypeptide selected from the group consisting of SEQ ID NOs:1 to 8. In a further aspect, a polynucleotide encodes a polypeptide at least 98% identical or similar to a polypeptide selected from the group consisting of SEQ ID NOs:1 to 8. In a further aspect, a polynucleotide encodes a polypeptide at least 99% identical or similar to a polypeptide selected from the group consisting of SEQ ID NOs:1 to 8. In a further aspect, a polynucleotide encodes a polypeptide 100% identical to a polypeptide selected from the group consisting of SEQ ID NOs:1 to 8. In a further aspect, an enhanced NUE locus is genetically linked to a polynucleotide sequence selected from the group consisting of SEQ ID NOs:57 to 64. In another aspect, an enhanced NUE locus is genetically linked to a G nucleotide at position 57 of SEQ ID NOs: 58. In another aspect, an enhanced NUE locus is genetically linked to a C nucleotide at position 117 of SEQ ID NOs: 58. In another aspect, an enhanced NUE locus is genetically linked to a G nucleotide at position 57 and a C nucleotide at position 117 of SEQ ID NOs: 58. In another aspect, an enhanced NUE locus is genetically linked to a T nucleotide at position 147 of SEQ ID NO:57. In another aspect, an enhanced NUE locus is genetically linked to a G nucleotide at position 162 of SEQ ID NO:59. In another aspect, an enhanced NUE locus is genetically linked to a C nucleotide at position 36 of SEQ ID NO:60. In another aspect, an enhanced NUE locus is genetically linked to a T nucleotide at position 36 of SEQ ID NO:61. In another aspect, an enhanced NUE locus is genetically linked to a T nucleotide at position 36 of SEQ ID NO:62. In another aspect, an enhanced NUE locus is genetically linked to a G nucleotide at position 36 of SEQ ID NO:63. In another aspect, an enhanced NUE locus is genetically linked to a T nucleotide at position 36 of SEQ ID NO:64.

In a further aspect of a method provided herein, a first population of tobacco plants is of a Maryland variety. In a further aspect, a method provided herein comprises a first population of tobacco plants of a variety selected from the group consisting of MD609, MD601, Banket A1, K326, K346, K358, K394, K399, K730, NC196, NC37NF, NC471, NC55, NC92, NC2326, NC95, NC925. In a further aspect, a method provided herein comprises a second population of tobacco plants of the Burley variety In a further aspect, a method provided herein comprises a second population of tobacco plants of a variety selected from the group consisting of TN86, TN86LC, TN90, TN90LC, TN97, TN97LC.

In a further aspect, a method provided herein comprises progeny seed comprising molecular markers. In a further aspect, a method provided herein comprises progeny seed comprising enhanced NUE. In a further aspect, a method provided herein comprises progeny seed comprising a molecular marker within 20 cM of an enhanced NUE efficiency locus provided herein. In a further aspect, a method provided herein comprises progeny seed comprising a molecular marker within 15 cM of an enhanced NUE efficiency locus provided herein. In a further aspect, a method provided herein comprises progeny seed comprising a molecular marker within 10 cM of an enhanced NUE efficiency locus provided herein. In a further aspect, a method provided herein comprises progeny seed comprising a molecular marker within 9 cM of an enhanced NUE efficiency locus provided herein. In a further aspect, a method provided herein comprises progeny seed comprising a molecular marker within 8 cM of an enhanced NUE efficiency locus provided herein. In a further aspect, a method provided herein comprises progeny seed comprising a molecular marker within 7 cM of an enhanced NUE efficiency locus provided herein. In a further aspect, a method provided herein comprises progeny seed comprising a molecular marker within 6 cM of an enhanced NUE efficiency locus provided herein. In a further aspect, a method provided herein comprises progeny seed comprising a molecular marker within 5 cM of an enhanced NUE efficiency locus provided herein. In a further aspect, a method provided herein comprises progeny seed comprising a molecular marker within 4 cM of an enhanced NUE efficiency locus provided herein. In a further aspect, a method provided herein comprises progeny seed comprising a molecular marker within 3 cM of an enhanced NUE efficiency locus provided herein. In a further aspect, a method provided herein comprises progeny seed comprising a molecular marker within 2 cM of an enhanced NUE efficiency locus provided herein. In a further aspect, a method provided herein comprises progeny seed comprising a molecular marker within 1 cM of an enhanced NUE efficiency locus provided herein. In a further aspect, a method provided herein comprises progeny seed comprising a molecular marker within 0.5 cM of an enhanced NUE efficiency locus provided herein.

In one aspect, the present specification provides for, and includes, a method comprising providing a first population of tobacco plants, genotyping the first population of tobacco plants for the presence of an enhanced NUE allele of a locus encoded by a sequence selected from the group consisting of SEQ ID NOs:9 to 16; and selecting one or more genotyped tobacco plants that comprise an enhanced NUE allele. In a further aspect, the method further comprises crossing the one or more selected tobacco plants to a second tobacco plant; and obtaining progeny seed from the cross.

In one aspect, the present specification provides for, and includes, a method of introgressing an enhanced NUE trait into a tobacco variety comprising crossing a first tobacco variety comprising an enhanced nitrogen utilization efficiency trait with a second tobacco variety lacking the enhanced nitrogen utilization efficiency trait, obtaining progeny seed from the cross, genotyping at least one progeny seed for a molecular marker linked to an enhanced nitrogen utilization efficiency trait, where the molecular marker is within 20 cM of a locus having a sequence selected from the group consisting of SEQ ID NOs:9 to 16; and selecting a progeny seed comprising an enhanced nitrogen utilization efficiency trait.

In one aspect, the present specification provides for, and includes, a method of selecting a tobacco plant with an enhanced NUE trait comprising isolating nucleic acids from a collection of tobacco germplasm, assaying the isolated nucleic acids for one or more markers located within 20 cM of a locus having a sequence selected from the group consisting of SEQ ID NOs:9 to 16, and selecting a tobacco plant comprising an enhanced NUE trait. In a further aspect, the method further comprises crossing the one or more selected tobacco plants to a second tobacco plant; and obtaining progeny seed from the cross.

In one aspect, the present specification provides for, and includes, a method of selecting a tobacco plant with an enhanced NUE trait comprising isolating nucleic acids from a collection of tobacco germplasm, assaying the isolated nucleic acids for one or more markers located within 20 cM of a marker selected from the group consisting of SEQ ID NOs:57 to 64, and selecting a tobacco plant comprising an enhanced NUE trait. In a further aspect, a method disclosed herein comprises assaying isolated nucleic acids for one or more markers located within 15 cM of a marker selected from the group consisting of SEQ ID NOs: 58. In a further aspect, a method disclosed herein comprises assaying isolated nucleic acids for one or more markers located within 10 cM of a marker selected from the group consisting of SEQ ID NOs:57 to 64. In a further aspect, a method disclosed herein comprises assaying isolated nucleic acids for one or more markers located within 9 cM of a marker selected from the group consisting of SEQ ID NOs:57 to 64. In a further aspect, a method disclosed herein comprises assaying isolated nucleic acids for one or more markers located within 8 cM of a marker selected from the group consisting of SEQ ID NOs:57 to 64. In a further aspect, a method disclosed herein comprises assaying isolated nucleic acids for one or more markers located within 7 cM of a marker selected from the group consisting of SEQ ID NOs:57 to 64. In a further aspect, a method disclosed herein comprises assaying isolated nucleic acids for one or more markers located within 6 cM of a marker selected from the group consisting of SEQ ID NOs:57 to 64. In a further aspect, a method disclosed herein comprises assaying isolated nucleic acids for one or more markers located within 5 cM of a marker selected from the group consisting of SEQ ID NOs:57 to 64. In a further aspect, a method disclosed herein comprises assaying isolated nucleic acids for one or more markers located within 4 cM of a marker selected from the group consisting of SEQ ID NOs:57 to 64. In a further aspect, a method disclosed herein comprises assaying isolated nucleic acids for one or more markers located within 3 cM of a marker selected from the group consisting of SEQ ID NOs:57 to 64. In a further aspect, a method disclosed herein comprises assaying isolated nucleic acids for one or more markers located within 2 cM of a marker selected from the group consisting of SEQ ID NOs:57 to 64. In a further aspect, a method disclosed herein comprises assaying isolated nucleic acids for one or more markers located within 1 cM of a marker selected from the group consisting of SEQ ID NOs:57 to 64. In a further aspect, a method disclosed herein comprises assaying isolated nucleic acids for one or more markers located within 0.5 cM of a marker selected from the group consisting of SEQ ID NOs:57 to 64. In a further aspect, a method disclosed herein comprises assaying isolated nucleic acids for a marker selected from the group consisting of SEQ ID NOs:57 to 64. In another aspect, an allele associated with enhanced NUE comprises a G nucleotide at position 57 of SEQ ID NO:58. In another aspect an allele associated with enhanced NUE comprises a C nucleotide at position 117 of SEQ ID NO:58. In another aspect, an allele associated with enhanced NUE comprises a G nucleotide at position 57 and a C nucleotide at position 117 of SEQ ID NO:58. In another aspect, an allele associated with enhanced NUE comprises a T nucleotide at position 147 of SEQ ID NO:57. In another aspect, an allele associated with enhanced NUE comprises a G nucleotide at position 162 of SEQ ID NO:59. In another aspect, an allele associated with enhanced NUE comprises a C nucleotide at position 36 of SEQ ID NO:60. In another aspect, an allele associated with enhanced NUE comprises a T nucleotide at position 36 of SEQ ID NO:61. In another aspect, an allele associated with enhanced NUE comprises a T nucleotide at position 36 of SEQ ID NO:62. In another aspect, an allele associated with enhanced NUE comprises a G nucleotide at position 36 of SEQ ID NO:63. In another aspect, an allele associated with enhanced NUE comprises a T nucleotide at position 36 of SEQ ID NO:64. In a further aspect, a tobacco plant can be selected comprising any combination of alleles associated with enhanced NUE disclosed herein.

The following are exemplary embodiments

Embodiment 1. A method of determining the nitrogen utilization efficiency (NUE) of a tobacco line comprising:
a. obtaining at least one metabolite from a tobacco plant of said tobacco line;
b. determining the amount of said at least one metabolite; and
c. determining the nitrogen utilization efficiency of said tobacco line based on the amount of said at least one metabolite identified in step (b).

Embodiment 2. The method of embodiment 1, wherein said at least one metabolite is obtained from a plant tissue selected from the group consisting of root tissue, leaf tissue, floral tissue, meristem tissue, and stem tissue.

Embodiment 3. The method of embodiment 1 or 2, wherein said plant tissue comprises leaf tissue.

Embodiment 4. The method of any one of embodiments 1 to 3, wherein said plant tissue comprises root tissue.

Embodiment 5. The method of any one of embodiments 1 to 4, wherein said at least one metabolite is selected from the group consisting of X-2357, N-acetylmuramate, X-23319, X-23852, X-23330, alpha-ketoglutarate, X-21756, 4-hydroxy-2-oxoglutaric acid, D-23937, X-23937, X-23916, 1-methyladenine, 4-guanidinobutanoate, syringaldehyde, thiamin, p-hydroxybenzaldehyde, X-23453, X-11429, X-21796, N′-methylnicotinamide, cotinine, X-23389, N-acetylarginine, X-23366, N-acetylphenylalanine, naringenin, X-23454, X-23580, and X-23852.

Embodiment 6. The method of any one of embodiments 1 to 5, wherein said NUE comprises enhanced NUE as compared to a tobacco line that comprises a lower amount of said at least one metabolite in said at least one tissue.

Embodiment 7. The method of any one of embodiments 1 to 6, wherein said NUE comprises enhanced NUE as compared to a tobacco line that comprises an equal amount of said at least one metabolite in said at least one tissue.

Embodiment 8. The method of any one of embodiments 1 to 7, wherein said NUE comprises enhanced NUE as compared to a tobacco line that comprises an equal amount of said at least one metabolite in said at least one tissue.

Embodiment 9. The method of any one of embodiments 1 to 8, wherein said NUE comprises decreased NUE as compared to a tobacco line that comprises a lower amount of said at least one metabolite in said at least one tissue.

Embodiment 10. The method of any one of embodiments 1 to 9, wherein said NUE comprises decreased NUE as compared to a tobacco line that comprises an equal amount of said at least one metabolite in said at least one tissue.

Embodiment 11. The method of any one of embodiments 1 to 10, wherein said NUE comprises decreased NUE as compared to a tobacco line that comprises an equal amount of said at least one metabolite in said at least one tissue.

Embodiment 12. The method of any one of embodiments 1 to 11, wherein said determining the amount of said at least one metabolite comprises a method selected from the group consisting of liquid chromatography/mass spectrometry (LC/MS), high-performance liquid chromatography (HPLC), ultra HPLC (UHPLC), mass spectrometry (MS), tandem mass spectrometry (MS/MS), matrix assisted laser desorption/ionization mass spectrometry (MALDI-MS), X-ray fluorescence spectrometry (XRF), ion chromatography (IC), gas chromatography (GC), gas chromatography/mass spectrometry (GC/MS), capillary electrophoresis/mass spectrometry (CE-MS), ion mobility spectrometry/mass spectrometry (IMS/MS), X-ray diffraction, nuclear magnetic resonance (NMR), emission spectral analysis, polarography, ultraviolet-visual spectrometry, infrared spectrometry, thin-layer chromatography.

Embodiment 13. The method of any one of embodiments 1 to 12, wherein said at least one metabolite comprises at least two metabolites.

Embodiment 14. The method of any one of embodiments 1 to 13, wherein said at least one metabolite comprises at least five metabolites.

Embodiment 15. The method of any one of embodiments 1 to 14, wherein said at least one metabolite comprises at least ten metabolites.

Embodiment 16. A method of determining the nitrogen utilization efficiency (NUE) of a tobacco line using a metabolite signature comprising:
a. isolating said metabolite signature from a tobacco plant of said tobacco line;
b. determining the amount of each metabolite comprising said metabolite signature;
c. determining said NUE of said tobacco line by comparing said metabolite signature to a control metabolite signature from a control tobacco line comprising a known NUE.

Embodiment 17. The method of embodiment 16, wherein said metabolite signature comprises at least 2, at least 3, at least 4, at least 5, at least 6, at least 7, at least 8, at least 9, at least 10, at least 11, at least 12, at least 13, at least 14, at least 15, at least 20, at least 25, at least 30, at least 35, at least 40, at least 45, or at least 50 metabolites.

Embodiment 18. The method of embodiments 16 or 17, wherein said NUE comprises enhanced NUE as compared to said control tobacco line.

Embodiment 19. The method of any one of embodiments 16 to 18, wherein said NUE comprises reduced NUE as compared to said control tobacco line.

Embodiment 20. The method of any one of embodiments 16 to 19, wherein said metabolite signature is isolated from a plant tissue selected from the group consisting of root tissue, leaf tissue, floral tissue, meristem tissue, and stem tissue.

Embodiment 21. A method of breeding a tobacco line comprising a metabolite signature associated with enhanced nitrogen utilization efficiency (NUE) comprising:
a. determining the metabolite signature of a first tobacco plant from a first tobacco line, wherein said first tobacco plant comprises enhanced NUE as compared to a control tobacco plant lacking said metabolite signature;
b. crossing said first plant with a second plant of a second tobacco line; and
c. obtaining at least one progeny seed from the crossing of step (a), wherein a progeny plant grown from said at least one progeny seed comprises said metabolite signature, and wherein said progeny plant comprises enhanced NUE as compared to a control plant lacking said metabolite signature.

Embodiment 22. The method of embodiment 21, wherein said method further comprises:
d. crossing said progeny plant to a tobacco plant from said first tobacco line.

Embodiment 23. The method of embodiments 21 or 22, wherein said first tobacco line is selected from the group consisting of MD609, MD601, Banket A1, K326, K346, K358, K394, K399, K730, NC196, NC37NF, NC471, NC55, NC92, NC2326, NC95, NC925.

Embodiment 24. The method of any one of embodiments 21 to 23, wherein said second tobacco line is selected from the group consisting of TN86, TN86LC, TN90, TN90LC, TN97, TN97LC.

Embodiment 25. The method of any one of embodiments 21 to 24, wherein said metabolite signature comprises a leaf metabolite signature.

Embodiment 26. The method of any one of embodiments 21 to 25, wherein said metabolite signature comprises a root metabolite signature.

Embodiment 27. The method of any one of embodiments 21 to 26, wherein said enhanced NUE comprises an increased partial factor productivity (PFP) compared to a tobacco plant lacking said enhanced NUE grown in the same conditions.

Embodiment 28. The method of any one of embodiments 21 to 27, wherein said enhanced NUE comprises an increased agronomic efficiency (AE) compared to a tobacco plant lacking said enhanced NUE grown in the same conditions.

Embodiment 29. The method of any one of embodiments 21 to 28, wherein said enhanced NUE comprises an increased recovery efficiency (RE) compared to a tobacco plant lacking said enhanced NUE grown in the same conditions.

Embodiment 30. The method of any one of embodiments 21 to 29, wherein said enhanced NUE comprises an increased physiological efficiency (PE) compared to a tobacco plant lacking said enhanced NUE grown in the same conditions.

Embodiment 31. The method of any one of embodiments 21 to 30, wherein said enhanced NUE comprises an increased internal efficiency (IE) compared to a tobacco plant lacking said enhanced NUE grown in the same conditions.

Embodiment 32. The method of any one of embodiments 21 to 31, wherein said metabolite signature comprises equal amounts of 4-guanidinobutanoate, syringaldehyde, thiamin, p-hydroxybenzaldehyde, X-23454, X-23580, X-23852, or any combination thereof as compared to the metabolite signature of said control tobacco plant.

Embodiment 33. The method of any one of embodiments 21 to 32, wherein said metabolite signature comprises lower amounts of X-2357, N-acetylmuramate, X-23319, X-23852, X-23330, alpha-ketoglutarate, X-21756, 4-hydroxy-2-oxoglutaric acid, X-23937, X-23916, 1-methyladenine, X-23453, X-11429, X-21796, N′-methylnicotinamide, cotinine, X-23389, N-acetylarginine, N-23366, N-acetylphenylalanine, naringenin, or any combination thereof as compared to the metabolite signature of said control tobacco plant.

Embodiment 34. The method of any one of embodiments 21 to 33, wherein said metabolite signature comprises at least 2, at least 3, at least 4, at least 5, at least 6, at least 7, at least 8, at least 9, at least 10, at least 11, at least 12, at least 13, at least 14, at least 15, at least 20, at least 25, at least 30, at least 35, at least 40, at least 45, or at least 50 metabolites.

Embodiment 35. A method of selecting a tobacco plant comprising:
a. obtaining a population of tobacco plants;
b. isolating at least one metabolite associated with enhanced nitrogen utilization efficiency (NUE) from at least one tobacco plant of said population of tobacco plants;
c. selecting at least one tobacco plant that comprises a equal amount of said at least one metabolite as compared to a control tobacco plant.

Embodiment 36. The method of embodiment 35, wherein said tobacco plant selected in step (c) comprises a equal NUE as compared to said control tobacco plant.

Embodiment 37. The method of embodiment 35 or 36, wherein said enhanced NUE comprises an increased partial factor productivity (PFP) compared to a tobacco plant lacking said enhanced NUE grown in the same conditions.

Embodiment 38. The method of any one of embodiments 35 to 37, wherein said enhanced NUE comprises an increased agronomic efficiency (AE) compared to a tobacco plant lacking said enhanced NUE grown in the same conditions.

Embodiment 39. The method of any one of embodiments 35 to 38, wherein said enhanced NUE comprises an increased recovery efficiency (RE) compared to a tobacco plant lacking said enhanced NUE grown in the same conditions.

Embodiment 40. The method of any one of embodiments 35 to 39, wherein said enhanced NUE comprises an increased physiological efficiency (PE) compared to a tobacco plant lacking said enhanced NUE grown in the same conditions.

Embodiment 41. The method of any one of embodiments 35 to 40, wherein said enhanced NUE comprises an increased internal efficiency (IE) compared to a tobacco plant lacking said enhanced NUE grown in the same conditions.

Embodiment 42. The method of any one of embodiments 35 to 41, wherein said at least one metabolite is selected from the group consisting of 4-guanidinobutanoate, syringaldehyde, thiamin, p-hydroxybenzaldehyde, X-23454, X-23580, X-23852, or any combination thereof.

Embodiment 43. The method of any one of embodiments 35 to 42, wherein said at least one metabolite is isolated from leaf tissue or root tissue.

Embodiment 44. The method of any one of embodiments 35 to 43, wherein said at least one metabolite is isolated from a plant tissue selected from the group consisting of root tissue, leaf tissue, floral tissue, meristem tissue, and stem tissue.

Embodiment 45. A method of selecting a tobacco plant comprising:
a. obtaining a population of tobacco plants;
b. isolating at least one metabolite associated with enhanced nitrogen utilization efficiency (NUE) from at least one tobacco plant of said population of tobacco plants;
c. selecting at least one tobacco plant that comprises a lower amount of said at least one metabolite as compared to a control tobacco plant.

Embodiment 46. The method of embodiment 45, wherein said tobacco plant selected in step (c) comprises a equal NUE as compared to said control tobacco plant.

Embodiment 47. The method of embodiment 45 or 46, wherein said enhanced NUE comprises an increased partial factor productivity (PFP) compared to a tobacco plant lacking said enhanced NUE grown in the same conditions.

Embodiment 48. The method of any one of embodiments 45 to 47, wherein said enhanced NUE comprises an increased agronomic efficiency (AE) compared to a tobacco plant lacking said enhanced NUE grown in the same conditions.

Embodiment 49. The method of any one of embodiments 45 to 48, wherein said enhanced NUE comprises an increased recovery efficiency (RE) compared to a tobacco plant lacking said enhanced NUE grown in the same conditions.

Embodiment 50. The method of any one of embodiments 45 to 49, wherein said enhanced NUE comprises an increased physiological efficiency (PE) compared to a tobacco plant lacking said enhanced NUE grown in the same conditions.

Embodiment 51. The method of any one of embodiments 45 to 50, wherein said enhanced NUE comprises an increased internal efficiency (IE) compared to a tobacco plant lacking said enhanced NUE grown in the same conditions.

Embodiment 52. The method of any one of embodiments 45 to 51, wherein said at least one metabolite is selected from the group consisting of X-2357, N-acetylmuramate, X-23319, X-23852, X-23330, alpha-ketoglutarate, X-21756, 4-hydroxy-2-oxoglutaric acid, X-23937, X-23916, 1-methyladenine, X-23453, X-11429, X-21796, N′-methylnicotinamide, cotinine, X-23389, N-acetylarginine, N-23366, N-acetylphenylalanine, naringenin, or any combination thereof.

Embodiment 53. The method of any one of embodiments 45 to 52, wherein said at least one metabolite is isolated from leaf tissue or root tissue.

Embodiment 54. The method of any one of embodiments 45 to 53, wherein said at least one metabolite is isolated from a plant tissue selected from the group consisting of root tissue, leaf tissue, floral tissue, meristem tissue, and stem tissue.

Embodiment 55. A method of screening a tobacco plant for a metabolite signature associated with enhanced nitrogen utilization efficiency (NUE) comprising:
a. isolating a first metabolite signature associated with enhanced NUE from said tobacco plant;
b. determining the amount of at least one metabolite comprising said first metabolite signature;
c. comparing said first metabolite signature to a second metabolite signature of a control tobacco plant that comprises a known NUE; and
d. determining if said first metabolite signature is associated with enhanced NUE.

Embodiment 56. The method of embodiment 55, wherein said enhanced NUE comprises an increased partial factor productivity (PFP) compared to a tobacco plant lacking said enhanced NUE grown in the same conditions.

Embodiment 57. The method of embodiment 55 or 56, wherein said enhanced NUE comprises an increased agronomic efficiency (AE) compared to a tobacco plant lacking said enhanced NUE grown in the same conditions.

Embodiment 58. The method of any one of embodiments 55 to 57, wherein said enhanced NUE comprises an increased recovery efficiency (RE) compared to a tobacco plant lacking said enhanced NUE grown in the same conditions.

Embodiment 59. The method of any one of embodiments 55 to 58, wherein said enhanced NUE comprises an increased physiological efficiency (PE) compared to a tobacco plant lacking said enhanced NUE grown in the same conditions.

Embodiment 60. The method of any one of embodiments 55 to 59, wherein said enhanced NUE comprises an increased internal efficiency (IE) compared to a tobacco plant lacking said enhanced NUE grown in the same conditions.

Embodiment 61. A modified tobacco seed, or tobacco plant grown therefrom, comprising a cisgenic polynucleotide comprising a heterologous promoter operably linked to a coding region, wherein said modified tobacco plant comprises enhanced nitrogen utilization efficiency as compared to an unmodified control tobacco plant lacking said cisgenic polynucleotide when grown under the same conditions.

Embodiment 62. The modified tobacco seed or tobacco plant of embodiment 61, wherein said coding region encodes a polypeptide at least 70% identical or similar to a sequence selected from the group consisting of SEQ ID NOs:1 to 8.

Embodiment 63. The modified tobacco seed or tobacco plant of embodiment 61 or 62, wherein said coding region comprises a polynucleotide sequence at least 70% identical or complementary to a polynucleotide sequence selected from the group consisting of SEQ ID NOs:9 to 16.

Embodiment 64. The modified tobacco seed or tobacco plant of any one of embodiments 61 to 63, wherein said heterologous promoter is selected from the group consisting of a constitutive promoter, an inducible promoter, a tissue-preferred promoter, and a tissue-specific promoter.

Embodiment 65. The modified tobacco seed or tobacco plant of any one of embodiments 61 to 64, wherein said heterologous promoter comprises a polynucleotide sequence from a tobacco genome.

Embodiment 66. The modified tobacco seed or tobacco plant of any one of embodiments 61 to 65, wherein said heterologous promoter comprises a polynucleotide sequence from a plant genome.

Embodiment 67. The modified tobacco seed or tobacco plant of any one of embodiments 61 to 66, wherein said tissue-preferred promoter is a leaf-preferred promoter.

Embodiment 68. The modified tobacco seed or tobacco plant of any one of embodiments 61 to 67, wherein said tissue-preferred promoter is a root-preferred promoter.

Embodiment 69. The modified tobacco seed or tobacco plant of any one of embodiments 61 to 68, wherein said leaf-preferred promoter is encoded by a sequence at least 70% identical or complementary to a sequence selected from the group consisting of SEQ ID NOs:17 to 19, or a functional fragment thereof.

Embodiment 70. The modified tobacco seed or tobacco plant of any one of embodiments 61 to 69, wherein said root-preferred promoter is encoded by a sequence at least 70% identical or complementary to a sequence selected from the group consisting of SEQ ID NOs:20 to 24, or a functional fragment thereof.

Embodiment 71. The modified tobacco plant of any one of embodiments 61 to 70, wherein said modified tobacco plant comprises equal levels of a metabolite selected from the group consisting of 4-guanidinobutanoate, syringaldehyde, thiamin, and p-hydroxybenzaldehyde in root tissue as compared to an unmodified tobacco plant lacking said cisgenic polynucleotide when grown under the same conditions.

Embodiment 72. The modified tobacco plant of any one of embodiments 61 to 71, wherein said modified tobacco plant comprises equal levels of a metabolite selected from the group consisting of 4-guanidinobutanoate, X-23454, X-23580, and X-23852 in leaf tissue as compared to an unmodified tobacco plant lacking said cisgenic polynucleotide when grown under the same conditions.

Embodiment 73. The modified tobacco plant of any one of embodiments 61 to 72, wherein said modified tobacco plant comprises lower levels of a metabolite selected from the group consisting of X-2357, N-acetylmuramate, X-23319, X-23852, X-23330, alpha-ketoglutarate, X-21756, 4-hydroxy-2-oxoglutaric acid, X-23937, X-23916, and 1-methyladenine in root tissue as compared to an unmodified tobacco plant lacking said cisgenic polynucleotide when grown under the same conditions.

Embodiment 74. The modified tobacco plant of any one of embodiments 61 to 73, wherein said modified tobacco plant comprises lower levels of a metabolite selected from the group consisting of X-23453, X-21756, X-11429, X-21796, N′-methylnicotinamide, cotinine, X-23389, N-acetylarginine, N-23366, N-acetylphenylalanine, and naringenin in leaf tissue as compared to an unmodified tobacco plant lacking said cisgenic polynucleotide when grown under the same conditions.

Embodiment 75. The modified tobacco seed or plant of any one of embodiments 61 to 74, wherein said modified tobacco seed or plant is of a Burley variety.

Embodiment 76. The modified tobacco seed or plant of any one of embodiments 61 to 75, wherein said modified tobacco seed or plant comprises lower amounts of TSNAs as compared to an unmodified tobacco plant lacking said cisgenic polynucleotide when grown under the same conditions.

Embodiment 77. A recombinant DNA construct comprising a heterologous promoter operably linked to a polynucleotide encoding a polypeptide at least 70% identical or similar to a polypeptide selected from the group consisting of SEQ ID NOs:1 to 8.

Embodiment 78. Cured tobacco material, or a tobacco product comprising said cured tobacco material, wherein said cured tobacco material is made from a tobacco plant comprising a cisgenic polynucleotide comprising a heterologous promoter operably linked to a coding region, wherein said modified tobacco plant comprises enhanced nitrogen utilization efficiency as compared to an unmodified control tobacco plant lacking said cisgenic polynucleotide when grown under the same conditions.

Embodiment 79. A greenhouse, growth chamber, or field comprising the modified tobacco seed or plant of any one of embodiments 61 to 76.

Embodiment 80. A modified tobacco seed, or tobacco plant grown therefrom, comprising at least one mutation in an endogenous locus encoding a polypeptide selected from the group consisting of SEQ ID NOs:25 to 40, and wherein said modified tobacco seed or tobacco plant comprises enhanced nitrogen utilization efficiency as compared to an unmodified control tobacco plant lacking said at least one mutation when grown under the same conditions.

Embodiment 81. The modified tobacco seed or tobacco plant of embodiment 80, wherein said at least one mutation is selected from the group consisting of an insertion, a deletion, a substitution, and an inversion.

Embodiment 82. The modified tobacco seed or tobacco plant of embodiment 80 or 81, wherein said at least one mutation is a null mutation.

Embodiment 83. The modified tobacco plant of any one of embodiments 80 to 82, wherein said modified tobacco plant comprises equal levels of a metabolite selected from the group consisting of 4-guanidinobutanoate, syringaldehyde, thiamin, and p-hydroxybenzaldehyde in root tissue as compared to an unmodified tobacco plant lacking said at least one mutation when grown under the same conditions.

Embodiment 84. The modified tobacco plant of any one of embodiments 80 to 83, wherein said modified tobacco plant comprises equal levels of a metabolite selected from the group consisting of 4-guanidinobutanoate, X-23454, X-23580, and X-23852 in leaf tissue as compared to an unmodified tobacco plant lacking said at least one mutation when grown under the same conditions.

Embodiment 85. The modified tobacco plant of any one of embodiments 80 to 84, wherein said modified tobacco plant comprises lower levels of a metabolite selected from the group consisting of X-2357, N-acetylmuramate, X-23319, X-23852, X-23330, alpha-ketoglutarate, X-21756, 4-hydroxy-2-oxoglutaric acid, X-23937, X-23916, and 1-methyladenine in root tissue as compared to an unmodified tobacco plant lacking said at least one mutation when grown under the same conditions.

Embodiment 86. The modified tobacco plant of any one of embodiments 80 to 85, wherein said modified tobacco plant comprises lower levels of a metabolite selected from the group consisting of X-23453, X-21756, X-11429, X-21796, N′-methylnicotinamide, cotinine, X-23389, N-acetylarginine, N-23366, N-acetylphenylalanine, and naringenin in leaf tissue as compared to an unmodified tobacco plant lacking said at least one mutation when grown under the same conditions.

Embodiment 87. The modified tobacco seed or plant of any one of embodiments 80 to 86, wherein said modified tobacco seed or plant is of a Burley variety.

Embodiment 88. The modified tobacco seed or plant of any one of embodiments 80 to 87, wherein said modified tobacco seed or plant comprises lower amounts of TSNAs as compared to an unmodified tobacco plant lacking said at least one mutation when grown under the same conditions.

Embodiment 89. A recombinant DNA construct comprising a heterologous promoter operably linked to a guide RNA comprising at least 18, at least 19, at least 20, at least 21, at least 22, at least 23, at least 24, at least 25, at least 26, at least 27, or at least 28 contiguous nucleotides identical or complementary to a polynucleotide encoding a polypeptide selected from the group consisting of SEQ ID NOs:25 to 40.

Embodiment 90. Cured tobacco material, or a tobacco product comprising said cured tobacco material, wherein said cured tobacco material is made from a tobacco plant comprising at least one mutation in an endogenous locus encoding a polypeptide selected from the group consisting of SEQ ID NOs:25 to 40, and wherein said modified tobacco seed or tobacco plant comprises enhanced nitrogen utilization efficiency as compared to an unmodified control tobacco plant lacking said at least one mutation when grown under the same conditions.

Embodiment 91. A modified tobacco seed, or tobacco plant grown therefrom, comprising a cisgenic polynucleotide comprising a heterologous promoter operably linked to a polynucleotide encoding a small RNA (sRNA) at least 85% identical or complementary to a polynucleotide selected from the group consisting of SEQ ID NOs:41 to 56, and wherein said modified tobacco seed or tobacco plant comprises enhanced nitrogen utilization efficiency as compared to an unmodified control tobacco plant lacking said cisgenic polynucleotide when grown under the same conditions.

Embodiment 92. The modified tobacco seed or tobacco plant of embodiment 91, wherein said sRNA comprises at least 18, at least 19, at least 20, at least 21, at least 22, at least 23, at least 24, at least 25, at least 26, at least 27, or at least 28 nucleotides.

Embodiment 93. The modified tobacco seed or tobacco plant of embodiment 91 or 92, wherein said sRNA is selected from the group consisting of a microRNA, a small-interfering RNA (siRNA), a trans-acting siRNA, and precursors thereof.

Embodiment 94. The modified tobacco seed or tobacco plant of any one of embodiments 91 to 93, wherein said sRNA down-regulates the expression or translation of a polynucleotide selected from the group consisting of SEQ ID NOs:41 to 56.

Embodiment 95. The modified tobacco seed or tobacco plant of any one of embodiments 91 to 94, wherein said heterologous promoter is selected from the group consisting of a constitutive promoter, an inducible promoter, a tissue-preferred promoter, and a tissue-specific promoter.

Embodiment 96. The modified tobacco seed or tobacco plant of any one of embodiments 91 to 95, wherein said tissue-preferred promoter is a leaf-preferred promoter.

Embodiment 97. The modified tobacco seed or tobacco plant of any one of embodiments 91 to 96, wherein said tissue-preferred promoter is a root-preferred promoter.

Embodiment 98. The modified tobacco seed or tobacco plant of any one of embodiments 91 to 97, wherein said leaf-preferred promoter is encoded by a sequence at least 70% identical or complementary to a sequence selected from the group consisting of SEQ ID NOs:17 to 19, or a functional fragment thereof.

Embodiment 99. The modified tobacco seed or tobacco plant of any one of embodiments 91 to 98, wherein said root-preferred promoter is encoded by a sequence at least 70% identical or complementary to a sequence selected from the group consisting of SEQ ID NOs:20 to 24, or a functional fragment thereof.

Embodiment 100. The modified tobacco seed or tobacco plant of any one of embodiments 91 to 99, wherein said heterologous promoter comprises a polynucleotide sequence from a tobacco genome.

Embodiment 101. The modified tobacco seed or tobacco plant of any one of embodiments 91 to 100, wherein said heterologous promoter comprises a polynucleotide sequence from a plant genome.

Embodiment 102. The modified tobacco plant of any one of embodiments 91 to 101, wherein said modified tobacco plant comprises equal levels of a metabolite selected from the group consisting of 4-guanidinobutanoate, syringaldehyde, thiamin, and p-hydroxybenzaldehyde in root tissue as compared to an unmodified tobacco plant lacking said cisgenic polynucleotide when grown under the same conditions.

Embodiment 103. The modified tobacco plant of any one of embodiments 91 to 102, wherein said modified tobacco plant comprises equal levels of a metabolite selected from the group consisting of 4-guanidinobutanoate, X-23454, X-23580, and X-23852 in leaf tissue as compared to an unmodified tobacco plant lacking said cisgenic polynucleotide when grown under the same conditions.

Embodiment 104. The modified tobacco plant of any one of embodiments 91 to 103, wherein said modified tobacco plant comprises lower levels of a metabolite selected from the group consisting of X-2357, N-acetylmuramate, X-23319, X-23852, X-23330, alpha-ketoglutarate, X-21756, 4-hydroxy-2-oxoglutaric acid, X-23937, X-23916, and 1-methyladenine in root tissue as compared to an unmodified tobacco plant lacking said cisgenic polynucleotide when grown under the same conditions.

Embodiment 105. The modified tobacco plant of any one of embodiments 91 to 104, wherein said modified tobacco plant comprises lower levels of a metabolite selected from the group consisting of X-23453, X-21756, X-11429, X-21796, N′-methylnicotinamide, cotinine, X-23389, N-acetylarginine, N-23366, N-acetylphenylalanine, and naringenin in leaf tissue as compared to an unmodified tobacco plant lacking said cisgenic polynucleotide when grown under the same conditions.

Embodiment 106. The modified tobacco seed or plant of any one of embodiments 91 to 105, wherein said modified tobacco seed or plant is of a Burley variety.

Embodiment 107. The modified tobacco seed or plant of any one of embodiments 91 to 106, wherein said modified tobacco seed or plant comprises lower amounts of TSNAs as compared to an unmodified tobacco plant lacking said cisgenic polynucleotide when grown under the same conditions.

Embodiment 108. A recombinant DNA construct comprising a heterologous promoter operably linked to a polynucleotide encoding a small RNA (sRNA) at least 85% identical or complementary to a polynucleotide selected from the group consisting of SEQ ID NOs:41 to 56.

Embodiment 109. Cured tobacco material, or a tobacco product comprising said cured tobacco material, wherein said cured tobacco material is made from a tobacco plant comprising a cisgenic polynucleotide comprising a heterologous promoter operably linked to a polynucleotide encoding a small RNA (sRNA) at least 85% identical or complementary to a polynucleotide selected from the group consisting of SEQ ID NOs:41 to 56, and wherein said modified tobacco seed or tobacco plant comprises enhanced nitrogen utilization efficiency as compared to an unmodified control tobacco plant lacking said cisgenic polynucleotide when grown under the same conditions.

Embodiment 110. A method of enhancing the nitrogen utilization efficiency (NUE) of a tobacco plant comprising:
a. introducing a cisgenic nucleic acid molecule into a tobacco cell; and
b. regenerating a modified tobacco plant from said tobacco cell, wherein said modified tobacco plant comprises enhanced NUE as compared to a tobacco plant lacking said cisgenic nucleic acid molecule.

Embodiment 111. The method of embodiment 110, wherein said method further comprises:
c. crossing said modified tobacco plant with a second tobacco plant or self-pollinating said modified tobacco plant.

Embodiment 112. The method of embodiment 110 or 111, wherein said enhanced NUE comprises an increased partial factor productivity (PFP) compared to a tobacco plant lacking said enhanced NUE grown in the same conditions.

Embodiment 113. The method of any one of embodiments 110 to 112, wherein said enhanced NUE comprises an increased agronomic efficiency (AE) compared to a tobacco plant lacking said enhanced NUE grown in the same conditions.

Embodiment 114. The method of any one of embodiments 110 to 113, wherein said enhanced NUE comprises an increased recovery efficiency (RE) compared to a tobacco plant lacking said enhanced NUE grown in the same conditions.

Embodiment 115. The method of any one of embodiments 110 to 114, wherein said enhanced NUE comprises an increased physiological efficiency (PE) compared to a tobacco plant lacking said enhanced NUE grown in the same conditions.

Embodiment 116. The method of any one of embodiments 110 to 115, wherein said enhanced NUE comprises an increased internal efficiency (IE) compared to a tobacco plant lacking said enhanced NUE grown in the same conditions.

Embodiment 117. A method of enhancing the nitrogen utilization efficiency (NUE) of a tobacco plant comprising:
a. introducing a modification to a nucleic acid molecule encoding a gene having sequence selected from the group consisting of SEQ ID NOs:41 to 56 in a tobacco cell;
b. regenerating a modified tobacco plant from said tobacco cell, wherein said modified tobacco plant comprises enhanced NUE as compared to a tobacco plant lacking said modification.

Embodiment 118. The method of embodiment 117, wherein said introduction comprises the use of an RNA-guided nuclease.

Embodiment 119. The method of embodiment 117 or 118, wherein said RNA-guided nuclease is selected from the group consisting of a Cas9 nuclease, a Cpf1 nuclease, a CasX nuclease, a CasY nuclease, and functional homologues thereof.

Embodiment 120. The method of any one of embodiments 117 to 119, wherein said modification is selected from the group consisting of an insertion, a substitution, an inversion, and a deletion.

Embodiment 121. The method of any one of embodiments 117 to 120, wherein said enhanced NUE comprises an increased partial factor productivity (PFP) compared to a tobacco plant lacking said enhanced NUE grown in the same conditions.

Embodiment 122. The method of any one of embodiments 117 to 121, wherein said enhanced NUE comprises an increased agronomic efficiency (AE) compared to a tobacco plant lacking said enhanced NUE grown in the same conditions.

Embodiment 123. The method of any one of embodiments 117 to 122, wherein said enhanced NUE comprises an increased recovery efficiency (RE) compared to a tobacco plant lacking said enhanced NUE grown in the same conditions.

Embodiment 124. The method of any one of embodiments 117 to 123, wherein said enhanced NUE comprises an increased physiological efficiency (PE) compared to a tobacco plant lacking said enhanced NUE grown in the same conditions.

Embodiment 125. The method of any one of embodiments 117 to 124, wherein said enhanced NUE comprises an increased internal efficiency (IE) compared to a tobacco plant lacking said enhanced NUE grown in the same conditions.

Embodiment 126. The method of any one of embodiments 117 to 125, wherein said method further comprises:
c. crossing said modified tobacco plant with a second tobacco plant or self-pollinating said modified tobacco plant.

Embodiment 127. A method of enhancing the nitrogen utilization efficiency (NUE) of a tobacco plant comprising:
a. introducing a nucleic acid encoding a small RNA (sRNA) homologous to at least 18 contiguous nucleic acids of a nucleic acid molecule encoding a gene having sequence selected from the group consisting of SEQ ID NOs:41 to 56 in a tobacco cell;
b. regenerating a modified tobacco plant from said tobacco cell, wherein said modified tobacco plant comprises enhanced NUE as compared to a tobacco plant lacking said sRNA.

Embodiment 128. The method of embodiment 127, wherein said sRNA is selected from the group consisting of a microRNA, a small-interfering RNA (siRNA), a trans-acting siRNA, and precursors thereof.

Embodiment 129. The method of embodiment 127 or 128, wherein said enhanced NUE comprises an increased partial factor productivity (PFP) compared to a tobacco plant lacking said enhanced NUE grown in the same conditions.

Embodiment 130. The method of any one of embodiments 127 to 129, wherein said enhanced NUE comprises an increased agronomic efficiency (AE) compared to a tobacco plant lacking said enhanced NUE grown in the same conditions.

Embodiment 131. The method of any one of embodiments 127 to 130, wherein said enhanced NUE comprises an increased recovery efficiency (RE) compared to a tobacco plant lacking said enhanced NUE grown in the same conditions.

Embodiment 132. The method of any one of embodiments 127 to 131, wherein said enhanced NUE comprises an increased physiological efficiency (PE) compared to a tobacco plant lacking said enhanced NUE grown in the same conditions.

Embodiment 133. The method of any one of embodiments 127 to 132, wherein said enhanced NUE comprises an increased internal efficiency (IE) compared to a tobacco plant lacking said enhanced NUE grown in the same conditions.

Embodiment 134. The method of any one of embodiments 127 to 133, wherein said method further comprises:
c. crossing said modified tobacco plant with a second tobacco plant or self-pollinating said modified tobacco plant.

Embodiment 135. A method comprising:
a. providing a first population of tobacco plants comprising enhanced nitrogen utilization efficiency;
b. genotyping said first population of tobacco plants for the presence of a molecular marker within 20 cM of an enhanced nitrogen utilization efficiency locus; and
c. selecting one or more tobacco plants genotyped in step (b) that comprise said molecular marker.

Embodiment 136. The method of embodiment 135, wherein said method further comprises:
d. crossing one or more tobacco plants selected in step (c) to a second tobacco plant; and
e. obtaining progeny seed from the cross of step (d).

Embodiment 137. The method of embodiment 135 or 136, wherein said molecular marker is selected from the group consisting of a SNP marker, an INDEL marker, an RFLP marker, an SSR marker, an AFLP marker, and a RAPD marker.

Embodiment 138. The method of any one of embodiments 135 to 137, wherein said enhanced nitrogen utilization efficiency locus comprises a polynucleotide encoding a polypeptide at least 70% identical or similar to a polypeptide selected from the group consisting of SEQ ID NOs:1 to 8.

Embodiment 139. The method of any one of embodiments 135 to 138, wherein said enhanced nitrogen utilization efficiency locus comprises a polynucleotide sequence selected from the group consisting of SEQ ID NOs:9 to 16.

Embodiment 140. The method of any one of embodiments 135 to 139, wherein said molecular marker is selected from the group consisting of SEQ ID NOs:57 to 64.

Embodiment 141. The method of any one of embodiments 135 to 140, wherein said molecular marker comprises a G nucleotide at position 57 of SEQ ID NO:58.

Embodiment 142. The method of any one of embodiments 135 to 141, wherein said molecular marker comprises a C nucleotide at position 117 of SEQ ID NO:58.

Embodiment 143. The method of any one of embodiments 135 to 142, wherein said molecular marker comprises a G nucleotide at position 57 and a C nucleotide at position 117 of SEQ ID NO:58.

Embodiment 144. The method of any one of embodiments 135 to 143, wherein said molecular marker comprises a T nucleotide at position 14 of SEQ ID NO:57.

Embodiment 145. The method of any one of embodiments 135 to 144, wherein said molecular marker comprises a G nucleotide at position 162 of SEQ ID NO:59.

Embodiment 146. The method of any one of embodiments 135 to 145, wherein said molecular marker comprises a C nucleotide at position 36 of SEQ ID NO:60.

Embodiment 147. The method of any one of embodiments 135 to 146, wherein said molecular marker comprises a T nucleotide at position 36 of SEQ ID NO:61.

Embodiment 148. The method of any one of embodiments 135 to 147, wherein said molecular marker comprises a T nucleotide at position 36 of SEQ ID NO:62.

Embodiment 149. The method of any one of embodiments 135 to 148, wherein said molecular marker comprises a G nucleotide at position 36 of SEQ ID NO:63.

Embodiment 150. The method of any one of embodiments 135 to 149, wherein said molecular marker comprises a T nucleotide at position 36 of SEQ ID NO:64.

Embodiment 151. The method of any one of embodiments 135 to 150, wherein said first population of tobacco plants is of a Maryland variety.

Embodiment 152. The method of any one of embodiments 135 to 151, wherein said first population of tobacco plants is of a variety selected from the group consisting of MD609, MD601, Banket A1, K326, K346, K358, K394, K399, K730, NC196, NC37NF, NC471, NC55, NC92, NC2326, NC95, NC925.

Embodiment 153. The method of any one of embodiments 135 to 152, wherein said second tobacco plant is of a variety selected from the group consisting of TN86, TN86LC, TN90, TN90LC, TN97, TN97LC.

Embodiment 154. The method of any one of embodiments 135 to 153, wherein said progeny seed comprises said molecular marker.

Embodiment 155. The method of any one of embodiments 135 to 154, wherein said progeny seed comprises said enhanced nitrogen utilization efficiency.

Embodiment 156. The method of any one of embodiments 135 to 155, wherein said molecular marker is within 15 cM of said enhanced nitrogen utilization efficiency locus.

Embodiment 157. The method of any one of embodiments 135 to 156, wherein said molecular marker is within 10 cM of said enhanced nitrogen utilization efficiency locus.

Embodiment 158. The method of any one of embodiments 135 to 157, wherein said molecular marker is within 5 cM of said enhanced nitrogen utilization efficiency locus.

Embodiment 159. The method of any one of embodiments 135 to 158, wherein said molecular marker is within 2 cM of said enhanced nitrogen utilization efficiency locus.

Embodiment 160. The method of any one of embodiments 135 to 159, wherein said molecular marker is within 1 cM of said enhanced nitrogen utilization efficiency locus.

Embodiment 161. The method of any one of embodiments 135 to 160, wherein said molecular marker is within 0.5 cM of said enhanced nitrogen utilization efficiency locus.

Embodiment 162. A method comprising:
a. providing a first population of tobacco plants;
b. genotyping said first population of tobacco plants for the presence of an enhanced nitrogen utilization efficiency allele of a locus encoded by a sequence selected from the group consisting of SEQ ID NOs:9 to 16; and
c. selecting one or more tobacco plants genotyped in step (b) that comprise said enhanced nitrogen utilization efficiency allele.

Embodiment 163. The method of embodiment 161 or 162, wherein said method further comprises:
d. crossing the one or more tobacco plants selected in step (c) to a second tobacco plant; and
e. obtaining progeny seed from the crossing of step (d).

Embodiment 164. The method of any one of embodiments 161 to 163, wherein said first population of tobacco plants is of a Maryland variety.

Embodiment 165. The method of any one of embodiments 161 to 164, wherein said first population of tobacco plants is of a variety selected from the group consisting of MD609, MD601, Banket A1, K326, K346, K358, K394, K399, K730, NC196, NC37NF, NC471, NC55, NC92, NC2326, NC95, NC925.

Embodiment 166. The method of any one of embodiments 161 to 165, wherein said second tobacco plant is of a variety selected from the group consisting of TN86, TN86LC, TN90, TN90LC, TN97, TN97LC.

Embodiment 167. The method of any one of embodiments 161 to 166, wherein said progeny seed comprises said molecular marker.

Embodiment 168. The method of any one of embodiments 161 to 167, wherein said progeny seed comprises said enhanced nitrogen utilization efficiency.

Embodiment 169. A method of introgressing an enhanced nitrogen utilization efficiency trait into a tobacco variety comprising:
a. crossing a first tobacco variety comprising said enhanced nitrogen utilization efficiency trait with a second tobacco variety lacking said enhanced nitrogen utilization efficiency trait;
b. obtaining progeny seed from the cross of step (a);
c. genotyping at least one of said progeny seed obtained in step (b) for a molecular marker linked to said enhanced nitrogen utilization efficiency trait, wherein said molecular marker is within 20 cM of a locus selected from the group consisting of SEQ ID NOs:9 to 16; and
d. selecting a progeny seed comprising said enhanced nitrogen utilization efficiency trait.

Embodiment 170. The method of embodiment 169, wherein said first tobacco variety is a Maryland tobacco variety.

Embodiment 171. The method of embodiment 169 or 170, wherein said first tobacco variety is selected from the group consisting of MD609, MD601, Banket A1, K326, K346, K358, K394, K399, K730, NC196, NC37NF, NC471, NC55, NC92, NC2326, NC95, NC925.

Embodiment 172. The method of any one of embodiments 169 to 171, wherein said second tobacco variety is a Burley tobacco variety.

Embodiment 173. The method of any one of embodiments 169 to 172, wherein said second tobacco variety is selected from the group consisting of TN86, TN86LC, TN90, TN90LC, TN97, TN97LC.

Embodiment 174. The method of any one of embodiments 169 to 173, wherein said enhanced NUE comprises an increased partial factor productivity (PFP) compared to a tobacco plant lacking said enhanced NUE grown in the same conditions.

Embodiment 175. The method of any one of embodiments 169 to 174, wherein said enhanced NUE comprises an increased agronomic efficiency (AE) compared to a tobacco plant lacking said enhanced NUE grown in the same conditions.

Embodiment 176. The method of any one of embodiments 169 to 175, wherein said enhanced NUE comprises an increased recovery efficiency (RE) compared to a tobacco plant lacking said enhanced NUE grown in the same conditions.

Embodiment 177. The method of any one of embodiments 169 to 176, wherein said enhanced NUE comprises an increased physiological efficiency (PE) compared to a tobacco plant lacking said enhanced NUE grown in the same conditions.

Embodiment 178. The method of any one of embodiments 169 to 177, wherein said enhanced NUE comprises an increased internal efficiency (IE) compared to a tobacco plant lacking said enhanced NUE grown in the same conditions.

Embodiment 179. The method of any one of embodiments 169 to 178, wherein said molecular marker is within 15 cM of said locus.

Embodiment 180. The method of any one of embodiments 169 to 179, wherein said molecular marker is within 10 cM of said locus.

Embodiment 181. The method of any one of embodiments 169 to 180, wherein said molecular marker is within 5 cM of said locus.

Embodiment 182. The method of any one of embodiments 169 to 181, wherein said molecular marker is within 2 cM of said locus.

Embodiment 183. The method of any one of embodiments 169 to 182, wherein said molecular marker is within 1 cM of said locus.

Embodiment 184. The method of any one of embodiments 169 to 183, wherein said molecular marker is within 0.5 cM of said locus.

Embodiment 185. A method of selecting a tobacco plant comprising an enhanced nitrogen utilization efficiency trait comprising:
a. isolating nucleic acids from a collection of tobacco germplasm;
b. assaying said nucleic acids for one or more markers located within 20 cM of a locus selected from the group consisting of SEQ ID NOs:9 to 16; and
c. selecting said tobacco plant comprising said enhanced nitrogen utilization efficiency trait.

Embodiment 186. The method of embodiment 185, wherein said method further comprises:
d. crossing said tobacco plant selected in step (c) with a second tobacco plant; and
e. obtaining progeny seed from the cross of step (d).

Embodiment 187. The method of embodiment 185 or 186, wherein said molecular marker is within 15 cM of said locus.

Embodiment 188. The method of any one of embodiments 185 to 187, wherein said molecular marker is within 10 cM of said locus.

Embodiment 189. The method of any one of embodiments 185 to 188, wherein said molecular marker is within 5 cM of said locus.

Embodiment 190. The method of any one of embodiments 185 to 189, wherein said molecular marker is within 2 cM of said locus.

Embodiment 191. The method of any one of embodiments 185 to 190, wherein said molecular marker is within 1 cM of said locus.

Embodiment 192. The method of any one of embodiments 185 to 191, wherein said molecular marker is within 0.5 cM of said locus.

Embodiment 193. A method of selecting a tobacco plant comprising an enhanced nitrogen utilization efficiency trait comprising:
a. isolating nucleic acids from a collection of tobacco germplasm;
b. assaying said nucleic acids for one or more markers located within 20 cM of SNP marker selected from the group consisting of SEQ ID NOs:57 to 64; and
c. selecting said tobacco plant comprising said enhanced nitrogen utilization efficiency trait.

Embodiment 194. The method of embodiment 193, wherein said assaying comprises assaying for a G nucleotide at position 57 of SEQ ID NO:58.

Embodiment 195. The method of embodiment 193 or 194, wherein said assaying comprises assaying for a C nucleotide at position 117 of SEQ ID NO:58.

Embodiment 196. The method of any one of embodiments 193 to 195, wherein said assaying comprises assaying for a G nucleotide at position 57 and a C nucleotide at position 117 of SEQ ID NO:58.

Embodiment 197. The method of embodiment 193 to 196, wherein said assaying comprises assaying for a T nucleotide at position 14 of SEQ ID NO:57.

Embodiment 198. The method of any one of embodiments 193 to 197, wherein said assaying comprises assaying for a G nucleotide at position 162 of SEQ ID NO:59.

Embodiment 199. The method of any one of embodiments 193 to 198, wherein said assaying comprises assaying for a C nucleotide at position 36 of SEQ ID NO:60.

Embodiment 200. The method of any one of embodiments 193 to 199, wherein said assaying comprises assaying for a T nucleotide at position 36 of SEQ ID NO:61.

Embodiment 201. The method of any one of embodiments 193 to 200, wherein said assaying comprises assaying for a T nucleotide at position 36 of SEQ ID NO:62.

Embodiment 202. The method of any one of embodiments 193 to 201, wherein said assaying comprises assaying for a G nucleotide at position 36 of SEQ ID NO:63.

Embodiment 203. The method of any one of embodiments 193 to 202, wherein said assaying comprises assaying for a T nucleotide at position 36 of SEQ ID NO:64.

### EXAMPLES

### Example 1. Field Production Practices

Field grown tobacco plants are generated using standard field production practices. Each test plot comprises up to 40 rows of transplanted seedlings. Seedlings are germinated in a greenhouse before transplantation. For testing NUE traits, a test plot receives a nitrogen rate of 60 pounds of nitrogen per acre. Plants are topped using standard procedures when 50% of the plants in a test plot reach the elongated button stage. Pesticide application follows standard protocols. Leaves are harvested at maturity and sorted into 3 sticks per plot with 5 plants per stick for curing. Leaves are sampled from the sticks at the takedown/stripping stage. Five leaves are harvested from three different sticks per experimental variety for 15 leaves per sample. Half of the lamina from the fourth leaf from the top of each plant is harvested for sampling. Analytical analysis of alkaloids, TSNA and NO₃ are conducted using routine methods known in the art.

### Example 2. Identification of Metabolites Associated with Enhanced Nitrogen Utilization Efficiency

Maryland tobacco varieties require approximately 25% less nitrogen fertilizer input as compared to Burley tobacco varieties. In order to identify metabolites associated with high nitrogen efficiency (Maryland) and low nitrogen efficiency (Burley) tobacco varieties, differences in metabolite levels were examined in the Maryland tobacco variety MD609 and the Burley tobacco variety TN90.

MD609 and TN90 seedlings were germinated from seed and grown without the addition of nitrogen for six weeks. After six weeks, the seedlings from each variety were split into two groups: Group A comprised plants that were provided with 100 parts per million nitrogen or the normal greenhouse fertilization; and Group B comprised plants that were provided with 25 ppm or 25% of the normal greenhouse fertilization rate. Metabolites were extracted using methanol from root leaf tissue at 10 and 14 weeks after seeding.

The isolated metabolites were analyzed using three different LC/MS approaches (UHPLC-MS/MS (+ESI), UHPLC-MS/MS (-ESI), and GC-MS (+EI)) to separate and identify individual metabolites. Metabolites were identified by comparing the obtained mass spectra to standard spectral databases (Metabolon Inc, Morrisville, NC). Peaks were quantified using area-under-the-curve. Each compound was scaled by registering the medians to equal one (1.00) and normalizing each data point proportionately (termed the “block correction”). The molecular mass of unknown metabolites is provided in Table 1. Discriminant metabolites are shown below in Tables 2 to 5, along with scaled measured values for each sample. Discriminant metabolites were determined by Student's t-test comparisons between TN90 and MD609 taking into account all time points. Metabolites with a p-value less than 0.01 were included in the analysis.

TABLE 1
molecular mass in kiloDaltons for unknown metabolite compounds.

| Metabolite | Mass |
|---|---|
| X-21756 | 247.0918 |
| X-21796 | 138.0566 |
| X-23319 | 299.0771 |
| X-23330 | 251.1136 |
| X-23366 | 189.1023 |
| X-23389 | 157.0762 |
| X-23453 | 161.0818 |
| X-23454 | 319.0933 |
| X-23576 | 267.1237 |
| X-23580 | 311.1136 |
| X-23852 | 374.144 |
| X-23916 | 395.0291 |
| X-23937 | 161.0819 |

TABLE 2
Metabolites negatively correlated with enhanced nitrogen efficiency
identified in root tissue when comparing MD609 and TN90 tobacco lines
at week 10 and week 14 after seeding.

| | TN90 | | | | MD609 | | | |
|---|---|---|---|---|---|---|---|---|
| | 25% | | 100% | | 25% | | 100% | |
| | Nitrogen | | Nitrogen | | Nitrogen | | Nitrogen | |
| Metabolite | W10 | W14 | W10 | W14 | W10 | W14 | W10 | W14 |
| X-23576 | 4.6 | 3.6 | 1.2 | 8.4 | 2.5 | 1.2 | 1 | 2.3 |
| N-acetylmuramate | 0.3 | 0.4 | 2.5 | 4 | 0.1 | 0.2 | 0.3 | 0.6 |
| X-23319 | 0.5 | 0.5 | 3.2 | 2.9 | 0.3 | 0.3 | 0.4 | 1.2 |
| X-23852 | 0.8 | 1.0 | 2.2 | 3.1 | 0.1 | 1.0 | 0.9 | 0.4 |
| X-23330 | 0.7 | 0.5 | 3.5 | 2.4 | 0.4 | 0.6 | 0.9 | 1.0 |
| Alpha-ketoglutarate | 2.1 | 1.5 | 1.5 | 0.9 | 0.8 | 0.6 | 0.8 | 0.5 |
| X-21756 | 0.6 | 0.4 | 1.9 | 1.3 | 0.2 | 0.3 | 1.0 | 0.5 |
| 4-hydroxy-2- | 0.9 | 0.4 | 1.1 | 1.2 | 0.5 | 0.4 | 0.5 | 0.6 |
| oxoglutaric acid | | | | | | | | |
| X-23937 | 0.2 | 0.2 | 0.7 | 1.1 | 0.1 | 0.2 | 0.1 | 0.4 |
| X-23916 | 0.6 | 0.6 | 0.5 | 1.0 | 0.3 | 0.3 | 0.2 | 0.3 |
| 1-methyladenine | 1.2 | 0.9 | 1.2 | 1.1 | 1.0 | 0.8 | 0.8 | 0.7 |

TABLE 3
Metabolites positively correlated with enhanced nitrogen efficiency
identified in root tissue when comparing MD609 and TN90 tobacco lines

| | TN90 | | | | MD609 | | | |
|---|---|---|---|---|---|---|---|---|
| | 25% | | 100% | | 25% | | 100% | |
| | Nitrogen | | Nitrogen | | Nitrogen | | Nitrogen | |
| Metabolite | W10 | W14 | W10 | W14 | W10 | W14 | W10 | W14 |
| 4-guanidinobutanoate | 0.7 | 0.7 | 0.7 | 0.6 | 1.0 | 1.1 | 0.8 | 0.7 |
| Syringaldehyde | 0.5 | 0.6 | 0.4 | 0.3 | 0.9 | 1.0 | 0.6 | 0.4 |
| Thiamin | 0.2 | 0.1 | 0.7 | 0.7 | 0.7 | 1.1 | 0.7 | 1.1 |
| p-hydroxybenzaldehyde | 0.4 | 1.0 | 0.8 | 0.9 | 0.6 | 1.6 | 1.4 | 1.5 |

TABLE 4
Metabolites negatively correlated with enhanced nitrogen efficiency
identified in leaf tissue when comparing MD609 and TN90 tobacco lines

| | TN90 | | | | MD609 | | | |
|---|---|---|---|---|---|---|---|---|
| | 25% | | 100% | | 25% | | 100% | |
| | Nitrogen | | Nitrogen | | Nitrogen | | Nitrogen | |
| Metabolite | W10 | W14 | W10 | W14 | W10 | W14 | W10 | W14 |
| X-23453 | 1.2 | 4.9 | 2.2 | 3.9 | 0.9 | 1.3 | 1.3 | 1.9 |
| X-21756 | 1.6 | 0.8 | 2.3 | 2.5 | 0.8 | 0.3 | 1.0 | 0.8 |
| X-11429 | 1.3 | 0.6 | 2.7 | 2.4 | 0.7 | 0.2 | 1.0 | 1.0 |
| X-21796 | 0.7 | 2.0 | 0.7 | 2.0 | 0.2 | 0.6 | 0.2 | 0.5 |
| N′-methylnicotinamide | 0.7 | 1.0 | 1.9 | 1.1 | 0.9 | 0.1 | 0.1 | 0.2 |
| Cotinine | 0.5 | 1.4 | 0.3 | 1.7 | 0.4 | 0.4 | 0.1 | 0.3 |
| X-23389 | 0.9 | 1.2 | 0.4 | 1.2 | 0.5 | 0.3 | 0.1 | 0.2 |
| N-acetylarginine | 1.0 | 0.7 | 0.8 | 1.9 | 0.6 | 0.3 | 0.7 | 0.8 |
| X-23366 | 0.6 | 0.9 | 0.1 | 0.8 | 0.3 | 0.2 | 0.1 | 0.1 |
| N-acetylphenylalanine | 1.0 | 1.0 | 1.2 | 1.0 | 0.9 | 0.4 | 0.7 | 0.5 |
| Naringenin | 0.4 | 0.8 | 0.3 | 0.8 | 0.2 | 0.2 | 0.1 | 0.4 |

TABLE 5
Metabolites positively correlated with enhanced nitrogen efficiency
identified in leaf tissue when comparing MD609 and TN90 tobacco lines

| | TN90 | | | | MD609 | | | |
|---|---|---|---|---|---|---|---|---|
| | 25% | | 100% | | 25% | | 100% | |
| | Nitrogen | | Nitrogen | | Nitrogen | | Nitrogen | |
| Metabolite | W10 | W14 | W10 | W14 | W10 | W14 | W10 | W14 |
| 4-guanidinobutanoate | 0.6 | 0.8 | 1.0 | 1.1 | 1.3 | 0.9 | 1.6 | 1.6 |
| X-23454 | 0.1 | 0.1 | 0.5 | 0.1 | 0.8 | 0.1 | 1.5 | 1.5 |
| X-23580 | 1.1 | 3.6 | 1.8 | 1.5 | 4.9 | 6.0 | 3.5 | 5.3 |
| X-23852 | 0.9 | 7.7 | 3.6 | 2.1 | 9.8 | 13.2 | 7.6 | 11.0 |

### Example 3. Identification of Gene Expression Associated with Enhanced Nitrogen Utilization Efficiency

The same plants used in Example 1 are also subjected to RNA extraction to be used for RNAseq. RNA is extracted from leaf and root tissue at 10 weeks and 14 weeks after seeding and used for Illumina sequencing. The RNAseq data were analyzed according to methods standard in the art. Candidate genes are subsequently verified.

Seventeen genes (Tables 6 and 7) were found to negatively correlate with the enhanced nitrogen utilization efficiency phenotype of MD609, and seven genes (Tables 8 and 9) were found to positively correlate with the enhanced nitrogen utilization efficiency phenotype of MD609. The negatively correlated genes are candidates for down-regulation in Burley tobacco varieties (via mutagenesis, cisgenic transformation, or transgenic transformation), and the positively correlated genes are candidates for over-expression in Burley tobacco varieties to improve nitrogen utilization efficiency. Single nucleotide polymorphism (SNP) markers associated are provided for tracking the each candidate gene (Tables 6 to 10). The polymorphism associated with the MD609 alleles, and therefore favorable for enhanced NUE is provided (Table 10).

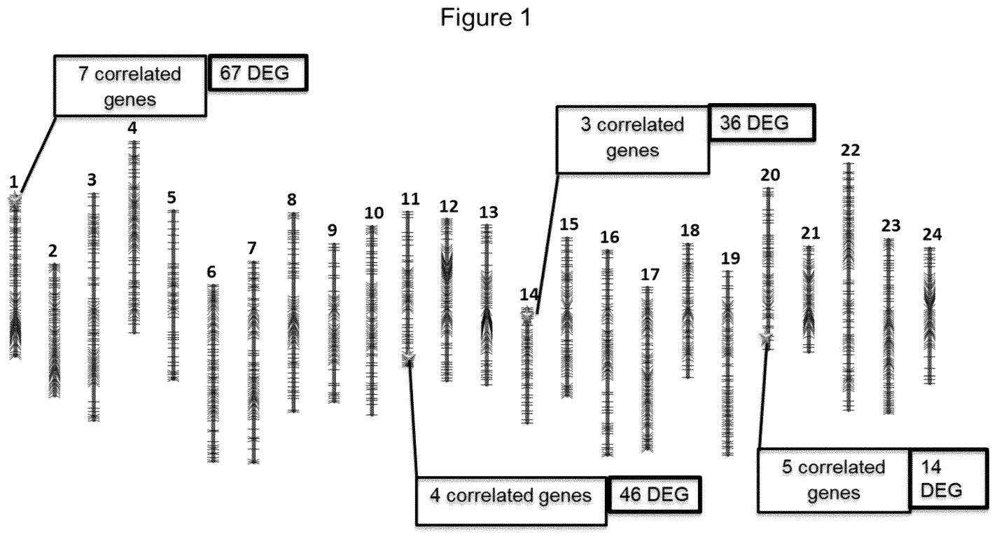

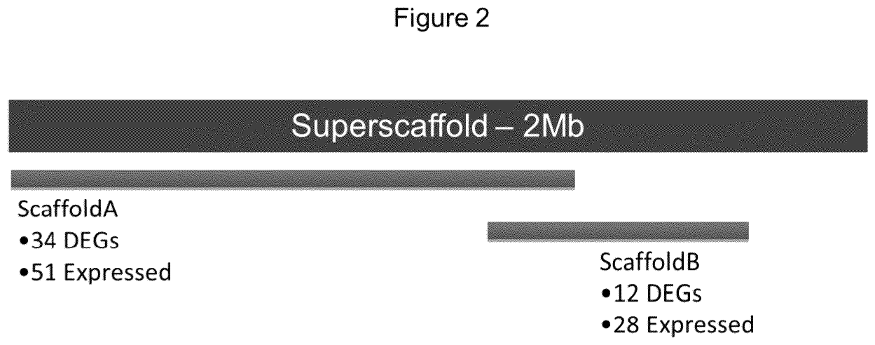

Identification of the genomic location for each of the correlated genes identifies four clusters of genes associated with enhanced NUE in the tobacco genome (FIG. 1). Seven genes are similarly located on chromosome 1, four genes are similarly located on chromosome 11, three genes are similarly located on chromosome 14, and five genes are similarly located on chromosome 20 (FIG. 1). These four locations are also hotspots for genes differentially expressed between low and normal nitrogen conditions (FIG. 1). SNP markers are created to identify MD609 specific and therefore enhanced NUE polymorphisms for each ofthese locations (Tables 6 to 10). The area on chromosome 11 is further characterized and contains 79 total expressed genes and 46 ofthese genes are differentially expressed genes under low nitrogen conditions (FIG. 2).

TABLE 6
Genes identified as negatively correlated with enhanced nitrogen
utilization efficiency in root tissue.

| | | | SNP |
|---|---|---|---|
| | | | marker |
| | SEQ | | SEQ |
| Gene | ID | | ID |
| Identifier | NO | Gene Description | NO: |
| G38453 | 25 | Putative vacuolar proton ATPase subunit E | 57 |
| G64360 | 26 | Clathrin interactor epsin 1-like | 63 |
| G26157 | 27 | Serine/threonine-protein kinase PBS1 | 59 |
| G54692 | 28 | ATPase family AAA domain-containing | 57 |
| | | protein 1-a-like | |
| G32111 | 29 | Uncharacterized protein | 57 |
| G49619 | 30 | Coatomer subunit gamma | 61 |
| G19982 | 31 | Uncharacterized protein | 60 |
| G39737 | 32 | Uncharacterized protein | 58 |
| G28894 | 33 | Putative quinolinate phosphoribosyltransferase | 60 |
| G30288 | 38 | Probable acyl-activating enzyme | 59 |
| | | chloroplastic-like | |
| G39762 | 39 | Alpha-l-fucosidase | 58 |
| G39442 | 40 | Uncharacterized protein | 57 |

TABLE 7
Genes identified as negatively correlated with enhanced nitrogen
utilization efficiency in leaf tissue.

| | | | SNP |
|---|---|---|---|
| | | | Marker |
| | SEQ | | SEQ |
| Gene | ID | | ID |
| Identifier | NO | Gene Description | NO: |
| G41803 | 34 | ABC transporter F-family member 3-like | 57 |
| G46356 | 35 | Uncharacterized protein | 57 |
| G56420 | 36 | WD repeat-containing protein 26-like | 58 |
| G59801 | 37 | Protein phosphatase 2A | 60 |
| G30288 | 38 | Probable acyl-activating enzyme | 59 |
| | | chloroplastic-like | |
| G39762 | 39 | Alpha-l-fucosidase | 58 |
| G39442 | 40 | Uncharacterized protein | 57 |

TABLE 8
Genes identified as positively correlated with enhanced nitrogen
utilization efficiency in root tissue.

| | | | SNP |
|---|---|---|---|
| | | | Marker |
| | SEQ | | SEQ |
| Gene | ID | | ID |
| Identifier | NO | Gene Description | NO: |
| G59318 | 1 | PR-10 type pathogenesis-related protein | 57 |
| G20580 | 2 | Uncharacterized amino acid permease | 60 |
| G30999 | 3 | TBZ17 | 62 |
| G29260 | 4 | BTB/POZ domain-containing protein | 64 |
| | | (AT5G48800-like) | |
| G41446 | 8 | 3-isopropylmalate dehydratase small | 57 |
| | | subunit | |

TABLE 9
Genes identified as positively correlated with enhanced nitrogen
utilization efficiency in leaf tissue.

| | | | SNP |
|---|---|---|---|
| | | | Marker |
| | SEQ | | SEQ |
| Gene | ID | | ID |
| Identifier | NO | Gene Description | NO: |
| G41343 | 5 | Glucose-6-phosphate 1-epimerase-like | 57 |
| G53261 | 6 | Probable nitrite transporter | 60 |
| | | (AT1G68570-like) | |
| G42290 | 7 | Phospho-2-dehydro-3-deoxyheptonate | 58 |
| | | aldolase | |
| G41446 | 8 | 3-isopropylmalate dehydratase small | 61 |
| | | subunit | |

TABLE 10
SNP markers comprising polymorphisms associated with
enhanced NUE.

| SNP marker | Positon of | Allele associated |
|---|---|---|
| SEQ ID NO | polymorphism | with NUE |
| 57 | 147 | T |
| 58 | 57 | G |
| | 117 | C |
| 59 | 162 | G |
| 60 | 36 | C |
| 61 | 36 | T |
| 62 | 36 | T |
| 63 | 36 | G |
| 64 | 36 | T |

### Example 4. Identifying Tobacco Leaf- and Root-Preferred Promoters

RNA samples from 4 week old TN90 tobacco plants are obtained from 10 tissue types (axillary buds before topping; axillary buds 2 hours after topping; axillary buds 6 hours after topping; axillary buds 24 hours after topping; axillary buds 72 hours after topping; roots before topping; roots 24 hours after topping; roots 72 hours after topping; young leaf at the time of topping; and shoot apical meristem). The resulting RNA samples (three independently collected samples for each tissue type) are used as starting material for Illumina 1×100 bp sequencing.

Illumina reads are mapped and used to identify a list of candidate genes exhibiting high root or leaf expression. Tables 11 and 12 provide RPKM expression values for genes identified as having leaf-preferred or root-preferred expression. These genes are candidates for possessing leaf-preferred promoters or root-preferred promoters, respectively.

TABLE 11
Genes with leaf-preferred expression

| | | SEQ ID | Axillary Bud | | | | | Root | | | | |
|---|---|---|---|---|---|---|---|---|---|---|---|---|
| Promoter | Gene Description | NO: | 0 hr | 2 hr | 6 hr | 24 hr | 72 hr | 0 hr | 24 hr | 72 hr | SAM | Leaf |
| P16098 | Carbonic anhydrase | 17 | 4.88 | 5.94 | 7.49 | 4.67 | 16.12 | 0.45 | 0.41 | 0.52 | 2.89 | 1002.14 |
| P42207 | CP12 | 18 | 0.41 | 0.99 | 1.24 | 0.52 | 1.83 | 0.05 | 0.02 | 0.07 | 0.13 | 34.34 |
| P47582 | Chloroplast | 19 | 0.32 | 0.68 | 0.91 | 0.68 | 1.96 | 0.03 | 0.03 | 0.06 | 0.06 | 96.69 |
| | sedoheptulose-1,7- | | | | | | | | | | | |
| | bisphosphatase | | | | | | | | | | | |

TABLE 12
Genes with root-preferred expression

| | | SEQ ID | Axillary Bud | | | | | Root | | | | |
|---|---|---|---|---|---|---|---|---|---|---|---|---|
| Promoter | Gene Description | NO: | 0 hr | 2 hr | 6 hr | 24 hr | 72 hr | 0 hr | 24 hr | 72 hr | SAM | Leaf |
| P2862 | Putative PLA2 | 20 | 0.65 | 0.78 | 0.58 | 0.38 | 0.38 | 336.69 | 391.95 | 511.86 | 0.36 | 0.43 |
| P57190 | Uncharacterized | 21 | 0.38 | 0.45 | 0.29 | 0.39 | 0.35 | 198.00 | 416.84 | 384.52 | 0.47 | 0.26 |
| | protein | | | | | | | | | | | |
| P49330 | Glutathione S- | 22 | 0.35 | 0.35 | 0.27 | 0.75 | 0.38 | 196.29 | 269.39 | 417.71 | 0.23 | 0.22 |
| | transferase parC | | | | | | | | | | | |
| P3788 | PR-10 type | 23 | 0.29 | 0.36 | 0.45 | 0.15 | 0.23 | 192.16 | 88.51 | 193.35 | 0.26 | 0.16 |
| | pathogenesis- | | | | | | | | | | | |
| | related | | | | | | | | | | | |
| | protein | | | | | | | | | | | |
| P77628 | Cytochrome P450 | 24 | 0.39 | 0.71 | 0.53 | 0.39 | 0.44 | 144.99 | 333.54 | 386.32 | 0.52 | 0.50 |

### Example 5. Development of Modified Plants

An expression vector, p45-2-7 (SEQ ID NO: 65), is used as a backbone to generate multiple transformation vectors (See Examples X-Y). p45-2-7 contains a CsVMV promoter, a NOS terminator, and a cassette comprising a kanamycin selection marker (NPT II) operably linked to an Actin2 promoter and a NOS terminator. Nucleic acid vectors comprising transgenes of interest are introduced into tobacco leaf discs via Agrobacterium transformation. See, for example, Mayo et al., 2006, Nat Protoc. 1:1105-11 and Horsch et al., 1985, Science 227:1229-1231.

TN90 tobacco plants are grown in Magenta™ GA-7 boxes and leaf discs are cut and placed into Petri plates. Agrobacterium tumefaciens cells comprising a transformation vector are collected by centrifuging a 20 mL cell suspension in a 50 mL centrifuge tube at 3500 RPM for 10 minutes. The supernatant is removed and the Agrobacterium tumefaciens cell pellet is resuspended in 40 mL liquid re-suspension medium. Tobacco leaves, avoiding the midrib, are cut into eight 0.6 cm discs with a #15 razor blade and placed upside down in a Petri plate. A thin layer of Murashige & Skoog with B5 vitamins liquid re-suspension medium is added to the Petri plate and the leaf discs are poked uniformly with a fine point needle. Approximately 25 mL of the Agrobacterium tumefaciens suspension is added to the Petri plate and the leaf discs are incubated in the suspension for 10 minutes.

Leaf discs are transferred to co-cultivation Petri plates (1/2 MS medium) and discs are placed upside down in contact with filter paper overlaid on the co-cultivation TOM medium (MS medium with 20 g/L sucrose; 1 mg/L indole-3-acetic acid; and 2.5 mg/L 6-benzyl aminopurine (BAP)). The Petri plate is sealed with parafilm prior to incubation in dim light (60-80 mE/ms) with 18 hours on, 6 hours off photoperiods at 24 degrees Celsius for three days. After incubation, leaf discs are transferred to regeneration/selection TOM K medium Petri plates (TOM medium plus 300 mg/L kanamycin). Leaf discs are sub-cultured bi-weekly to fresh TOM K medium in dim light with 18 hours on, 6 hours off photoperiods at 24 degrees Celsius until shoots become excisable. Shoots from leaves are removed with forceps and inserted in MS basal medium with 100 mg/L kanamycin. Shoots on MS basal medium with 100 mg/L kanamycin are incubated at 24 degrees Celsius with 18 hours on, 6 hours off photoperiods with high intensity lighting (6080 mE/ms) to induce rooting.

When plantlets containing both shoots and roots grow large enough (e.g., reach approximately half the height of a Magenta™ GA-7 box), they are transferred to soil. Established seedlings are transferred to a greenhouse for further analysis and to set seed. Evaluation of enhanced nitrogen utilization efficiency phenotypes is conducted by growing modified plants (T₀, T₁, T₂, or later generations) and control plants. Control plants are either NLM plants that have not been transformed or NLM plants that have been transformed with an empty p45-2-7 vector.

Phenotypic screening for enhanced nitrogen utilization efficiency is conducted in a greenhouse using zero parts per million (ppm) nitrogen (no nitrogen), 25 ppm nitrogen (low nitrogen), and 100 ppm nitrogen (normal nitrogen). Initial screening is undertaken in the greenhouse with T₁ plants. Homozygous T₂ populations are then evaluated in the field using 60 pounds per acre fertilizer (˜25% of the recommended rate for Burley tobacco. Seedling growth, chlorophyll loss, and final yield are measured and compared to control plants grown at normal nitrogen levels.

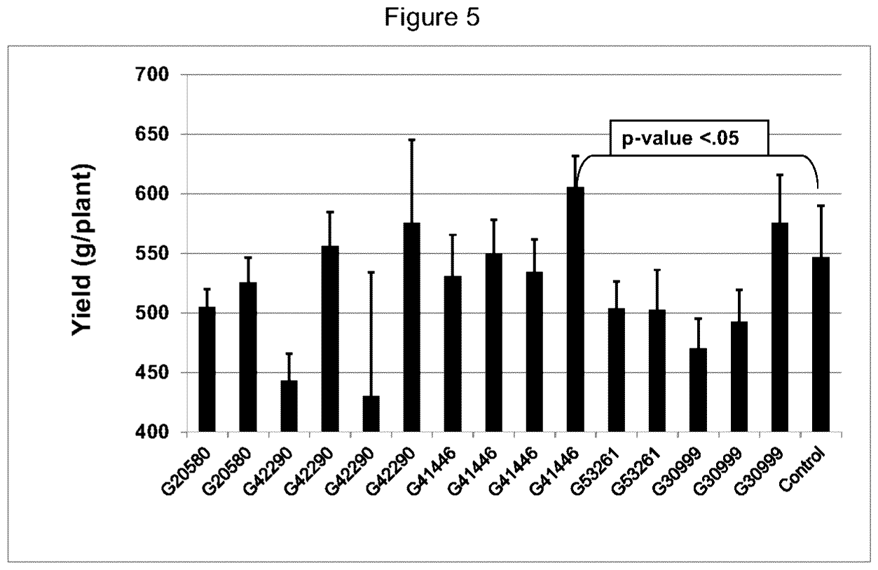

In the T₁ generation, plants overexpressing G20580 (2 independent transformants), G42290 (4 independent transformants), G41446 (4 independent transformants), G53261 (2 independent transformants), and G30999 (3 independent transformants) are grown in the greenhouse along with controls under nitrogen limiting conditions equivalent to 60 pounds of Nitrogen per acre. Nine plants per transformant are sampled and one of the lines overexpressing G41446 show a statistically significant increase in yield (grams fresh weight per plant) compared to the control (See FIG. 5).

### Example 6. Creating a Cisgenic Tobacco Plant with Enhanced Nitrogen Utilization Efficiency

Nitrogen utilization efficiency can be improved by modifying the expression of genes involving the genes that were identified as differentially expressed in Example 2. Similarly, genes involved in the biosynthesis or degradation of the metabolites identified in Example 1 can be modulated to improve nitrogen utilization efficiency. Genes that are positively associated with enhanced nitrogen utilization efficiency can be over-expressed using a general over-expression promoter or a tissue-preferred promoter to over-express the gene in desired tissues.

Transformation vectors are created to overexpress proteins that are positively associated with enhanced nitrogen utilization efficiency. Separate transformation vectors comprising one of SEQ ID NOs:9 to 16 are incorporated into p45-2-7 transformation vectors. Additionally transformation vectors are created comprising one of SEQ ID NOs:9 to 16.

Modified tobacco plants are generated using these transformation vectors according to Example 4. Modified tobacco plants (T₁ generation) and control tobacco plants are then phenotypically evaluated as described in Example 4. The modified tobacco plants exhibit enhanced nitrogen utilization efficiency as compared to control tobacco plants grown under the same conditions.

### Example 7. Creating a Transgenic Tobacco Plant with Enhanced Nitrogen Utilization Efficiency

Nitrogen utilization efficiency can also be enhanced by down-regulating the expression of genes identified as being negatively associated with nitrogen utilization efficiency in Example 2.

Transformation vectors comprising RNAi constructs are designed to inhibit tobacco genes whose expression is negatively associated with nitrogen utilization efficiency in Example 2. Separate transformation vectors comprise one of SEQ ID NOs:41 to 56, which are incorporated into p45-2-7 transformation vectors. Additional transformation vectors are created comprising one of SEQ ID NOs:41 to 56.

Modified tobacco plants are generated using these transformation vectors according to Example 4. Modified tobacco plants (T1 generation) and control tobacco plants are then phenotypically evaluated as described in Example 4. The modified tobacco plants exhibit enhanced nitrogen utilization efficiency as compared to control tobacco plants grown under the same conditions.

### Example 8. Additional Methods of Improving Nitrogen Utilization Efficiency Using Gene Editing Technologies

Gene editing technologies such as CRISPR/Cas9, CRISPR/Cpf1, CRISPR/CasX, CRISPR/CasY, CRISPR/Csm1, zinc-finger nucleases (ZFN), and transcription activator-like effector nucleases (TALENs) are used to modify the coding region of a gene negatively associated with enhanced nitrogen utilization efficiency so that the gene encodes a non-functional protein or a lower-functioning protein. These gene editing technologies are also used to edit or replace an endogenous promoter sequence to drive its cognate protein expression in either leaf or root tissue to improve nitrogen utilization efficiency. For example, an endogenous G64360 is edited or replaced so the gene is only expressed in leaf tissue, where it can function to improve nitrogen utilization efficiency of the plant.

Separate CRISPR/Cas9 or CRISPR/Cpf1 guide RNAs are constructed to recognize and hybridize to the promoter sequence of each one of SEQ ID NOs:9 to 40. The engineered guide RNA and a donor polynucleotide comprising a promoter selected from the group consisting of SEQ ID NOs: 17 to 24 are provided to a tobacco plant, allowing the selected promoter to replace the endogenous promoter of the selected genes and restrict expression of endogenous to either leaf or root tissue as desired. The edited tobacco plants exhibit enhanced nitrogen utilization efficiency compared to control tobacco plants grown under similar conditions.

### Example 9. Development of Novel Mutations to Improve Nitrogen Utilization Efficiency Via Random Mutagenesis

Random mutagenesis of tobacco plants are performed using ethyl methanesulfonate (EMS) mutagenesis or fast neutron bombardment. EMS mutagenesis consists of chemically inducing random point mutations. Fast neutron mutagenesis consists of exposing seeds to neutron bombardment which causes large deletions through double stranded DNA breakage.

For EMS mutagenesis, one gram (approximately 10,000 seeds) of the Burley tobacco variety TN90 seeds are washed in 0.1% Tween for fifteen minutes and then soaked in 30 mL of ddH₂O for two hours. One hundred fifty (150) μL of 0.5% EMS (Sigma, Catalogue No. M-0880) is then mixed into the seed/ddH₂O solution and incubated for 8-12 hours (rotating at 30 R.P.M.) under a hood at room temperature (RT; approximately 20° C.).The liquid then is removed from the seeds and mixed into 1 M NaOH overnight for decontamination and disposal. The seeds are then washed twice with 100 mL ddH₂O for 2-4 hours. The washed seeds are then suspended in 0.1% agar solution.

The EMS-treated seeds in the agar solution are evenly spread onto water-soaked Carolina's Choice Tobacco Mix (Carolina Soil Company, Kinston, NC) in flats at ˜2000 seeds/flat. The flats are then covered with plastic wrap and placed in a growth chamber. Once the seedlings emerge from the soil, the plastic wrap is punctured to allow humidity to decline gradually. The plastic wrap is completely removed after two weeks. Flats are moved to a greenhouse and fertilized with NPK fertilizer. The seedlings are replugged into a float tray and grown until transplanting size. The plants are subsequently transplanted into a field. During growth, the plants self-pollinate to form M1 seeds. At the mature stage, five capsules are harvested from each plant and individual designations are given to the set of seeds from each plant. This forms the M1 population. A composite of M1 seed from each M0 plant are grown, and plants are phenotypically evaluated for enhanced nitrogen efficiency as described in Example 4. M1 plants exhibiting enhanced nitrogen efficiency are selected and screened for mutations using DNA sequencing and gene mapping techniques known in the art.

### Example 10. Using Breeding to Create a Tobacco Plant with Enhanced Nitrogen Utilization Efficiency

Traditional breeding techniques can be used to introduce NUE favorable alleles provided herein into any tobacco variety to enhance NUE. A population of tobacco plants can be created by crossing a tobacco plant with at least one favorable NUE allele (See Table 10) to a tobacco plant lacking that favorable allele. Marker assisted selection, or other techniques known in the art (e.g. direct sequencing) can be used to track introgression of a favorable allele in the F₁ generation and can be used to determine heterozygosity or homozygosity in subsequent generations. Enhanced NUE of progeny plants can be determined using methods known in the art or described above. Multiple different NUE favorable alleles can be combined into a single line. A molecular phenotype as determined by metabolite signature can be used to track enhanced NUE during breeding. The metabolite signatures of progeny plants can be determined using methods described above. Progeny plants with metabolite signatures of parental plants with enhanced NUE are crossed to create subsequent populations of tobacco plants with enhanced NUE.

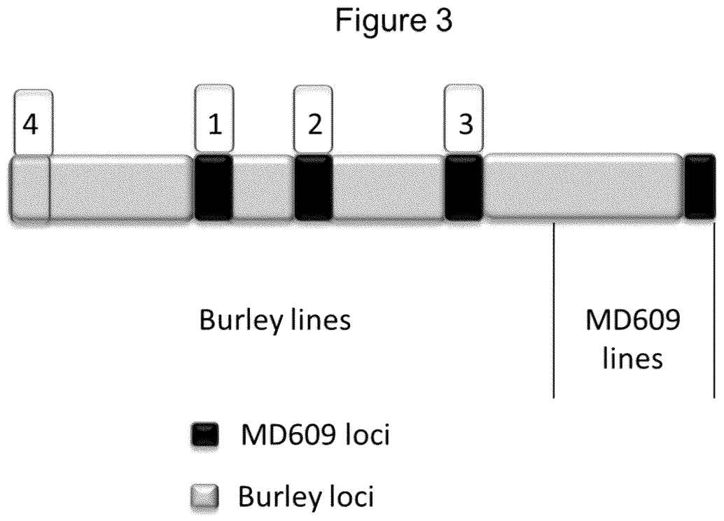

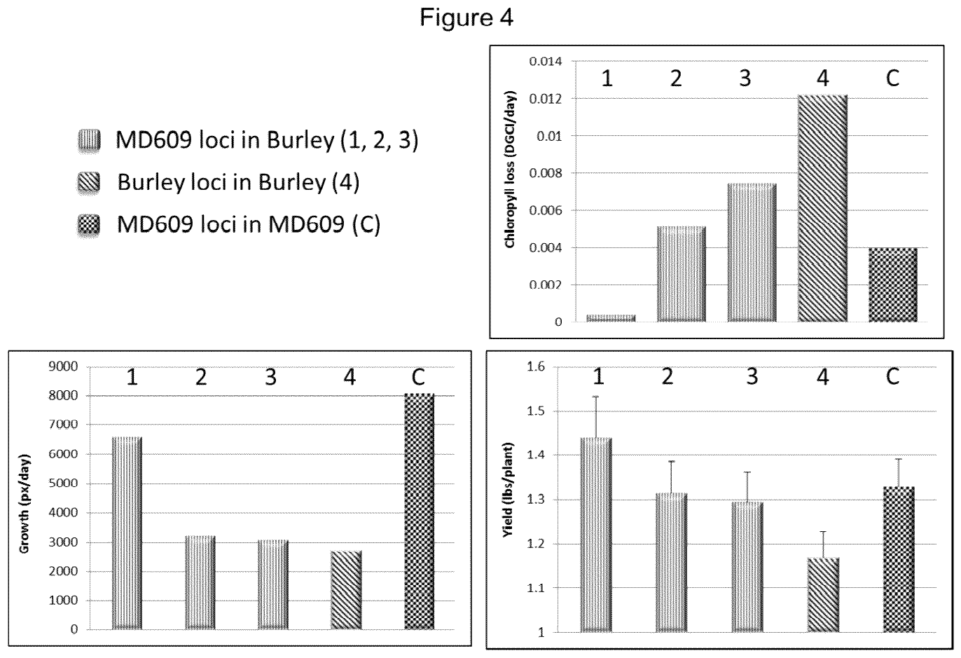

Introduction of Maryland609 loci into commercially available Burley varieties can be performed as described to develop Burley lines with enhanced NUE. Screening of 23 Burley and 6 MD609 lines identified 3 Burley lines containing the MD609 allele at SNP marker S451 (SEQ ID NO:58) (FIG. 3). The three Burley lines with the MD609 allele were tested for chlorophyll loss, growth, and yield under nitrogen limiting conditions and compared to a control TN90 Burley line and a control MD609 line (MD609 with the MD609 allele at SNP marker S451) (FIG. 4). The Burley lines with the MD609 allele exhibit chlorophyll lose, growth, and yield more similar to the Maryland control (FIG. 4). The TN90 Burley control exhibits increased chlorophyll lose, decreased growth, and decreased yield compared to the MD609 control (FIG. 4). These results indicate that introduction of the MD609 allele at SNP marker S451 can enhance NUE.

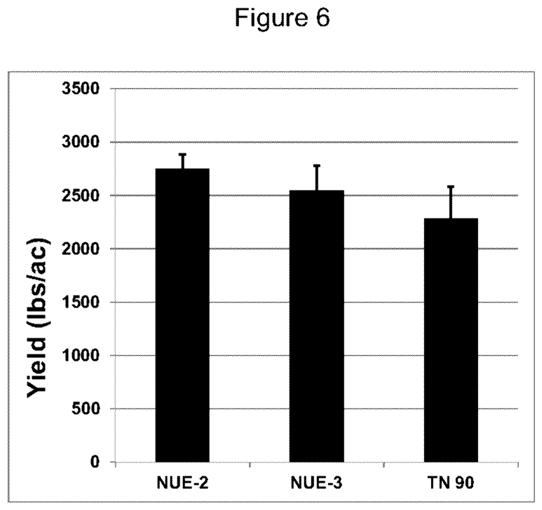

In order to introduce MD609 alleles into Burley, MD609 was crossed with Burley. F₁ progeny from this cross were selected and subsequently selfed to produce F₂ seed. F₂ and F₃ plants were grown and selfed to generate F₄ seed. Bulked F₄ seed from two independent crossing schemes, identified as the NUE-2 and NUE-3 lines, are grown and harvested in the field. The genotypes of the SNP markers S451, S317, S12385, S238, S3894, and S2237 are determined for F₄ seed of both NUE-2 and NUE-3 lines (See Table 13). F₄ plants are grown using reduced nitrogen production practices described in Example 1. Both NUE-2 and NUE-3 lines demonstrate an increased yield in pounds per acre compared to the Burley control TN90 (See FIG. 6).

Alternatively, a modified tobacco plant comprising an enhanced NUE phenotype can be created using the methods described herein and crossed to a unmodified tobacco plant to propagate the modification in subsequent generations. Selection for the genetic modification can be tracked using appropriate techniques known in the art. Enhanced NUE of progeny plants can be determined using methods known in the art or described above.

TABLE 13
Genotypes of field grown plants from F₄ NUE-2 and NUE-3 lines and TN90.
MD represents a MD609 allele, Burley represents a Burley allele,
and HET represents a heterozygous MD609/Burley.

| | S451 | S317 | S12835 | S238 | S3894 | S2237 |
|---|---|---|---|---|---|---|
| NUE-2 | MD | HET | MD | Burley | Burley | MD |
| NUE-3 | MD | HET | MD | Burley | Burley | MD |
| TN90 | Burley | Burley | Burley | Burley | Burley | Burley |

1-20. (canceled)

21. A method of producing a tobacco plant comprising an enhanced nitrogen utilization efficiency (NUE) trait, said method comprising:
a. providing a first population of tobacco plants comprising at least one tobacco plant comprising an enhanced NUE trait;
b. genotyping said first population of tobacco plants via a molecular assay for the presence of one or more molecular markers located within 20 cM of a SNP marker selection from the group consisting of SEQ ID NOs: 59, 60, 61, 62, 63, and 64; and
c. selecting a tobacco plant comprising said one or more molecular markers.

22. The method of claim 21, further comprising:
d. crossing said tobacco plant selected in step (c) with a second tobacco plant; and
e. obtaining progeny seed from the cross of step (d) wherein a plant grown from said progeny seed comprises an enhanced NUE trait and said SNP marker.

23. The method of claim 21, wherein said SNP marker comprises a favorable NUE allele selected from the group consisting of:
a G nucleotide at a position corresponding to position 162 of SEQ ID NO: 59,
a C nucleotide at a position corresponding to position 36 of SEQ ID NO: 60,
a T nucleotide at a position corresponding to position 36 of SEQ ID NO: 61,
a T nucleotide at a position corresponding to position 36 of SEQ ID NO: 62,
a G nucleotide at a position corresponding to position 36 of SEQ ID NO: 63, and
a T nucleotide at a position corresponding to position 36 of SEQ ID NO: 64.

24. The method of claim 21, wherein said enhanced NUE trait is selected from the group consisting of an increased partial factor productivity (PFP), an increased agronomic efficiency (AE), an increased recovery efficiency (RE), an increased physiological efficiency (PE), and an increased internal efficiency (IE), when compared to a tobacco plant lacking said enhanced NUE trait when grown in the same conditions.

25. The method of claim 21, wherein said first population of tobacco plants is selected from the group consisting of MD609, MD601, Banket A1, K326, K346, K358, K394, K399, K730, NC196, NC37NF, NC471, NC55, NC92, NC2326, NC95, and NC925.

26. The method of claim 21, wherein said second tobacco plant is a Burley tobacco variety.

27. The method of claim 21, wherein said second tobacco plant is selected from the group consisting of TN86, TN86LC, TN90, TN90LC, TN97, and TN97LC.

28. The method of claim 21, wherein said one or more molecular markers are within 10 cM of a favorable NUE allele selected from the group consisting of:
a G nucleotide at a position corresponding to position 162 of SEQ ID NO: 59,
a C nucleotide at a position corresponding to position 36 of SEQ ID NO: 60,
a T nucleotide at a position corresponding to position 36 of SEQ ID NO: 61,
a T nucleotide at a position corresponding to position 36 of SEQ ID NO: 62,
a G nucleotide at a position corresponding to position 36 of SEQ ID NO: 63, and
a T nucleotide at a position corresponding to position 36 of SEQ ID NO: 64.

29. The method of claim 21, wherein said one or more molecular markers are within 5 cM of a favorable NUE allele selected from the group consisting of:
a G nucleotide at a position corresponding to position 162 of SEQ ID NO: 59,
a C nucleotide at a position corresponding to position 36 of SEQ ID NO: 60,
a T nucleotide at a position corresponding to position 36 of SEQ ID NO: 61,
a T nucleotide at a position corresponding to position 36 of SEQ ID NO: 62,
a G nucleotide at a position corresponding to position 36 of SEQ ID NO: 63, and
a T nucleotide at a position corresponding to position 36 of SEQ ID NO: 64.

30. The method of claim 21, wherein said one or more molecular markers are within 1 cM of a favorable NUE allele selected from the group consisting of:
a G nucleotide at a position corresponding to position 162 of SEQ ID NO: 59,
a C nucleotide at a position corresponding to position 36 of SEQ ID NO: 60,
a T nucleotide at a position corresponding to position 36 of SEQ ID NO: 61,
a T nucleotide at a position corresponding to position 36 of SEQ ID NO: 62,
a G nucleotide at a position corresponding to position 36 of SEQ ID NO: 63, and
a T nucleotide at a position corresponding to position 36 of SEQ ID NO: 64.

31. The method of claim 21, wherein said molecular marker is selected from the group consisting of a SNP marker, an INDEL marker, an RFLP marker, an SSR marker, an AFLP marker, and a RAPD marker.

32. A method of introgressing an enhanced NUE trait into a tobacco variety, said method comprising the steps of:
a. crossing a first tobacco variety comprising an enhanced nitrogen utilization efficiency trait with a second tobacco variety lacking said enhanced nitrogen utilization efficiency trait;
b. obtaining progeny seed from the cross of step (a);
c. genotyping at least one of said progeny seed obtained in step (b) via a molecular assay for a molecular marker linked within 20 cM to a SNP marker selection from the group consisting of SEQ ID NOs: 59, 60, 61, 62, 63, and 64; and
d. selecting a progeny seed comprising at least one favorable NUE allele at said SNP marker and further comprising said enhanced nitrogen utilization efficiency trait.

33. The method of claim 32, wherein said SNP marker comprises a favorable NUE allele selected from the group consisting of
a G nucleotide at a position corresponding to position 162 of SEQ ID NO: 59,
a C nucleotide at a position corresponding to position 36 of SEQ ID NO: 60,
a T nucleotide at a position corresponding to position 36 of SEQ ID NO: 61,
a T nucleotide at a position corresponding to position 36 of SEQ ID NO: 62,
a G nucleotide at a position corresponding to position 36 of SEQ ID NO: 63, and
a T nucleotide at a position corresponding to position 36 of SEQ ID NO: 64.

34. The method of claim 32, wherein said second tobacco variety is a Burley tobacco variety.

35. The method of claim 32, wherein said second tobacco variety is selected from the group consisting of TN86, TN86LC, TN90, TN90LC, TN97, and TN97LC.

36. The method of claim 32, where said molecular marker is linked within 10 cM to a SNP marker comprising a favorable NUE allele selected from the group consisting of
a G nucleotide at a position corresponding to position 162 of SEQ ID NO: 59,
a C nucleotide at a position corresponding to position 36 of SEQ ID NO: 60,
a T nucleotide at a position corresponding to position 36 of SEQ ID NO: 61,
a T nucleotide at a position corresponding to position 36 of SEQ ID NO: 62,
a G nucleotide at a position corresponding to position 36 of SEQ ID NO: 63, and
a T nucleotide at a position corresponding to position 36 of SEQ ID NO: 64.

37. The method of claim 32, where said molecular marker is linked within 5 cM to a SNP marker comprising a favorable NUE allele selected from the group consisting of
a G nucleotide at a position corresponding to position 162 of SEQ ID NO: 59,
a C nucleotide at a position corresponding to position 36 of SEQ ID NO: 60,
a T nucleotide at a position corresponding to position 36 of SEQ ID NO: 61,
a T nucleotide at a position corresponding to position 36 of SEQ ID NO: 62,
a G nucleotide at a position corresponding to position 36 of SEQ ID NO: 63, and
a T nucleotide at a position corresponding to position 36 of SEQ ID NO: 64.

38. The method of claim 32, where said molecular marker is linked within 1 cM to a SNP marker comprising a favorable NUE allele selected from the group consisting of
a G nucleotide at a position corresponding to position 162 of SEQ ID NO: 59,
a C nucleotide at a position corresponding to position 36 of SEQ ID NO: 60,
a T nucleotide at a position corresponding to position 36 of SEQ ID NO: 61,
a T nucleotide at a position corresponding to position 36 of SEQ ID NO: 62,
a G nucleotide at a position corresponding to position 36 of SEQ ID NO: 63, and
a T nucleotide at a position corresponding to position 36 of SEQ ID NO: 64.

39. The method of claim 32, wherein said enhanced NUE trait is selected from the group consisting of an increased partial factor productivity (PFP), an increased agronomic efficiency (AE), an increased recovery efficiency (RE), an increased physiological efficiency (PE), and an increased internal efficiency (IE), when compared to a tobacco plant lacking said enhanced NUE trait when grown in the same conditions.

40. The method of claim 32, wherein said first population of tobacco plants is selected from the group consisting of MD609, MD601, Banket A1, K326, K346, K358, K394, K399, K730, NC196, NC37NF, NC471, NC55, NC92, NC2326, NC95, and NC925.
